(19) United States
(12) Patent Application Publication
(10) Pub. No.: US 2003/0215913 A1
(43) Pub. Date: Nov. 20, 2003

(54) Nucleic acids, vectors, host cells, polypeptides, and uses thereof

(76) Inventors: Enrique Alvarez, Clinton, CT (US); Jacquelyn Kaye McEntire, Indianapolis, IN (US); Rosamund Carol Smith, Greenfield, IN (US); Eric Wen Su, Carmel, IN (US)

Correspondence Address: ELI LILLY AND COMPANY, PATENT DIVISION, P.O. BOX 6288, INDIANAPOLIS, IN, 46206-6288, US

(21) Appl. No.: 10/311,784

(22) PCT Filed: Jul. 5, 2001
(86) PCT No.: PCT/US01/16517

(51) Int. Cl. A61K 38/17; C07H 21/04; C07K 14/47; C12P 21/02; C12N 5/06

(52) U.S. Cl. 435/69.1; 435/320.1; 435/325; 530/350; 514/12; 536/23.5

(57) ABSTRACT

The present invention provides nucleic acid sequences encoding novel human proteins. These novel nucleic acids are useful for constructing the claimed DNA vectors and host cells of the invention and for preparing the claimed recombinant proteins and antibodies that are useful in the claimed methods and medical uses.

This application claims priority of Provisional Application Serial No. 60/219,359 filed Jul. 19, 2000, and Serial No. 60/288,892 filed May 4, 2001.

### FIELD OF THE INVENTION

The present invention relates to the identification and isolation of novel DNA, therapeutic and drug discovery uses, and the recombinant production of novel secreted polypeptides, designated herein as LP226, LP233, and LP236, having sequence similarity to human chordin. The present invention also relates to vectors, host cells, and antibodies directed to these polypeptides.

### BACKGROUND OF THE INVENTION

The transforming growth factor beta (TGF-β) family is a protein superfamily of growth factors. These proteins are related by a common motif of conserved cysteines in specific locations. Members of the TGF-beta family are involved in numerous processes. These include regulation of cellular differentiation, wound healing, and bone formation. Additionally, TGF-betas are associated with conditions of disease states such as inflammatory and fibrotic conditions, carcinogenesis, immunosuppressive disorders, neurodegenerative diseases, and coronary artery diseases. Flanders and Roberts, “TGFβ” in Cytokine Reference: A Compendium of Cytokines and Other Mediators of Host Defense 1: 719-38 (J. Oppenheim, ed., 2001).

The TGF-beta superfamily consists of numerous proteins, including a family of growth differentiation factors (GDFs) and a family of bone morphogenetic proteins (BMPs). BMPs regulate diverse biological processes. Particularly, individual vertebrate BMPs are involved in embryogenesis and may be key regulators of organ development. Wozney and Rosen, Clin. Orthop. Rel. Res. 346: 26-37 (1998). For example, BMP-2 and BMP-4 have been shown to be fundamental regulators of skeletal tissue formation and repair. Wozney and Rosen. BMPs and their receptors may be required to promote bone regeneration following fracture. Schmitt, et al., J. Orthop. Res. 17(2): 269-78 (1999). Thus, regulation of cell growth and differentiation activities via regulation of BMPs may be therapeutically useful for tissue and bone regeneration as well as wound healing.

However, BMPs are potent mitogens and morphogens that may also be involved in cell differentiation associated with tumor growth. BMPs, as well as other TGF-beta superfamily proteins, are abundant in bone, and may be an important factor in the establishment and progression of cancer metastases to bone. Guise and Mundy, Endocrine Reviews 19(1): 18-54 (1998). In instances where cell growth is excessive or otherwise deleterious, such as in carcinogenesis, inhibition of BMPs and other TGF-beta family members may be warranted to decrease or stop cell growth. This inhibition may be accomplished using naturally occurring inhibitors, antibodies, or small molecules.

Chordin and chordin related proteins are naturally occurring inhibitors of some BMPs and other TGF-beta family members. Chordin-type proteins contain a cysteine-rich region, typically identified by the following formula: CX₂₄CX₂CX₁CX₆₋₈CX₄CX₄CX₁₂CCX₂CX₃R/K. In human chordin, this cysteine-rich motif repeats four times throughout the sequence. This region interacts antagonistically to directly bind some TGF-beta family members, yielding regulation of cartilage and skeletal morphogenesis. Schmitt, supra. Additionally, chordin and similar proteins may be involved in organogenesis and homeostasis. Chordin is expressed in both fetal and adult tissues, particularly in the liver and cerebellum. Pappano, et al., Genomics 52(2): 236-9 (1998).

LP226, LP233, and LP236 are proteins which have significant sequence similarity to the cysteine-rich regions of chordin. DNA encoding LP226, LP233, and LP236 is found in the human chromosomal region 11q14, which has been genetically mapped to disease states including schizophrenia, tuberous sclerosis, prepubertal periodontitis, Papillon-Lefevre syndrome, salivary gland mucoepidermoid carcinoma, chronic lymphocytic leukemia, and T cell lymphoblastic leukemia.

More generally, all novel proteins are of interest. Extracellular proteins play an important role in the formation, differentiation and maintenance of multicellular organisms. The fate of many individual cells, e.g., proliferation, migration, differentiation, or interaction with other cells, is typically governed by information received from other cells and/or the immediate environment. This information is often transmitted by secreted polypeptides (for instance, mitogenic factors, survival factors, cytotoxic factors, differentiation factors, neuropeptides, and hormones) which are, in turn, received and interpreted by diverse cell receptors or membrane-bound proteins. These secreted polypeptides or signaling molecules normally pass through the cellular secretory pathway to reach their site of action in the extracellular environment.

Secreted proteins have various industrial applications, including pharmaceuticals, diagnostics, biosensors and bioreactors. Most protein drugs available at present, such as thrombolytic agents, interferons, interleukins, erythropoietins, colony stimulating factors, and various other cytokines, are secretory proteins. Their receptors, which are membrane proteins, also have potential as therapeutic or diagnostic agents.

The present invention describes the cloning and characterization of novel proteins, termed LP226, LP233, and LP236 and active variants, and fragments thereof.

### SUMMARY OF THE INVENTION

The present invention provides isolated LP226, LP233, and LP236 encoding nucleic acids and polypeptides, including fragments and variants thereof. Contemplated by the present invention are LP226, LP233, and LP236 probes, primers, recombinant vectors, host cells, transgenic animals, chimeric antibodies and constructs, LP226, LP233, and LP236 epitope recognizing antibodies, as well as methods of making and using them diagnostically and therapeutically as described and enabled herein.

The present invention includes isolated nucleic acid molecules comprising a polynucleotide that encodes an LP226, LP233, or LP236 polypeptide as defined herein, as well as variants thereof, or an isolated nucleic acid molecule that is complementary to a polynucleotide that encodes an LP226, LP233, or LP236 polypeptide or a variant thereof as defined herein.

A polypeptide of the present invention includes an isolated LP226, LP233, or LP236 polypeptide comprising at least one fragment, domain, or variant of at least 90-100% of the contiguous amino acids of at least one portion of at least one of SEQ ID NO:2, 4, or 6.

The present invention also provides an isolated LP226, LP233, or LP236 polypeptide as described herein, wherein the polypeptide further comprises at least one substitution, insertion, or deletion corresponding to portions or residues of at least one of SEQ ID NO:2, 4, or 6.

The present invention also provides an isolated nucleic acid probe, primer, or fragment, as described herein, wherein the nucleic acid comprises a polynucleotide of at least 10 nucleotides, corresponding or complementary to at least 10 nucleotides of at least one of SEQ ID NO:1, 3, 5.

A method of treatment or prophylaxis for disorders associated with loss of bone mineral content, osteopenia-associated disorders, proliferative disorders, cardiovascular disorders, neurological disorders, fibrotic disorders, disorders requiring wound or tissue repair, or disorders associated with human chromosomal region 11q14 can be effected with the polypeptides, nucleic acids, antibodies, vectors, host cells, transgenic cells, and/or compositions described. Accordingly, the present invention also includes methods for the prophylaxis or treatment of patho-physiological conditions in which at least one cell type involved in said condition is sensitive or responsive to a polypeptide, nucleic acid, antibody, host cell, transgenic cell, or composition of the present invention.

The present invention also provides a method for identifying compounds that bind an LP226, LP233, or LP236 polypeptide, comprising

a) admixing at least one isolated LP226, LP233, or LP236 polypeptide as described herein with a test compound or composition; and

b) detecting at least one binding interaction between the polypeptide and the compound or composition, optionally further comprising detecting a change in biological activity, such as a reduction or increase.

The present invention also provides an article of manufacture comprising a container, holding a composition effective for treating a disorder associated with loss of bone mineral content, an osteopenia-associated disorder, a proliferative disorder, a cardiovascular disorder, a neurological disorder, a fibrotic disorder, a disorder requiring wound or tissue repair, or a disorder associated with human chromosomal region 11q14; and a label.

### DETAILED DESCRIPTION OF THE PREFERRED EMBODIMENTS

Applicants have identified cDNA clones that encode novel polypeptides having sequence similarities with chordin. These include the following polypeptides and variants thereof:

1) Features of Polypeptides Encoded by LP226 Polynucleotides

LP226 polypeptides comprising the amino acid sequence of the open reading frame encoded by the polynucleotide sequence as shown in SEQ ID NO:1 are contemplated by the present invention. The isolated nucleic acid comprises DNA consisting of nucleotides 28 or about 109 through about 1315, inclusive, of SEQ ID NO:1.

TABLE 1
SEQ ID NO:1 (LP226).

| | |
|---|---|
| +1 | Met Val Pro Glu Val Arg Val |
| 1 | GAC CAG CGG CCT GAC CCT GGG GAA AGG ATG GTT CCC GAG GTG AGG GTC |
| | CTG GTC GCC GGA CTG GGA CCC CTT TCC TAC CAA GGG CTC CAC TCC CAG |
| +1 | Leu Ser Ser Leu Leu Gly Leu Ala Leu Leu Trp Phe Pro Leu Asp Ser |
| 49 | CTC TCC TCC TTG CTG GGA CTC GCG CTG CTC TGG TTC CCC CTG GAC TCC |
| | GAG AGG AGG AAC GAC CCT GAG CGC GAC GAG ACC AAG GGG GAC CTG AGG |
| +1 | His Ala Arg Ala Arg Pro Asp Met Phe Cys Leu Phe His Gly Lys Arg |
| 97 | CAC GCT CGA GCC CGC CCA GAC ATG TTC TGC CTT TTC CAT GGG AAG AGA |
| | GTG CGA GCT CGG GCG GGT CTG TAC AAG ACG GAA AAG GTA CCC TTC TCT |
| 1 | Tyr Ser Pro Gly Glu Ser Trp His Pro Tyr Leu Glu Pro Gln Gly Leu |
| 145 | TAC TCC CCC GGC GAG AGC TGG CAC CCC TAC TTG GAG CCA CAA GGC CTG |
| | ATG AGG GGG CCG CTC TCG ACC GTG GGG ATG AAC CTC GGT GTT CCG GAC |
| +1 | Met Tyr Cys Leu Arg Cys Thr Cys Ser Glu Gly Ala His Val Ser Cys |
| 193 | ATG TAC TGC CTG CGC TGT ACC TGC TCA GAG GGC GCC CAT GTG AGT TGT |
| | TAC ATG ACG GAC GCG ACA TGG ACG AGT CTC CCG CGG GTA CAC TCA ACA |
| 1 | Tyr Arg Leu His Cys Pro Pro Val His Cys Pro Gln Pro Val Thr Glu |
| 241 | TAC CGC CTC CAC TGT CCG GCT GTC CAC TGC CCC CAG CCT GTG ACG GAG |
| | ATG GCG GAG GTG ACA GGC GGA CAG GTG ACG GGG GTC GGA CAC TGC CTC |
| +1 | Pro Gln Gln Cys Cys Pro Lys Cys Val Glu Pro His Thr Pro Ser Gly |
| 289 | CCA CAG CAA TGC TGT CCC AAG TGT GTG GAA CCT CAC ACT CCC TCT GGA |
| | GGT GTC GTT ACG ACA GGG TTC ACA CAC CTT GGA GTG TGA GGG AGA CCT |
| +1 | Leu Arg Ala Pro Pro Lys Ser Cys Gln His Asn Gly Thr Met Tyr Gln |
| 337 | CTC CGG GCC CCA CCA AAG TCC TGC CAG CAC AAC GGG ACC ATG TAC CAA |
| | GAG GCC CGG GGT GGT TTC AGG ACG GTC GTG TTG CCC TGG TAC ATG GTT |
| +1 | His Gly Glu Ile Phe Ser Ala His Glu Leu Phe Pro Ser Arg Leu Pro |
| 385 | CAC GGA GAG ATC TTC AGT GCC CAT GAG CTG TTC CCC TCC CGC CTG CCC |
| | GTG CCT CTC TAG AAG TCA CGG GTA CTC GAC AAG GGG AGG GCG GAC GGG |
| +1 | Asn Gln Gys Val Leu Cys Ser Gys Thr Glu Gly Gln Ile Tyr Gys Gly |
| 433 | AAC CAG TGT GTC CTC TGC AGC TGC ACA GAG GGC CAG ATC TAC TGC GGC |
| | TTG GTC ACA CAG GAG ACG TCG ACG TGT CTC CCG GTC TAG ATG ACG CCG |
| +1 | Leu Thr Thr Cys Pro Glu Pro Gly Gys Pro Ala Pro Leu Pro Leu Pro |
| 481 | CTC ACA ACC TGC CCC GAA CCA GGC TGC CCA GCA CCC CTC CCG CTG CCA |
| | GAG TGT TGG ACG GGG CTT GGT CCG ACG GGT CGT GGG GAG GGC GAC GGT |
| +1 | Asp Ser Cys Cys Gln Ala Cys Lys Asp Glu Ala Ser Glu Gln Ser Asp |
| 529 | GAC TCC TGC TGC CAA GCC TGC AAA GAT GAG GCA AGT GAG CAA TCG GAT |
| | CTG AGG ACG ACG GTT CGG ACG TTT CTA CTC CGT TCA CTC GTT AGC CTA |
| +1 | Glu Glu Asp Ser Val Gln Ser Leu His Gly Val Arg His Pro Gln Asp |
| 577 | GAA GAG GAC AGT GTG CAG TCG CTC CAT GGG GTG AGA CAT CCT CAG GAT |
| | CTT CTC CTG TCA CAC GTC AGC GAG GTA CCC CAC TCT GTA GGA GTC CTA |
| +1 | Pro Cys Ser Ser Asp Ala Gly Arg Lys Arg Gly Pro Gly Thr Pro Ala |
| 625 | CCA TGT TCC AGT GAT GCT GGG AGA AAG AGA GGC CCG GGC ACC CCA GCC |
| | GGT ACA AGG TCA CTA CGA CCC TCT TTC TCT CCC GGC CCG TGG GGT CGG |
| 1 | Pro Thr Gly Leu Ser Ala Pro Leu Ser Phe Ile Pro Arg His Phe Arg |
| 673 | CCC ACT GGC CTC AGC GCC CCT CTG AGC TTC ATC CCT CGC CAC TTC AGA |
| | GGG TGA CCG GAG TCG CGG GGA GAC TCG AAG TAG GGA GCG GTG AAG TCT |
| +1 | Pro Lys Gly Ala Gly Ser Thr Thr Val Lys Ile Val Leu Lys Glu Lys |
| 721 | CCC AAG GGA GCA GGC AGC ACA ACT GTC AAG ATC GTC CTG AAG GAG AAA |
| | GGG TTC CCT CGT CCG TCG TGT TGA CAG TTC TAG CAG GAC TTC CTC TTT |
| 1 | His Lys Lys Ala Cys Val His Gly Gly Lys Thr Tyr Ser His Gly Glu |
| 769 | CAT AAG AAA GCC TGT GTG CAT GGC GGG AAG ACG TAC TCC CAC GGG GAG |
| | GTA TTC TTT CGG ACA CAC GTA CCG CCC TTC TGC ATG AGG GTG CCC CTC |
| +1 | Val Trp His Pro Ala Phe Arg Ala Phe Gly Pro Leu Pro Cys Ile Leu |
| 817 | GTG TGG CAC CCG GCC TTC CGT GCC TTC GGC CCC TTG CCC TGC ATC CTA |
| | CAC ACC GTG GGC CGG AAG GCA CGG AAG CCG GGG AAC GGG ACG TAG GAT |
| +1 | Cys Thr Cys Glu Asp Gly Arg Gln Asp Cys Gln Arg Val Thr Cys Pro |
| 865 | TGC ACC TGT GAG GAT GGC CGC CAG GAC TGC CAG CGT GTG ACC TGT CCC |
| | ACG TGG ACA CTC CTA CCG GCG GTC CTG ACG GTC GCA CAC TGG ACA GGG |
| +1 | Thr Glu Tyr Pro Cys Arg His Pro Glu Lys Val Ala Gly Lys Cys Cys |
| 913 | ACC GAG TAC CCC TGC CGT CAC CCC GAG AAA GTG GCT GGC AAG TGG TGC |
| | TGG CTC ATG GGG ACG GCA GTG GGG CTC TTT CAC CGA CCC TTC ACG ACG |
| +1 | Lys Ile Cys Pro Glu Asp Lys Ala Asp Pro Gly His Ser Glu Ile Ser |
| 961 | AAG ATT TGC CCA GAG GAC AAA GCA GAC CCT GGC CAC AGT GAG ATC AGT |
| | TTC TAA ACG GGT CTC CTG TTT CGT CTG GGA CCG GTG TCA CTC TAG TCA |
| +1 | Ser Thr Arg Cys Pro Lys Ala Pro Gly Arg Val Leu Val His Thr Ser |
| 1009 | TCT ACC AGG TGT CCC AAG GCA CCG GGC CGG GTC CTC GTC CAC ACA TCG |
| | AGA TGG TCC ACA GGG TTC CGT GGC CCG GCC CAG GAG CAG GTG TGT AGC |
| +1 | Val Ser Pro Ser Pro Asp Asn Leu Arg Arg Phe Ala Leu Glu His Glu |
| 1057 | GTA TCC CCA AGC CCA GAC AAC CTG CGT CGC TTT GCC CTG GAA CAC GAG |
| | CAT AGG GGT TCG GGT CTG TTG GAC GCA GCG AAA CGG GAC CTT GTG CTC |
| 1 | Ala Ser Asp Leu Val Glu Ile Tyr Leu Trp Lys Leu Val Lys Cly Ile |
| 1105 | GCC TCG GAC CTC GTG GAG ATC TAC CTC TGG AAC CTG GTA AAA GGA ATC |
| | CGG AGC CTG GAC GAC CTC TAG ATG GAG ACC TTC GAC CAT TTT CCT TAG |
| +1 | Phe His Leu Thr Gln Ile Lys Lys Val Arg Lys Gln Asp Phe Gln Lys |
| 1153 | TTC CAC TTG ACT CAG ATC AAG AAA GTC AGG AAG CAA GAC TTC CAG AAA |
| | AAG GTG AAC TGA GTC TAG TTC TTT GAG TCC TTC GTT CTG AAG GTC TTT |
| +1 | Glu Ala Gln His Phe Arg Leu Leu Ala Gly Pro His Glu Gly His Trp |
| 1201 | GAG GCA CAG CAC TTC CGA CTG CTC GCT GGC CCC CAC GAA GGT CAC TGG |
| | CTC CGT GTC GTG AAG GCT GAC GAG CGA CCG GGG GTG CTT CCA GTG ACC |
| +1 | Asn Val Phe Leu Ala Gln Thr Leu Glu Leu Lys Val Thr Ala Ser Pro |
| 1249 | AAC GTC TTC CTA GCC CAG ACG CTG GAG GTG AAG GTC ACG GCC AGT CCA |
| | TTG CAG AAG GAT CGG GTC TGG GAC CTC GAC TTC CAG TGC CGG TCA GGT |
| +1 | Asp Lys Val Thr Lys Thr *** |
| 1297 | GAC AAA GTG ACC AAG ACA TAA CAA AGA CGT AAC AGT TGC AGA TAT GAG |
| | CTG TTT CAC TGG TTC TGT ATT GTT TCT GGA TTG TCA ACG TCT ATA CTC |
| 1345 | CTG TAT AAT TGT TGT TAT TAT ATA TTA ATA AAT AAG AAG TTG CAT TAC |
| | GAC ATA TTA ACA ACA ATA ATA TAT AAT TAT TTA TTC TTC AAC GTA ATG |
| 1393 | CGT CAA AAA AAA A |
| | GGA GTT TTT TTT T |

In one embodiment, the invention provides isolated nucleic acid molecules comprising DNA encoding the LP226 polypeptides. In another aspect, the isolated nucleic acid comprises DNA encoding LP226 having amino acid residues from 1 or about 27 to about 430, inclusive, of SEQ ID NO:2, or that are complementary to such encoding nucleic acid sequences, and remain stably bound to them under at least moderate, and optionally, high stringency conditions. Specifically, polypeptides of the present invention comprise the amino acid sequence as shown in SEQ ID NO:2, as well as fragments, variants, and derivatives thereof.

TABLE 2
SEQ ID NO:2 (LP226).

MVPEVRVLSSLLGLALLWFPLDSHARARPDMFCLFHGKRYSPGESWHPYL
EPQGLMYCLRCTCSEGAHVSCYRLHCPPVHCPQPVTEPQQCCPKCVEPHT
PSGLRAPPKSCQHNGTMYQHGEIFSAHELFPSRLPNQCVLCSCTEGQTYC
GLTTCPEPGCPAPLPLPDSCCQACKDEASEQSDEEDSVQSLHGVRHPQDP
CSSDAGRKRGPGTPAPTGLSAPLSFIPRHFRPKGAGSTTVKIVLKEKHKK
ACVHGGKTYSHGEVWHPAFRAFGPLPCILCTCEDGRQDCQRVTCPTEYPC
RHPEKVAGKCCKICPEDKADPGHSEISSTRCPKAPGRVLVHTSVSPSPDN
LRRFALEHEASDLVEIYLWKLVKGIFHLTQIKKVRKQDFQKEAQHFRLLA
GPHEGHWNVFLAQTLETJKVTASPDKVTKT

LP226 polypeptide shares sequence similarity with human chordin; both polypeptides contain cysteine-rich motifs which repeat throughout the sequence. The human chordin repeating motif is CX₂₄CX₂CX₁CX₆₋₈CX₄CX₄CX₁₂CCX₂CX₃R/K. LP226 has the following repeating motif: CX₂₄₋₂₆CX₂CX₁₋₂CX₆₋₇CX₄CX₄₋₅CX₉CCX₂CX_{4-end}. Refer to Tables 7 and 8 for motif and sequence comparisons.

In contrast to human chordin, LP226 does not contain known tolloid cleavage sites that are present in wild type chordin. Tolloid cleaves human chordin at the N-terminus at HRSYS . . . DRGEP and at the C-terminus at DPMQA . . . DGPRG. Scott, et al., Dev. Biol. 213(2): 283-300 (1999). That cleavage renders chordin inactive and unable to bind BMPs. Since this cleavage site is not found in LP226, it cannot be cleaved by tolloid and, therefore, cannot be inactivated.

Because LP226 contains the cysteine-rich motifs and lacks the tolloid cleavage site, LP226 is able to bind BMPs and other TGF-betas. Accordingly, compositions comprising LP226 polypeptides and polynucleotides are useful for the diagnosis, treatment, and intervention of proliferative disorders, such as osteosarcoma, breast and prostate cancer, which may be metastasized by BMPs. Additionally, compositions comprising LP226 polypeptides and polynucleotides are useful for the diagnosis, treatment, and intervention of cardiovascular disorders, fibrotic disorders, and neurological disorders. Moreover, compositions comprising LP226 antibodies or antagonists are useful for the diagnosis, treatment, and intervention of disorders which are ameliorated by TGF-betas or BMPs. These include, but are not limited to, disorders requiring wound and tissue repair, disorders associated with loss of bone mineral density, and osteopenia-related disorders. Antagonists to LP226 inhibit its ability to bind TGF-betas or BMPs, thereby allowing TGF-beta or BMP activities to occur.

2) Features of Polypeptides Encoded by LP233 Polynucleotides

LP233 polypeptides comprising the amino acid sequence of the open reading frame encoded by the polynucleotide sequence as shown in SEQ ID NO:3 are contemplated by the present invention. The isolated nucleic acid comprises DNA consisting of nucleotides 234 or about 315 through about 720, inclusive, of SEQ ID NO:3.

TABLE 3
SEQ ID NO:3 (LP233).

| | |
|---|---|
| 1 | CTC TCC CTC CTT TCC CGC GTT CTC TTT CCA CCT TTC TCT TCT TCC CAC |
| | GAG AGG GAG GAA AGG GCG CAA GAG AAA GGT GGA AAG AGA AGA AGG GTG |
| 49 | CTT AGA CCT CCC TTC CTG CCC TCC TTT CCT GCC CAC TGC TGC TTC CTG |
| | GAA TCT GGA GGG AAG GAC GGG AGG AAA GGA CGG GTG ACG ACG AAG GAC |
| 97 | GCC CTT CTC CGA CCC CGC TCT AGC AGC AGA CCT CCT GGG GTC TGT GGG |
| | CGG GAA GAG GCT GGG GCG AGA TCG TCG TCT GGA GGA CCC CAG ACA CCC |
| 145 | TTG ATC TGT GGC CCC TGT GCC TCC GTG TCC TTT TCG TCT CCC TTC CTC |
| | AAC TAG ACA CCG GGG ACA CGG AGG CAC AGG AAA AGC AGA GGG AAG GAG |
| +1 | Met Val |
| 193 | CCG ACT CCG CTC CCG GAC CAG CGG CCT GAC CCT GGG GAA AGG ATG GTT |
| | GGC TGA GGC GAG GGC CTG GTC GCC GGA CTG GGA CCC CTT TCC TAC CAA |
| +1 | Pro Glu Val Arg Val Leu Ser Ser Leu Leu Gly Leu Ala Leu Leu Trp |
| 241 | CCC GAG GTG AGG GTC CTC TCC TCC TTG CTG GGA CTC GCG CTG CTC TGG |
| | GGG CTC CAC TCC CAG GAG AGG AGG AAC GAC CCT GAG CGC GAC GAG ACC |
| 1 | Phe Pro Leu Asp Ser His Ala Arg Ala Arg Pro Asp Met Phe Cys Leu |
| 289 | TTC CCC CTG GAC TCC CAC GCT CGA GCC CGC CCA GAG ATG TTC TGC CTT |
| | AAG GGG GAC CTG AGG GTG CGA GCT CGG GCG GGT CTG TAC AAG ACG GAA |
| 1 | Phe His Gly Lys Arg Tyr Ser Pro Gly Glu Ser Trp His Pro Tyr Leu |
| 337 | TTC CAT GGG AAG AGA TAC TCC CCC GGG GAG AGG TGG CAC CCC TAG TTG |
| | AAG GTA CCC TTC TCT ATG AGG GGG CCG CTC TCG ACC GTG GGG ATG AAC |
| +1 | Glu Pro Gln Gly Leu Met Tyr Cys Leu Arg Cys Thr Cys Ser Glu Gly |
| 385 | GAG CCA GAA GGC CTG ATG TAC TGC CTG CGC TGT ACC TGC TCA GAG GGC |
| | CTC GGT GTT CCG GAC TAC ATG ACG GAC GCG ACA TGG ACG AGT CTC CCG |
| +1 | Ala His Val Ser Cys Tyr Arg Leu His Cys Pro Pro Val His Gys Pro |
| 433 | GCC CAT GTG AGT TGT TAC CGC CTC CAC TGT CCG CCT GTC CAC TGC CCC |
| | CGG GTA CAC TCA ACA ATG GCG GAG GTG ACA GGC GGA CAG GTG ACG GGG |
| +1 | Gln Pro Val Thr Glu Pro Gln Gln Cys Cys Pro Lys Cys Val Glu Pro |
| 481 | CAG CCT GTG ACG GAG CCA CAG CAA TGC TGT CCC AAG TGT GTG GAA CCT |
| | GTC GGA CAC TGC CTC GGT GTG GTT ACG ACA GGG TTC ACA CAC CTT GGA |
| +1 | His Thr Pro Ser Gly Leu Arg Ala Pro Pro Lys Ser Cys Gln His Asn |
| 529 | CAC ACT CCC TCT GGA CTC CGG GCC CCA CCA AAG TCC TGC CAG CAC AAC |
| | GTG TGA GGG AGA GGT GAG GCC CGG GGT GGT TTC AGG AGG GTC GTG TTG |
| +1 | Gly Thr Met Tyr Gln His Gly Glu Ile Phe Ser Ala His Glu Leu Phe |
| 577 | GGG ACC ATG TAC CAA CAC GGA GAG ATC TTC AGT GCC CAT GAG CTG TTC |
| | CCC TGG TAC ATG GTT GTG CCT CTC TAG AAG TCA CGG GTA CTC GAC AAG |
| +1 | Pro Ser Arg Leu Pro Asn Gln Gys Val Leu Gys Ser Cys Thr Met Arg |
| 625 | CCC TCC CGC CTG CCC AAC CAG TGT GTC CTC TGC AGC TGC ACA ATG AGG |
| | GGG AGG GCG GAG GGG TTG GTC ACA CAG GAC ACG TCG ACG TGT TAC TCC |
| +1 | Gln Val Ser Asn Arg Met Lys Arg Thr Val Cys Ser Arg Ser Met Gly |
| 673 | CAA GTG AGC AAT CGG ATG AAG AGG ACA GTG TGC AGT CGC TCC ATG GGG |
| | GTT CAC TCG TTA GCC TAC TTC TCC TGT CAC ACG TCA GCG AGG TAC CCC |
| +1 | *** |
| 721 | TGA GAC ATC CTC AGG ATC CAT GTT CCA GTG ATG CTG GGA CGA AGA GAG |
| | ACT CTG TAG GAG TCC TAG GTA CAA GGT GAC TAC GAC CCT CTT TGT CTC |
| 769 | GCC CGG GCA CCC CAG CCC CCA CTG GCC TCA GCG CCC CTC TGA GCT TCA |
| | CGG GCC GGT GGG GTC GGG GGT GAC CGG AGT CGC GGG GAG ACT GGA AGT |
| 817 | TCC CTC GCC ACT TCA GAC CCA AGG GAG CAG GCA GCA CAA CTG TCA AGA |
| | AGG GAG CGG TGA AGT CTG GGT TCC GTC GTC CGT CGT GTT GAC AGT TCT |
| 865 | TCG TCC TGA AGG AGA AAC ATA AGA AAG CCT GTG TGC ATG GCG GGA AGA |
| | AGC AGG ACT TCC TCT TTG TAT TCT TTC GGA CAC ACG TAC CGC CCT TCT |
| 913 | CGT ACT CCC ACG GGG AGG TGT GGC ACC CGG CCT TCC GTG CCT TCG GCC |
| | GCA TGA GGG TGC CCC TCC ACA CCG TGG GCC GGA AGG CAC GGA AGC CGG |
| 961 | CCT TGC CCT GCA TCC TAT GCA CCT GTG AGG ATG GCC GCC AGG ACT GCC |
| | GGA ACG GGA CGT AGG ATA CGT GGA CAC TCC TAC CGG CGG TCC TGA CGG |
| 1009 | AGC GTG TGA CCT GTC CCA CCG AGT ACC CCT GCC GTC ACC CCG AGA AAG |
| | TCG CAC ACT CGA CAG GGT GGC TCA TGG GGA CGG CAG TGG GGC TCT TTC |
| 1057 | TGG CTG GGA AGT GCT GOA AGA TTT GCC CAG AGG ACA AAG CAG ACC CTG |
| | ACC GAC CCT TCA CGA CGT TCT AAA CGG GTC TCC TGT TTC GTC TGG GAC |
| 1105 | GCC ACA GTG AGA TCA GTT CTA CCA GGT GTC CCA AGG CAC CGG GCC GGG |
| | CGG TGT CAC TCT AGT CAA GAT GGT CCA CAG GGT TCC GTG GCC CGG CCC |
| 1153 | TCC TCG TCC ACA CAT CGG TAT CCC CAA GCC CAG ACA ACC TGC GTC GCT |
| | AGG AGC AGG TGT GTA GCC ATA GGG GTT CGG GTC TGT TGG ACG CAG CGA |
| 1201 | TTG CCC TGG AAC ACG AGG CCT CGG ACT TGG TGG AGA TCT ACC TCT GGA |
| | AAC GGG ACC TTG TGC TCC GGA GCC TGA ACC ACC TCT AGA TGG AGA CCT |
| 1249 | AGC TGG TAA AAG GAA TCT TCC ACT TGA CTC AGA TCA AGA AAG TCA GGA |
| | TCG ACC ATT TTC CTT AGA AGG TGA ACT GAG TCT AGT TCT TTC AGT CCT |
| 1297 | AGC AAG ACT TCC AGA AAG AGG CAC AGC ACT TCC GAC TGC TCG CTG GCC |
| | TCG TTC TGA AGG TCT TTC TCC GTG TCG TGA AGG CTG ACG AGC GAC CGG |
| 1345 | CCC ACG AAG GTC ACT GGA ACG TCT TCC TAG CCC AGA CCC TGG AGC TGA |
| | GGG TGC TTC CAG TGA CCT TGC AGA AGG ATC GGG TCT GGG ACC TCG ACT |
| 1393 | AGG TCA CGG CCA GTC CAG ACA AAG TGA CCA AGA CAT AAC AAA GAC CTA |
| | TCC AGT GCC GGT CAG GTC TGT TTC ACT GGT TCT GTA TTG TTT CTG GAT |
| 1441 | ACA GTT GCA GAT ATG AGC TGT ATA ATT GTT GTT ATT ATA TAT TAA TAA |
| | TGT CAA CGT CTA TAC TCG ACA TAT TAA CAA CAA TAA TAT ATA ATT ATT |
| 1489 | ATA AGA AGT TGC ATT ACC CTC AAA AAA AA |
| | TAT TCT TCA ACG TAA TGG GAG TTT TTT TT |

In one embodiment, the invention provides isolated nucleic acid molecules comprising DNA encoding the LP233 polypeptides. In another aspect, the isolated nucleic acid comprises DNA encoding LP233 having amino acid residues from 1 or about 27 to about 162, inclusive, of SEQ ID NO:4, or that are complementary to such encoding nucleic acid sequences, and remain stably bound to them under at least moderate, and optionally, high stringency conditions. Specifically, polypeptides of the present invention comprise the amino acid sequence as shown in SEQ ID NO:4, as well as fragments, variants, and derivatives thereof.

TABLE 4
SEQ ID NO:4 (LP233).

MVPEVRVLSSLLGLALLWFPLDSHARARPDMFCLFHGKRYSPGESWHPYL
EPOGLMYCLRCTCSEGAHVSCYRLHCPPVHCPQPVTEPQQCCPKCVEPHT
PSGLRAPPKSCQHNGTMYQHGEIFSAHELFPSRLPNQCVLCSCTMRQVSN
RMKRTVCSRSMG

LP233 polypeptide shares sequence similarity with human chordin; both polypeptides contain cysteine-rich motif in the sequence. The human chordin repeating motif is CX₂₄CX₂CX₁CX₆₋₈CX₄CX₄CX₁₂CCX₂CX₃R/K. LP233 has the following motif: CX₂₄CX₂CX₁CX₇CX₄CX₄CX₉CCX₂R/. Refer to Tables 7 and 8 for motif and sequence comparisons. LP233 may be an alternatively spliced variant of LP226.

In contrast to human chordin, LP233 does not contain known tolloid cleavage sites that are present in wild type chordin. Tolloid cleaves human chordin at the N-terminus at HRSYS . . . DRGEP and at the C-terminus at DPMQA . . . DGPRG. Scott, et al., Dev. Biol. 213(2): 283-300 (1999). That cleavage renders chordin inactive and unable to bind BMPs. Since this cleavage site is not found in LP233, it cannot be cleaved by tolloid and, therefore, cannot be inactivated.

Because LP233 contains the cysteine-rich motif and lacks the tolloid cleavage site, LP233 is able to bind BMPs and other TGF-betas. Accordingly, compositions comprising LP233 polypeptides and polynucleotides are useful for the diagnosis, treatment, and intervention of proliferative disorders, such as osteosarcoma, breast and prostate cancer, which may be metastasized by BMPs. Additionally, compositions comprising LP233 polypeptides and polynucleotides are useful for the diagnosis, treatment, and intervention of cardiovascular disorders, fibrotic disorders, and neurological disorders. Moreover, compositions comprising LP233 antibodies or antagonists are useful for the diagnosis, treatment, and intervention of disorders which are ameliorated by TGF-betas or BMPs. These include, but are not limited to, disorders requiring wound and tissue repair, disorders associated with loss of bone mineral density, and osteopenia-related disorders. Antagonists to LP233 inhibit its ability to bind TGF-betas or BMPs, thereby allowing TGF-beta or BMP activities to occur.

3) Features of Polypeptides Encoded by LP236 Polynucleotides

LP236 polypeptides comprising the amino acid sequence of the open reading frame encoded by the polynucleotide sequence as shown in SEQ ID NO:5 are contemplated by the present invention. The isolated nucleic acid comprises DNA consisting of nucleotides 307 or about 387 through about 1660, inclusive, of SEQ ID NO:5.

TABLE 5
SEQ ID NO:5 (12236).

| | |
|---|---|
| 1 | CGG GTC GAC CCA CGC GTC CCC CCA CGC GTC CGC CCC TCT CCC TTC TGC |
| | GCC CAG CTG GGT GCG CAG GCG GGT GCG CAG GCG CGG AGA GGG AAG ACG |
| 49 | TGG ACC TTC CTT CGT CTC TCC ATC TCT CCC TCC TTT CCC CGC GTT CTC |
| | ACC TGG AAG GAA GCA GAG AGG TAG AGA GGG AGG AAA GGG GCG CAA GAG |
| 97 | TTT CCA CCT TTC TCT TCT TCC CAC CTT AGA CCT CCC TTC CTG CCC TCC |
| | AAA GGT GGA AAG AGA AGA AGG GTG GAA TCT GGA GGG AAG GAC GGG AGG |
| 145 | TTT CCT GCC CAC CGC TGC TTC CTG GCC CTT CTC CGA CCC CGC TCT AGC |
| | AAA GGA CGG GTG GCG ACG AAG GAC CGG GAA GAG GCT GGG GCG AGA TCG |
| 193 | AGC AGA CCT CCT GGG GTC TGT GGG TTG ATC TGT GGC CCC TGT GCC TCC |
| | TCG TCT GGA GGA CCC CAG ACA CCC AAC TAG ACA CCG GGG ACA CGG AGG |
| 241 | GTG TCC TTT TCG TCT CCC TTC CTC CCG ACT CCG CTC CCG GAC CAG CGG |
| | CAC AGG AAA AGO AGA GGG AAG GAG GGC TGA GGC GAG GGC CTG GTC GCC |
| +1 | Met Val Pro Glu Val Arg Val Leu Ser Ser |
| 289 | CCT GAC CCT GGG GAA AGG ATG GTT CCC GAG GTG AGG GTC CTC TCC TCC |
| | GGA CTG GGA CCC CTT TCC TAC CAA GGG CTC CAC TCC CAG GAG AGG AGG |
| +1 | Leu Leu Gly Leu Ala Leu Leu Trp Phe Pro Leu Asp Ser His Ala Arg |
| 337 | TTG CTG GGA CTC GCG CTG CTC TGG TTC CCC CTG GAO TCC CAC GCT CGA |
| | AAC GAC CCT GAG CGC GAC GAG ACC AAG GGG GAC CTG AGG GTG CGA GCT |
| +1 | Ala Arg Pro Asp Met Phe Cys Leu Phe His Gly Lys Arg Tyr Ser Pro |
| 385 | GCC CGC CCA GAC ATG TTC TGC CTT TTC CAT GGG AAG AGA TAC TCC CCC |
| | CGG GCG GGT CTG TAC AAG ACG GAA AAG GTA CCC TTC TCT ATG AGG GGG |
| 1 | Gly Glu Ser Trp His Pro Tyr Leu Gln Pro Gln Gly Leu Met Tyr Cys |
| 433 | GGC GAG AGC TGG CAC CCC TAC TTG GAG CCA CAA GGC CTG ATG TAC TGC |
| | CCG CTC TCG ACC GTG GGG ATG AAC CTC GGT GTT CCG GAC TAC ATG ACG |
| +1 | Leu Arg Cys Thr Cys Ser Glu Gly Ala His Val Ser Cys Tyr Arg Leu |
| 481 | CTG CGC TGT ACC TGC TCA GAG GGC GCC CAT GTG AGT TGT TAC CGC CTC |
| | GAC GCG ACA TGG ACG AGT CTC CCG CGG GTA CAC TCA ACA ATG GCG GAG |
| +1 | His Cys Pro Pro Val His Cys Pro Gln Pro Val Thr Glu Pro Gln Gln |
| 529 | CAC TGT CCG CCT GTC CAC TGC CCC CAG CCT GTG ACG GAG CCA CAG CAA |
| | GTG ACA GGC GGA CAG GTG ACG GGG GTC GGA CAC TGC CTC GGT GTC GTT |
| +1 | Cys Cys Pro Lys Cys Val Glu Pro His Thr Pro Ser Gly Leu Arg Ala |
| 577 | TGC TGT CCC AAG TGT GTG GAA CCT CAC ACT CCC TCT GGA CTC CGG GCC |
| | ACG ACA GGG TTC ACA CAC CTT GGA GTG TGA GGG AGA CCT GAG GCC CGG |
| +1 | Pro Pro Lys Ser Cys Gln His Asn Cly Thr Met Tyr Gln His Gly Glu |
| 625 | CCA CCA AAG TCC TGC CAG CAC AAC GGG ACC ATG TAC CAA CAC GGA GAG |
| | GGT GGT TTC AGG ACG GTC GTG TTG CCC TGG TAC ATG GTT GTG CCT CTC |
| +1 | Ile Phe Ser Ala His Glu Leu Phe Pro Ser Arg Leu Pro Asn Gln Cys |
| 673 | ATC TTC AGT GCC CAT GAG CTG TTC CCC TCC CGC CTG CCC AAC CAG TGT |
| | TAG AAG TCA CGG GTA CTC GAC AAG GGG AGG GCG GAC GGG TTG GTC ACA |
| +1 | Val Len Cys Ser Cys Thr Glu Gly Gln Ile Tyr Cys Gly Leu Thr Thr |
| 721 | GTC CTC TGC AGC TGC ACA GAG GGC CAG ATC TAC TGC GGC CTC ACA ACC |
| | CAG GAG ACG TCG ACG TGT CTC CCG GTC TAG ATG ACG CCG GAG TGT TGG |
| +1 | Cys Pro Gln Pro Gly Cys Pro Ala Pro Leu Pro Leu Pro Asp Ser Cys |
| 769 | TGC CCC GAA CCA GGC TGC CCA GCA CCC CTC CCA CTG CCA GAC TCC TGC |
| | ACG GGG CTT GGT CCG ACG GGT CGT GGG GAG GGT GAC GGT CTG AGG ACG |
| +1 | Cys Gln Ala Cys Lys Asp Glu Ala Ser Glu Gln Ser Asp Glu Glu Asp |
| 817 | TGC CAA GCC TGC AAA GAT GAG GCA AGT GAG CAA TCG GAT GAA GAG GAC |
| | ACG GTT CGG ACG TTT CTA CTC CGT TCA CTC GTT AGC CTA CTT CTC CTG |
| +1 | Ser Val Gln Ser Leu His Gly Val Arg His Pro Gln Asp Pro Cys Ser |
| 865 | AGT GTG CAG TCG CTC CAT GGG GTG AGA CAT CCT CAG GAT CCA TGT TCC |
| | TCA CAC GTC AGC GAG CTA CCC CAC TCT GTA GCA GTC CTA GGT ACA AGG |
| +1 | Ser Asp Ala Gly Arg Lys Arg Gly Pro Cly Thr Pro Ala Pro Thr Cly |
| 913 | AGT GAT GCT GGG AGA AAG AGA CCC CCG CCC ACC CCA GCC CCC ACT GGC |
| | TCA CTA CGA CCC TCT TTC TCT CCC CCC CCC TGG GGT CGG GGG TGA CCG |
| +1 | Leu Ser Ala Pro Leu Ser Phe Ile Pro Arg His Phe Arg Pro Lys Gly |
| 961 | CTC AGC GCC CCT CTG AGC TTC ATC CCT CGC CAC TTC AGA CCC AAG GGA |
| | GAG TCG CCC CGA GAC TCC AAC TAG GGA CCC CTG AAG TCT CCC TTC CCT |
| +1 | Ala Gly Ser Thr Thr Val Lys Ile Val Leu Lys Glu Lys His Lys Lys |
| 1009 | GCA GGC AGC ACA ACT GTC AAG ATC GTC CTG AAG GAG AAA CAT AAG AAA |
| | CGT CCC TCG TGT TGA CAG TTC TAG CAG GAC TTC CTC TTT GTA TTC TTT |
| +1 | Ala Cys Val His Gly Gly Lys Thr Tyr Ser His Gly Glu Val Trp His |
| 1057 | GCC TGT GTG CAT GGC GGG AAG ACG TAG TCC CAC GGG GAG GTG TGG CAC |
| | CGG ACA CAC GTA CCG CCC TTC TGC ATG AGG GTG CCC CTC CAC ACC GTG |
| +1 | Pro Ala Phe Arg Ala Phe Gly Pro Leu Pro Cys Ile Leu Cys Thr Cys |
| 1105 | CCG GCC TTC CGT GCC TTC GGC CCC TTG CCC TGC ATC CTA TGC ACC TGT |
| | GGC CGG AAG GCA CGG AAG CCG GGG AAC GGG ACG TAG GAT ACG TGG ACA |
| +1 | Glu Asp Gly Arg Gln Asp Cys Gln Arg Val Thr Cys Pro Thr Glu Tyr |
| 1153 | GAG GAT GGC CGC CAG GAC TGC CAG CGT GTG ACC TGT CCC ACC GAG TAC |
| | CTC CTA CCG GCG GTC CTG ACG GTC GCA CAC TGG ACA GGG TGG CTC ATG |
| 1 | Pro Cys Arg His Pro Glu Lys Val Ala Gly Lys Cys Cys Lys Ile Cys |
| 1201 | CCC TGC CGT CAC CCC GAG AAA GTG GCT GGG AAG TGC TGC AAG ATT TGC |
| | GGG ACG GCA GTG GGG CTC TTT CAC CGA CCC TTC ACG ACG TTC TAA ACG |
| +1 | Pro Glu Asp Lys Ala Asp Pro Gly His Ser Glu Ile Ser Ser Thr Arg |
| 1249 | CCA GAG CAC AAA GCA GAC CCT GGC CAC AGT GAG ATC AGT TCT ACC AGG |
| | GGT CTC CTG TTT CGT CTG GGA CCG GTG TCA CTC TAG TCA AGA TGG TCC |
| +1 | Cys Pro Lys Ala Pro Gly Arg Val Leu Val His Thr Ser Val Ser Pro |
| 1297 | TGT CCC AAG GCA CCG GGC CGG GTC CTC GTC CAC ACA TCG GTA TCC CCA |
| | ACA GGG TTC CGT GGC CCG GCC CAG GAG CAG GTG TGT AGC CAT AGG GGT |
| +1 | Ser Pro Asp Asn Leu Arg Arg Phe Ala Leu Glu His Glu Ala Ser Asp |
| 1345 | AGC CCA GAC AAC CTG CGT CGC TTT GCC CTG GAA CAC GAG GCC TCG GAC |
| | TCG GGT CTG TTG GAC GCA GCG AAA CGG GAC CTT GTG CTC CGG AGC CTG |
| +1 | Leu Val Glu Ile Tyr Leu Trp Lys Leu Val Lys Asp Glu Glu Thr Glu |
| 1393 | TTG GTG GAG ATC TAC CTC TGG AAG CTG GTA AAA GAT GAG GAA ACT GAG |
| | AAC CAC CTC TAG ATG GAG ACC TTC GAC CAT TTT CTA CTC CTT TGA CTC |
| +1 | Ala Gln Arg Guy Glu Val Pro Gly Pro Arg Pro His Ser Gln Asn Leu |
| 1441 | GCT CAG AGA GGT GAA GTA CCT GGC CCA AGG CCA CAC AGC CAG AAT CTT |
| | CGA GTC TCT CCA CTT CAT GGA CCG GGT TCC GGT GTG TCG GTC TTA GAA |
| +1 | Pro Leu Asp Ser Asp Gln Glu Ser Gln Gln Ala Arg Leu Pro Glu Arg |
| 1489 | CCA CTT GAC TCA GAT CAA GAA AGT CAG GAA GCA AGA CTT CCA GAA AGA |
| | GGT GAA CTG ACT CTA GTT CTT TCA GTC CTT CGT TCT GAA GGT CTT TCT |
| +1 | Gly Thr Ala Len Pro Thr Ala Ary Trp Pro Pro Arg Arg Ser Leu Gln |
| 1537 | GGC ACA GCA CTT CCG ACT GCT CGC TGG CCC CCA CGA AGG TCA CTG GAA |
| | CCG TGT CGT GAA GGC TGA CGA GCG ACC GGG GGT GCT TCC AGT GAC CTT |
| +1 | Arg Leu Pro Ser Pro Asp Pro Gly Ala Glu Gly His Gly Gln Ser Arg |
| 1585 | CGT CTT CCT AGC CCA GAC CCT GGA GCT GAA GGT CAC GGC CAG TCC AGA |
| | GCA GAA GGA TCG GGT CTG GGA CCT CGA CTT CCA GTG CCG GTC AGG TCT |
| +1 | Gln Ser Asp Gln Asp Ile Thr Lys Thr *** |
| 1633 | CAA ACT GAC CAA GAC ATA ACA AAG ACC TAA CAG TTG CAG ATA TGA GCT |
| | GTT TCA CTG GTT CTG TAT TGT TTC TGG ATT GTC AAC GTC TAT ACT CGA |
| 1681 | GTA TAA TTG TTG TTA TTA TAT ATT AAT AAA TAA GAA GTT GCA TTA CCC |
| | CAT ATT AAC AAC AAT AAT ATA TAA TTA TTT ATT CTT CAA CGT AAT GGG |
| 1729 | TCA AAA AAA A |
| | AGT TTT TTT T |

In one embodiment, the invention provides isolated nucleic acid molecules comprising DNA encoding the LP236 polypeptides. In another aspect, the isolated nucleic acid comprises DNA encoding LP236 having amino acid residues from 1 or about 27 to about 451, inclusive, of SEQ ID NO:6, or that are complementary to such encoding nucleic acid sequences, and remain stably bound to them under at least moderate, and optionally, high stringency conditions. Specifically, polypeptides of the present invention comprise the amino acid sequence as shown in SEQ ID NO:6, as well as fragments, variants, and derivatives thereof.

TABLE 6
SEQ ID NO:6 (LP236).

MVPEVRVLSSLLGLALLWFPLDSHARARPDMFCLFHGKRYSPGESWHPYL
EPQGLMYCLRCTCSEGAHVSCYRLHCPPVHCPQPVTEPQQCCPKCVEPHT
PSGLRAPPKSCQHNGTMYQHGEIFSAHELFPSRLPNQCVLCSCTEGQIYC
GLTTCPEPGCPAPLPLPDSCCQACKDEASEQSDEEDSVQSLHGVRHPQDP
CSSDAGRKRGPGTPAPTGLSAPLSFIPRHFRPKGAGSTTVKIVLKEKHKK
ACVHGGKYTSHGEVWHPAFRAFGPLPCILCTCEDGRQDCQRVTCPTEYPC
RHPEKVAGKCCKICPEDKADPGHSEISSTRCPKAPGRVLVHTSVSPSPDN
LRRFALEHEASDLVEIYLWKLVKDEETEAQRGEVPGPRPHSQNLPLDSDQ
ESQEARLPERGTALPTARWPPRRSLERLPSPDPGAEGHGQSRQSDQDITK
T

LP236 polypeptide shares sequence similarity with human chordin; both polypeptides contain cysteine-rich motifs which repeat throughout the sequence. The human chordin repeating motif is CX₂₄CX₂CX₁CX₆₋₈CX₄CX₄CX₁₂CCX₂CX₃R/K. LP236 has the following repeating motif: CX₂₄₋₂₆CX₂CX₁CX₆₋₇CX₄CX₄₋₅CX₉CCX₂CX_{3-end}. Refer to Tables 7 and 8 for motif and sequence comparisons. LP236 may be an alternatively spliced variant of LP226.

In contrast to human chordin, LP236 does not contain known tolloid cleavage sites that are present in wild type chordin. Tolloid cleaves human chordin at the N-terminus at HRSYS . . . DRGEP and at the C-terminus at DPMQA . . . DGPRG. Scott, et al., Dev. Biol. 213(2): 283-300 (1999). That cleavage renders chordin inactive and unable to bind BMPs. Since this cleavage site is not found in LP236, it cannot be cleaved by tolloid and, therefore, cannot be inactivated.

Because LP236 contains the cysteine-rich motifs and lacks the tolloid cleavage site, LP236 is able to bind BMPs and other TGF-betas. Accordingly, compositions comprising LP236 polypeptides and polynucleotides are useful for the diagnosis, treatment, and intervention of proliferative disorders, such as osteosarcoma, breast and prostate cancer, which may be metastasized by BMPs. Additionally, compositions comprising LP236 polypeptides and polynucleotides are useful for the diagnosis, treatment, and intervention of cardiovascular disorders, fibrotic disorders, and neurological disorders. Moreover, compositions comprising LP236 antibodies or antagonists are useful for the diagnosis, treatment, and intervention of disorders which are ameliorated by TGF-betas or BMPs. These include, but are not limited to, disorders requiring wound and tissue repair, disorders associated with loss of bone mineral density, and osteopenia-related disorders. Antagonists to LP236 inhibit its ability to bind TGF-betas or BMPs, thereby allowing TGF-beta or BMP activities to occur.

TABLE 7
Human chordin. LP226, LP233, and LP236 sequence alignment.

| | |
|---|---|
| a | human CHORDIN |
| b | LP226 |
| c | TP233 |
| d | LP236 |

| | |
|---|---|
| 1a | MPSLPAPPAPLLLLGLLLLGSRPARGAGPEPPVLPIRSEKEPLPVRGAAGCTFGGKVY |
| 1b | .......................................................... |
| 1c | .......................................................... |
| 1d | .......................................................... |
| 59a | ALDETWHPDLGEPFGVMRCVLCACEAPQWGRRTRGPGRVSCKNIKPECPTPACGQPRQ |
| 59b | .......................................................... |
| 59c | .......................................................... |
| 59d | .......................................................... |
| 117a | LPGHCCQTCPQERSSSERQPSGLSFEYPRDPEHRSYSDRGEPGAEERARGDGHTDFVA |
| 117b | .......................................................... |
| 117c | .......................................................... |
| 117d | .......................................................... |
| 175a | LLTRSSLRGPRSQAVARARVSLLFSISYRRLDRPTRIRFSDSNGSVLFEHPAAPTQDG |
| 175b | .......................................................... |
| 175c | .......................................................... |
| 175d | .......................................................... |
| 233a | LVCGVWRAVPRLSLRLLRAEQLHVALVTLTHPSGEVWGPLIRHRALAAETFSAILTLE |
| 233b | .......................................................... |
| 233c | .......................................................... |
| 233d | .......................................................... |
| 291a | GPPQQGVGGITLLTLSDTEDSLHFLLLFRGLLEPRSGGLTQVPLRLQILHQGQLLREL |
| 291b | .......................................................... |
| 291c | .......................................................... |
| 291d | .......................................................... |
| 349a | QANVSAQEPGFAEVLPNLTVQEMDWLVLGELQMALEWAGRPGLRISGHIAARKSCDVL |
| 349b | .......................................................... |
| 349c | .......................................................... |
| 349d | .......................................................... |
| 407a | QSVLCGADALIPVQTGAAGSASLTLLGNGSLIYQVQVVGTSSEVVAMTLETKPQRRDQ |
| 407b | .......................................................... |
| 407c | .......................................................... |
| 407d | .......................................................... |
| 465a | RTVLCHMAGLQPGGHTAVGICPGLGARGAHMLLQNELFLNVGTKDFPDGELRGHVAAL |
| 465b | .......................................................... |
| 465c | .......................................................... |
| 465d | .......................................................... |
| 523a | PYCGHSARHDTLPVPLAGALVLPPVKSQAAGHAWLSLDTHCHLHYEVLLAGLGGSEQG |
| 523b | .......................................................... |
| 523c | .......................................................... |
| 523d | .......................................................... |
| 581a | TVTAHLLGPPGTPGPRRLLKGFYGSEAQGVVKDLEPELLRHLAKGMASLLITTKGSPR |
| 581b | .......................................................... |
| 581c | .......................................................... |
| 581d | .......................................................... |
| 639a | GELRGQVHIANQCEVGGLRLEAAGAEGVRALGAPDTASAAPPVVPGLPALAPAKPGGP |
| 639b | .......................................................... |
| 639c | .......................................................... |
| 639d | .......................................................... |
| 697a | GRPRDPNTCFFEGQQRPHGARWAPNYDPLCSLCTCQRRTVICDPVVCPPPSCPHPVQA |
| 697b | .......................................................... |
| 697c | .......................................................... |
| 697d | .......................................................... |
| 755a | PDQCCPVCPEKQDVRDLPGL.....PRS....RDPGEGCYFDGDRSWRAAGTRWHPVV |
| 755b | ......MVPEVRVLSSLLGLALLWFPLDSHARARPDMFCLFHGKR..YSPGESWHPYL |
| 755c | ......MVPEVRVLSSLLGLALLWFPLDSHARARPDMFCLFHGKR..YSPGESWHPYL |
| 755d | ......MVPEVRVLSSLLGLALLWFPLDSHARARPDMFCLFHGKR..YSPGESWHPYL |
| 813a | PPFGLIKCAVCTCKGGTGEVHCEKVQCPRLACAQPVRVNPTDCCKQCPVGSGAHPQLG |
| 813b | EPQGLMYCLRCTCSEG.AHVSCYRLHCPPVHCPQPV.TEPQQCCPKC.VEPHTPSGLR |
| 813c | EPQGLMYCLRCTCSEG.AHVSCYRLHCPPVHCPQPV.TEPQQCCPKC.VEPHTPSGLR |
| 813d | EPQGLMYCLRCTCSEG.AHVSCYRLHCPPVHCPQPV.TEPQQCCPKC.VEPHTPSGLR |
| 871a | DPMQADGRRGCRFAGQWFPESQ..SWHPSVPPFGEMSCITCRCGAGVPHCERDDCSLP |
| 871b | AP.....PKSCQHNGTMYQHGEIFSAHELFPSRLPNQCVLCSCTEGQIYCGLTTCPEP |
| 871c | AP.....PKSCQHNGTMYQHGEIFSAHELFPSRLPNQCVLCSCTMRQVSNRMKRTVCS |
| 871d | AP.....PKSCQHNGTMYQHGEIFSAHELFPSRLPNQCVLCSCTEGQIYCGLTTCPEP |
| 929a | LSCGS..GKESRCCSRCTAHRRPAPETRTDPELEKEAEGS.................. |
| 929b | .GCPAPLPLPDSCCQACKDEASEQSDEEDSVQSLHGVRHPQDPCSSDAGRKRGPGTPA |
| 929c | RSMG...................................................... |
| 929d | .GCPAPLPLPDSCCQACKDEASEQSDEEDSVQSLHGVRHPQDPCSSDAGRKRGPGTPA |
| 987a | .......................................................... |
| 987b | PTGLSAPLSFIPRHFRPKGAGSTTVKIVLKEKHKKACVHGGKTYSHGEVWHPAFRAFG |
| 987c | .......................................................... |
| 987d | PTGLSAPLSFIPRHFRPKGAGSTTVKIVLKEKHKKACVHGGKTYSHGEVWHPAFRAFG |
| 1045a | .......................................................... |
| 1045b | PLPCILCTCEDGRQDCQRVTCPTEYPCRHPEKVAGKCCKICPEDKADPGHSEISSTRC |
| 1045c | .......................................................... |
| 1045d | PLPCILCTCEDGRQDCQRVTCPTEYPCRHPEKVAGKCCKICPEDKADPGHSEISSTRC |
| 1103a | .......................................................... |
| 1103b | PKAPGRVLVHTSVSPSPDNLRRFALEHEASDLVEIYLWKLVKGIFHLTQIKKVRKQDF |
| 1103c | .......................................................... |
| 1103d | PKAPGRVLVHTSVSPSPDNLRRFALEHEASDLVEIYLWKLVKDEETEAQRGEVPGPRP |
| 1161a | .......................................................... |
| 1161b | QKEAQHFRLLAGPHEGHWNVFLAQTLELKVTASPDKVTKT.................. |
| 1161c | .......................................................... |
| 1161d | HSQNLPLDSDQESQEARLPERGTALPTARWPPRRSLERLPSPDPGAEGHGQSRQSDQD |
| 1219a | .... |
| 1219b | .... |
| 1219c | .... |
| 1219d | ITKT |

TABLE 8
Comparison of cysteine-rich repeat motifs.

# amino acids between cysteine residues

| cysteine | human | | | |
|---|---|---|---|---|
| residues | chordin | LP226 | LP233 | LP236 |
| C1-C2 | 24 | 24-26 | 24 | 24-26 |
| C2-C3 | 2 | 2 | 2 | 2 |
| C3-C4 | 1 | 1-2 | 1 | 1 |
| C4-C5 | 6-8 | 6-7 | 7 | 6-7 |
| C5-C6 | 4 | 4 | 4 | 4 |
| C6-C7 | 4 | 4-5 | 4 | 4-5 |
| C7-C8 | 12 | 9 | 9 | 9 |
| C8-C9 | 0 | 0 | 0 | 0 |
| C9-C10 | 2 | 2 | 2 | 2 |
| C10-R/K | 3 | 4-/ | / | 3-/ |

The term “LP,” when used herein, encompasses native sequence LP226, LP233, or LP236 polynucleotide, polypeptide or polypeptide variants thereof (which are further defined herein). The LP226, LP233, or LP236 polypeptides may be isolated from a variety of sources, such as from human tissue types or from another source, or prepared by recombinant or synthetic methods.

A “native sequence LP polypeptide” comprises a polypeptide having the same amino acid sequence as an LP226, LP233, or LP236 polypeptide derived from nature. Such native sequence LP226, LP233, or LP236 polypeptide can be isolated from nature or can be produced by recombinant or synthetic means. The term “native sequence LP polypeptide” specifically encompasses naturally-occurring truncated or secreted forms of an LP226, LP233, or LP236 polypeptide, (e.g., soluble forms containing an extracellular domain sequence), naturally-occurring variant forms (e.g., alternatively spliced forms) and naturally-occurring allelic variants of an LP226, LP233, or LP236 polypeptide.

In one embodiment of the invention, the native sequence LP polypeptide is a full-length or mature native sequence LP226, LP233, or LP236 polypeptide comprising amino acids 1 or 27 through 430 of SEQ ID NO:2; 1 or 27 through 162 of SEQ ID NO:4; and 1 or 27 through 451 of SEQ ID NO:6, respectively. Also, while the LP polypeptides disclosed are shown to begin with a methionine residue designated as amino acid position 1, it is conceivable and possible that another methionine residue located either upstream or downstream from amino acid position 1 may be employed as the starting amino acid residue.

In preferred embodiments, a “portion” of an LP polypeptide sequence is at least about 20, 25, 30, 35, 40, 45, 50, 55, 60, 65, 70, 75, 80, 85, 90, 95, or 100 contiguous amino acid residues in length.

“LP variant” means an “active” LP226, LP233, or LP236 polypeptide as defined below, having at least about 90% amino acid sequence identity with the LP polypeptide, having the deduced amino acid sequences shown above. Such LP polypeptide variants include, for instance, LP226, LP233, or LP236, wherein one or more amino acid residues are added, substituted or deleted, at the N- or C-terminus or within the sequences shown. Ordinarily, an LP polypeptide variant will have at least about 90% amino acid sequence identity, preferably at least about 91% sequence identity, yet more preferably at least about 92% sequence identity, yet more preferably at least about 93% sequence identity, yet more preferably at least about 94% sequence identity, yet more preferably at least about 95% sequence identity, yet more preferably at least about 96% sequence identity, yet more preferably at least about 97% sequence identity, yet more preferably at least about 98% sequence identity, yet more preferably at least about 99% amino acid sequence identity with the amino acid sequence described, with or without the signal peptide.

“Percent (%) amino acid sequence identity” with respect to the LP amino acid sequences identified herein is defined as the percentage of amino acid residues in a candidate sequence that are identical with the amino acid residues in an LP polypeptide sequence, after aligning the sequences and introducing gaps, if necessary, to achieve the maximum percent sequence identity, and not considering any conservative amino acid substitutions as part of the sequence identity. Alignment for purposes of determining percent amino acid sequence identity can be achieved in various ways that are within the skill in the art, for instance, using publicly available computer software such as ALIGN, ALIGN-2, Megalign (DNASTAR) or BLAST (e.g., Blast, Blast-2, WU-Blast-2) software. Those skilled in the art can determine appropriate parameters for measuring alignment, including any algorithms needed to achieve maximal alignment over the full length of the sequences being compared. The percent identity values used herein are generated using WU-BLAST-2 [Altschul, et al., Methods in Enzymology 266: 460-480 (1996)]. Most of the WU-BLAST-2 search parameters are set to the default values. Those not set to default values, i.e., the adjustable parameters, are set with the following values: overlap span=1; overlap fraction=0.125; word threshold (T)=11; and scoring matrix=BLOSUM 62. For purposes herein, a percent amino acid sequence identity value is determined by dividing (a) the number of matching identical amino acid residues between the amino acid sequence of the LP polypeptide of interest and the comparison amino acid sequence of interest (i.e., the sequence against which the LP polypeptide of interest is being compared) as determined by WU-BLAST-2, by (b) the total number of amino acid residues of the LP polypeptide of interest, respectively.

An “LP variant polynucleotide” or “LP variant nucleic acid sequence” means an active LP polypeptide-encoding nucleic acid molecule as defined below having at least about 75% nucleic acid sequence identity with SEQ ID NO:1, 3, or 5. Ordinarily, an LP polypeptide will have at least about 75% nucleic acid sequence identity, more preferably at least about 80% nucleic acid sequence identity, yet more preferably at least about 81% nucleic acid sequence identity, yet more preferably at least about 82% nucleic acid sequence identity, yet more preferably at least about 83% nucleic acid sequence identity, yet more preferably at least about 84% nucleic acid sequence identity, yet more preferably at least about 85% nucleic acid sequence identity, yet more preferably at least about 86% nucleic acid sequence identity, yet more preferably at least about 87% nucleic acid sequence identity, yet more preferably at least about 88% nucleic acid sequence identity, yet more preferably at least about 89% nucleic acid sequence identity, yet more preferably at least about 90% nucleic acid sequence identity, yet more preferably at least about 91% nucleic acid sequence identity, yet more preferably at least about 92% nucleic acid sequence identity, yet more preferably at least about 93% nucleic acid sequence identity, yet more preferably at least about 94% nucleic acid sequence identity, yet more preferably at least about 95% nucleic acid sequence identity, yet more preferably at least about 96% nucleic acid sequence identity, yet more preferably at least about 97% nucleic acid sequence identity, yet more preferably at least about 98% nucleic acid sequence identity, yet more preferably at least about 99% nucleic acid sequence identity with the nucleic acid sequences shown in SEQ ID NO:1, 3, or 5. Variants specifically exclude or do not encompass the native nucleotide sequence, as well as those prior art sequences that share 100% identity with the nucleotide sequences of the invention.

“Percent (%) nucleic acid sequence identity” with respect to the LP polynucleotide sequences identified herein is defined as the percentage of nucleotides in a candidate sequence that are identical with the nucleotides in the LP polynucleotide sequence after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as ALIGN, Align-2, Megalign (DNASTAR), or BLAST (e.g., Blast, Blast-2) software. Those skilled in the art can determine appropriate parameters for measuring alignment, including any algorithms needed to achieve maximal alignment over the full length of the sequences being compared. For purposes herein, however, percent nucleic acid identity values are generated using the WU-BLAST-2 (BlastN module) program (Altschul, et al., Methods in Enzymology 266: 460-480 (1996)). Most of the WU-BLAST-2 search parameters are set to the default values. Those not set default values, i.e., the adjustable parameters, are set with the following values: overlap span=1; overlap fraction=0.125; word threshold (T)=11; and scoring matrix=BLOSUM62. For purposes herein, a percent nucleic acid sequence identity value is determined by dividing (a) the number of matching identical nucleotides between the nucleic acid sequence of the LP polypeptide-encoding nucleic acid molecule of interest and the comparison nucleic acid molecule of interest (i.e., the sequence against which the LP polypeptide-encoding nucleic acid molecule of interest is being compared) as determined by WU-BLAST-2, by (b) the total number of nucleotides of the LP polypeptide-encoding nucleic acid molecule of interest.

In other embodiments, the LP variant polypeptides are encoded by nucleic acid molecules which are capable of hybridizing, preferably under stringent hybridization and wash conditions, to nucleotide sequences encoding the full-length LP polypeptide shown in SEQ ID NO:2, 4, and 6. This scope of variant polynucleotides specifically excludes those sequences that are known as of the filing and/or priority dates of the present application.

The term “mature protein” or “mature polypeptide” as used herein refers to the form(s) of the protein produced by expression in a mammalian cell. It is generally hypothesized that once export of a growing protein chain across the rough endoplasmic reticulum has been initiated, proteins secreted by mammalian cells have a signal peptide (SP) sequence which is cleaved from the complete polypeptide to produce a “mature” form of the protein. Oftentimes, cleavage of a secreted protein is not uniform and may result in more than one species of mature protein. The cleavage site of a secreted protein is determined by the primary amino acid sequence of the complete protein and generally cannot be predicted with complete accuracy. Methods for predicting whether a protein has an SP sequence, as well as the cleavage point for that sequence, are available. A cleavage point may exist within the N-terminal domain between amino acid 10 and amino acid 35. More specifically the cleavage point is likely to exist after amino acid 15 but before amino acid 30, more likely after amino acid 27. As one of ordinary skill would appreciate, however, cleavage sites sometimes vary from organism to organism and cannot be predicted with absolute certainty. Optimally, cleavage sites for a secreted protein are determined experimentally by amino-terminal sequencing of the one or more species of mature proteins found within a purified preparation of the protein.

The term “positives”, in the context of sequence comparison performed as described above, includes residues in the sequences compared that are not identical but have similar properties (e.g., as a result of conservative substitutions). The percent identity value of positives is determined by the fraction of residues scoring a positive value in the BLOSUM 62 matrix. This value is determined by dividing (a) the number of amino acid residues scoring a positive value in the BLOSUM62 matrix of WU-BLAST-2 between the LP polypeptide amino acid sequence of interest and the comparison amino acid sequence (i.e., the amino acid sequence against which the LP polypeptide sequence is being compared) as determined by WU-BLAST-2, by (b) the total number of amino acid residues of the LP polypeptide of interest.

“Isolated,” when used to describe the various polypeptides disclosed herein, means a polypeptide that has been identified and separated and/or recovered from a component of its natural environment. Preferably, the isolated polypeptide is free of association with all components with which it is naturally associated. Contaminant components of its natural environment are materials that would typically interfere with diagnostic, prophylactic, or therapeutic uses for the polypeptide, and may include enzymes, hormones, and other proteinaceous or non-proteinaceous solutes. In preferred embodiments, the polypeptide will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated polypeptide includes polypeptide in situ within recombinant cells, since at least one component of the LP polypeptide natural environment will not be present. Ordinarily, however, isolated polypeptide will be prepared by at least one purification step.

An “isolated” LP polypeptide-encoding nucleic acid molecule is a nucleic acid molecule that is identified and separated from at least one contaminant nucleic acid molecule with which it is ordinarily associated in the natural source of the LP polypeptide-encoding nucleic acid. An isolated LP polypeptide-encoding nucleic acid molecule is other than in the form or setting in which it is found in nature. Isolated LP polypeptide-encoding nucleic acid molecules, therefore, are distinguished from the LP polypeptide-encoding nucleic acid molecule as it exists in natural cells. However, an isolated LP polypeptide-encoding nucleic acid molecule includes LP polypeptide-encoding nucleic acid molecules contained in cells that ordinarily express LP polypeptide where, for example, the nucleic acid molecule is in a chromosomal location different from that of natural cells.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “amino acid” is used herein in its broadest sense, and includes naturally occurring amino acids as well as non-naturally occurring amino acids, including amino acid analogs and derivatives. The latter includes molecules containing an amino acid moiety. One skilled in the art will recognize, in view of this broad definition, that reference herein to an amino acid includes, for example, naturally occurring proteogenic L-amino acids; D-amino acids; chemically modified amino acids, such as amino acid analogs and derivatives; naturally-occurring non-proteogenic amino acids such as norleucine, beta-alanine, ornithine, etc.; and chemically synthesized compounds having properties known in the art to be characteristic of amino acids. As used herein, the term “proteogenic” indicates that the amino acid can be incorporated into a peptide, polypeptide, or protein in a cell through a metabolic pathway.

“Stringency” of hybridization reactions is readily determinable by one of ordinary skill in the art, and generally is an empirical calculation dependent upon probe length, washing temperature, and salt concentration. In general, longer nucleic acid probes required higher temperatures for proper annealing, while shorter nucleic acid probes need lower temperatures. Hybridization generally depends on the ability of denatured DNA to reanneal when complementary strands are present in an environment below their melting temperature. The higher the degree of desired homology between the probe and hybridizable sequence, the higher the relative temperature that can be used. As a result, it follows that higher relative temperatures would tend to make the reactions more stringent, while lower temperatures less so. For additional details and explanation of stringency of hybridization reactions, see Ausubel, et al., Current Protocols in Molecular Biology, Wiley Interscience Publishers, 1995.

“Stringent conditions” or “high stringency conditions”, as defined herein, may be identified by those that (1) employ low ionic strength and high temperature for washing, for example, 15 mM sodium chloride/1.5 mm sodium citrate/0.1% sodium dodecyl sulfate at 50 degrees C.; (2) employ during hybridization a denaturing agent, such as formamide, for example, 50% (v/v) formamide with 0.1% bovine serum albumin/0.1% ficoll/0.1% polyvinylpyrrolidone/50 mM sodium phosphate buffer at pH 6.5 with 750 mM sodium chloride/75 mM sodium citrate at 42 degrees C.; or (3) employ 50% formamide, 5×SSC (750 mM sodium chloride, 75 mM sodium citrate), 50 mM sodium phosphate (pH 6.8), 0.1% sodium pyrophosphate, 5× Denhardt's solution, sonicated salmon sperm DNA (50 μg/mL), 0.1% SDS, and 10% dextran sulfate at 42 degrees C. with washes at 42 degrees C. in 0.2×SSC (30 mM sodium chloride/3 mM sodium citrate) and 50% formamide at 55 degrees C., followed by a high-stringency wash consisting of 0.1×SSC containing EDTA at 55 degrees C.

“Moderately stringent conditions” may be identified as described by Sambrook, et al. [Molecular Cloning: A Laboratory Manual, New York: Cold Spring Harbor Press, (1989)], and include the use of washing solution and hybridization conditions (e.g., temperature, ionic strength and %SDS) less stringent than those described above. An example of moderately stringent conditions is overnight incubation at 37 degrees C. in a solution comprising: 20% formamide, 5×SSC (750 mM sodium chloride, 75 mM sodium citrate), 50 mM sodium phosphate at pH 7.6, 5× Denhardt's solution, 10% dextran sulfate, and 20 mg/mL denatured sheared salmon sperm DNA, followed by washing the filters in 1×SSC at about 37 to 50 degrees C. The skilled artisan will recognize how to adjust the temperature, ionic strength, etc., as necessary to accommodate factors such as probe length and the like.

The term “epitope tagged” where used herein refers to a chimeric polypeptide comprising an LP polypeptide, or domain sequence thereof, fused to a “tag polypeptide.” The tag polypeptide has enough residues to provide an epitope against which an antibody may be made, or which can be identified by some other agent, yet is short enough such that it does not interfere with the activity of the LP polypeptide. The tag polypeptide preferably is also fairly unique so that the antibody does not substantially cross-react with other epitopes. Suitable tag polypeptides generally have at least six amino acid residues and usually between about eight to about fifty amino acid residues (preferably, between about ten to about twenty residues).

As used herein, the term “immunoadhesin,” sometimes referred to as an Fc fusion, designates antibody-like molecules that combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG-1, IgG-2, IgG-3 or IgG-4 subtypes, IgA (including IgA-1 and IgA-2), IgE, IgD or IgM.

“Active” or “activity” for the purposes herein refers to form(s) of LP polypeptide which retain all or a portion of the biologic and/or immunologic activities of a native or naturally-occurring LP polypeptide. Elaborating further, “biological” activity refers to a biological function (either inhibitory or stimulatory) caused by a native or naturally-occurring LP polypeptide other than the ability to induce the production of an antibody against an antigenic epitope possessed by a native or naturally-occurring LP polypeptide. An “immunological” activity refers only to the ability to induce the production of an antibody against an antigenic epitope possessed by a native or naturally-occurring LP polypeptide. A preferred biological activity of LP226, LP233, or LP236 includes, for example, the ability to treat osteosarcoma, prostate cancer, breast cancer, renal carcinoma, thyroid carcinoma, multiple myeloma, lung cancer, salivary gland mucoepidermoid carcinoma, chronic lymphocytic leukemia, T cell lymphoblastic leukemia, cardiovascular disease, thrombosis, heart failure, Alzheimer's disease, Parkinson's disease, Huntington's disease, schizophrenia, multiple sclerosis, tuberous sclerosis, prepubertal periodontitis, Papillon-Lefevre syndrome, kidney fibrosis, cirrhosis, idiopathic lung fibrosis, chronic asbestosis, pneumonitis, acute respiratory distress syndrome (ARDS), cardiac fibrosis, atherosclerosis, restenosis, macular degeneration, retinal and vitreal retinopathy, keloids, Crohn's disease, and valvular ossification.

A preferred biological activity for an antagonist of LP226, LP233, or LP236 includes wound and tissue repair, such as healing of chronic ulcers, burns, and fractured or alternatively broken skin, bone, nerve, or other tissue.

Another preferred biological activity for an antagonist of LP226, LP233, or LP236 includes, for example, the ability to treat conditions resulting in loss of bone mineral content such as achondroplasia, cleidocranial dysostosis, enchondromatosis, fibrous dysplasia, Gaucher's disease, hypophosphatemic rickets, Marfan's disease, multiple hereditary exotoses, neurofibromatosis, osteogenesis imperfecta, osteopetrosis, osteopoikilosis, sclerotic lesions, fractures, pseudoartritis, and pyogenic osteomyelitis.

Yet another preferred biological activity for an antagonist of LP226, LP233, or LP236 includes the ability to treat conditions which cause osteopenia such as anemia, steroid usage, conditions caused by heparin, bone marrow disorders, scurvy, malnutrition, calcium deficiency, idiopathic osteoporosis, congenital osteopenia or osteoporosis, chronic liver disease, oligomenorrhea, amenorrhea, diabetes mellitus, hyperthyroidism, Cushing's disease, acromegaly, hypogonadism, reflex sympathetic dystrophy syndrome, transient regional osteoporosis, and osteomalacia.

The term “antagonist” is used in the broadest sense and includes any molecule that partially or fully blocks, inhibits, or neutralizes a biological activity of a native LP polypeptide disclosed herein. In a similar manner, the term “agonist” is used in the broadest sense and includes any molecule that mimics a biological activity of a native LP polypeptide disclosed herein. Suitable agonist or antagonist molecules specifically include agonist or antagonist antibodies or antibody fragments, fragments or amino acid sequence variants of native LP polypeptides, peptides, small organic molecules, etc. Methods for identifying agonists or antagonists of an LP polypeptide may comprise contacting an LP polypeptide with a candidate agonist or antagonist molecule and measuring a detectable change in one or more biological activities normally associated with the LP polypeptide.

“BMP” and “BMPs” refer to the family of bone morphogenetic proteins, including such members of the family as BMP-4, which LP226 was found to bind antagonistically.

“TGFSβ,” “TGF-β,” “TGF-b,” “TGF-beta,” and “TGF-betas” refer to the superfamily of transforming growth factors. These include, but are not limited to, TGF-b1, TGF-b1.2, TGF-b2, which exhibited strong binding to LP226, and the BMP and GDF (growth differential factors) families.

“Antibodies” (Abs) and “immunoglobulins” (Igs) are glycoproteins having the same structural characteristics. While antibodies exhibit binding specificity to a specific antigen, immunoglobulins include both antibodies and other antibody-like molecules that lack antigen specificity. Polypeptides of the latter kind are, for example, produced at low levels by the lymph system and at increased levels by myelomas. The term “antibody” is used in the broadest sense and specifically covers, without limitation, intact monoclonal antibodies, polyclonal antibodies, multispecific antibodies (e.g., bispecific antibodies) formed from at least two intact antibodies, and antibody fragments so long as they exhibit the desired biological activity.

The terms “treating”, “treatment,” and “therapy” as used herein refer to curative therapy, prophylactic therapy, and preventive therapy. An example of “preventive therapy” is the prevention or lessened targeted pathological condition or disorder. Those in need of treatment include those already with the disorder as well as those prone to have the disorder or those in whom the disorder is to be prevented.

“Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption but, rather, is cyclic in nature.

Administration “in combination with” one or more further therapeutic agents includes simultaneous (concurrent) and consecutive administration in any order.

A “therapeutically-effective amount” is the minimal amount of active agent (e.g., an LP polypeptide, antagonist or agonist thereof) which is necessary to impart therapeutic benefit to a mammal. For example, a “therapeutically-effective amount” to a mammal suffering or prone to suffering or to prevent it from suffering from an osteosarcoma, prostate cancer, breast cancer, renal carcinoma, thyroid carcinoma, multiple myeloma, lung cancer, salivary gland mucoepidermoid carcinoma, chronic lymphocytic leukemia, T cell lymphoblastic leukemia, schizophrenia, tuberous sclerosis, prepubertal periodontitis, Papillon-Lefevre syndrome, achondroplasia, cleidocranial dysostosis, enchondromatosis, fibrous dysplasia, Gaucher's disease, hypophosphatemic rickets, marfan's disease, multiple hereditary exotoses, neurofibromatosis, osteogenesis imperfecta, osteopetrosis, osteopoikilosis, sclerotic lesions, fractures, pseudoartritis, pyogenic osteomyelitis, anemia, steroid usage, conditions caused by heparin, bone marrow disorders, scurvy, malnutrition, calcium deficiency, idiopathic osteoporosis, congenital osteopenia or osteoporosis, chronic liver disease, oligomenorrhea, amenorrhea, diabetes mellitus, Cushing's disease, hyperthyroidism, acromegaly, hypogonadism, reflex sympathetic dystrophy syndrome, transient regional osteoporosis, osteomalacia, valvular ossification, cardiovascular disease, chronic wound, diabetic ulcer, pressure ulcer, venous stasis ulcer, burn, incision, bone fracture, torn cartilage, connective tissue, tooth, spinal cord, nerve or other tissue-defective disorder is such an amount which induces, ameliorates or otherwise causes an improvement in the pathological symptoms, disease progression, physiological conditions associated with or resistance to succumbing to the aforedescribed disorder.

A “neurological disorder” is a disorder principally characterized by a typical function of the nervous system. Neurological disorders include, but are not limited to, Alzheimer's disease, Parkinson's disease, Huntington's disease, schizophrenia, and multiple sclerosis.

A “cardiovascular disorder” is a disorder principally characterized by a typical function of the heart or blood vessels. Cardiovascular disorders include, but are not limited to, atherosclerosis, thrombosis, valvular ossification, and heart failure.

A “proliferative disorder” is a disorder principally characterized by abnormal proliferation of cells. Proliferative disorders include, but are not limited to, osteosarcoma, prostate cancer, breast cancer, renal carcinoma, thyroid carcinoma, multiple myeloma, lung cancer, salivary gland mucoepidermoid carcinoma, chronic lymphocytic leukemia, and T cell lymphoblastic leukemia.

A “disorder requiring wound and tissue repair” is a disorder principally characterized by abnormal or slow tissue healing. Wound and tissue disorders include, but are not limited to, bone, cartilage, tendon, tooth, spinal cord, nerve, or skin disorders such as fractures, burns, ulcers, or chronic wounds.

A “fibrotic disorder” refers to a disorder principally characterized by excessive production and deposition of collagen, yielding excessive scarring. Fibrotic disorders include, but are not limited to, kidney fibrosis, cirrhosis, idiopathic lung fibrosis, chronic asbestosis, pneumonitis, acute respiratory distress syndrome (ARDS), cardiac fibrosis, atherosclerosis, restenosis, macular degeneration, retinal and vitreal retinopathy, keloids, and Crohn's disease.

“Carriers” as used herein include pharmaceutically-acceptable carriers, excipients, or stabilizers which are nontoxic to the cell or mammal being exposed thereto at the dosages and concentrations employed. Often the physiologically-acceptable carrier is an aqueous pH buffered solution. Examples of physiologically acceptable carriers include buffers such as phosphate, citrate, and other organic acids; antioxidants including ascorbic acid; low molecule weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, polyethylene glycol (PEG), and PLURONIC®.

“Antibody fragments” comprise a portion of an intact antibody, preferably the antigen binding or variable region of the intact antibody. Examples of antibody fragments include Fab, Fab′, F(ab′)₂ and Fv fragments; diabodies; linear antibodies [Zapata, et al., Protein Engin. 8 (10): 1057-62 (1995)]; single-chain antibody molecules; and multispecific antibodies formed from antibody fragments.

“Fv” is the minimum antibody fragment which contains a complete antigen-recognition and binding site. This region consists of a dimer of one heavy- and one light-chain variable domain in tight, non-covalent association. It is in this configuration that the three CDRs of each variable domain interact to define an antigen-binding site on the surface of the VHVL dimer. Collectively, the six CDRs confer antigen-binding specificity to the antibody. However, even a single variable domain (or half of an Fv comprising only three CDR specific for an antigen) has the ability to recognize and bind antigen, although at a lower affinity than the entire binding site.

“Single-chain Fv” or “sFv” antibody fragments comprise the VHand V_{L} domains of antibody, wherein these domains are present in a single polypeptide chain. Preferably, the Fv polypeptide further comprises a polypeptide linker between the V_{H} and V_{L} domain, which enables the sFv to form the desired structure for antigen binding. For a review of sFv, see Pluckthun, The Pharmacology of Monoclonal Antibodies, vol. 113, Rosenburg and Moore, eds., Springer-Verlag, New York, pp. 269-315 (1994).

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404.097, WO 93/11161; and Hollinger, et al., Proc. Natl. Acad. Sci. USA 90: 6444-8 (1993).

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or non-proteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue, or preferably, silver stain. Isolated antibody includes the antibody in situ within recombinant cells since at least one component of the antibody's natural environment will not be present. Ordinarily, however, isolated antibody will be prepared by at least one purification step.

A “liposome” is a small vesicle composed of various types of lipids, phospholipids and/or surfactant which is useful for delivery of a drug (such as an LP polypeptide or antibody thereto) to a mammal. The components of the liposome are commonly arranged in a bilayer formation, similar to the lipid arrangement of biological membranes.

A “small molecule” is defined herein to have a molecular weight below about 500 daltons.

The term “modulate” means to affect (e.g., either upregulate, downregulate or otherwise control) the level of a signaling pathway. Cellular processes under the control of signal transduction include, but are not limited to, transcription of specific genes, normal cellular functions, such as metabolism, proliferation, differentiation, adhesion, apoptosis and survival, as well as abnormal processes, such as transformation, blocking of differentiation and metastasis.

The LP polynucleotide can be composed of any polyribonucleotide or polydeoxyribonucleotide, which may be unmodified RNA or DNA or modified RNA or DNA. For example, the LP polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, the LP polynucleotides can be composed of triple-stranded regions comprising RNA or DNA or both RNA and DNA. LP polynucleotides may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

LP polypeptides can be composed of amino acids joined to each other by peptide bonds or modified peptide bonds, i.e., peptide isosteres, and may contain amino acids other than the gene-encoded amino acids. The LP polypeptides may be modified by either natural processes, such as post-translational processing, or by chemical modification techniques which are well known in the art. Such modifications are well described in basic texts and in more detailed monographs, as well as in a voluminous research literature. Modifications can occur anywhere in the LP polypeptides, including the peptide backbone, the amino acid side-chains and the amino or carboxyl termini. It will be appreciated that the same type of modification may be present in the same or varying degrees at several sites in a given LP polypeptide. Also, a given LP polypeptide may contain many types of modifications. LP polypeptides may be branched, for example, as a result of ubiquitination, and they may be cyclic, with or without branching. Cyclic, branched, and branched cyclic LP polypeptides may result from post-translation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. See, for instance, Creighton, Proteins—Structure and Molecular Properties, 2nd Ed., W. H. Freeman and Company, New York (1993); Johnson, Post-transational Covalent Modification of Proteins, Academic Press, New York, pp. 1-12 (1983); Seifter, et al. Meth. Enzymol. 182: 626-46 (1990); Rattan, et al., Ann. NY Acad. Sci. 663: 48-62 (1992).

Variations in the native full-length sequence LP polypeptide or in various domains of the LP polypeptide described herein can be made, for example, using any of the techniques and guidelines for conservative and non-conservative mutations set forth, for instance, in U.S. Pat. No. 5,364,934. Variations may be a substitution, deletion or insertion of one or more codons encoding LP polypeptide that results in a change in the amino acid sequence of the LP polypeptide as compared with the native sequence LP polypeptide. Optionally the variation is by substitution of at least one amino acid with any other amino acid in one or more of the domains of the LP polypeptide. Guidance in determining which amino acid residue may be inserted, substituted or deleted without adversely affecting the desired activity may be found by comparing the sequence of the LP polypeptide with that of homologous known protein molecules and minimizing the number of amino acid sequence changes made in regions of high homology. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of 1 to 5 amino acids. The variation allowed may be determined by systematically making insertions, deletions or substitutions of amino acids in the sequence and testing the resulting variants for activity (such as in any of the in vitro assays described herein) for activity exhibited by the full-length or mature native sequence.

LP polypeptide fragments are provided herein. Such fragments may be truncated at the N-terminus or C-terminus, or may lack internal residues, for example, when compared with a full length or native protein. Certain fragments lack amino acid residues that are not essential for a desired biological activity of the LP polypeptide.

LP polypeptide fragments may be prepared by any of a number of conventional techniques. Desired peptide fragments may be chemically synthesized. An alternative approach involves generating LP polypeptide fragments by enzymatic digestion, e.g., by treating the protein with an enzyme known to cleave proteins at sites defined by particular amino acid residues, or by digesting the DNA with suitable restriction enzymes and isolating the desired fragment. Yet another suitable technique involves isolating and amplifying a DNA fragment encoding a desired polypeptide fragment by polymerase chain reaction (PCR). Oligonucleotides that define the desired termini of the DNA fragment are employed at the 5′ and 3′ primers in the PCR. Preferably, LP polypeptide fragments share at least one biological and/or immunological activity with the native LP polypeptide shown in SEQ ID NO:2, 4, 6.

Covalent modifications of LP polypeptides are included within the scope of this invention. One type of covalent modification includes reacting targeted amino acid residues of an LP polypeptide with an organic derivatizing agent that is capable of reacting with selected side chains or the N- or C-terminal residues of an LP polypeptide. Derivatization with bifunctional agents is useful, for instance, for crosslinking LP polypeptide to a water-insoluble support matrix or surface for use in the method for purifying anti-LP antibodies, and vice-versa. Commonly used crosslinking agents include, e.g., 1,1-bis-(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis-(succinimidylpropionate), bifunctional maleimides such as bis-N-maleimido-1,8-octane and agents such as methyl-3-[(p-azidophenyl)-dithiolpropioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the alpha-amino groups of lysine, arginine, and histidine side chains [T. E. Creighton. Proteins: Structure and Molecular Properties, W. H. Freeman & Co., San Francisco, pp. 79-86 (1983)], acetylation of the N-terminal amine, and amidation of any C-terminal carboxyl group.

Another type of covalent modification of the LP polypeptides included within the scope of this invention comprises altering the native glycosylation pattern of the polypeptide. “Altering the native glycosylation pattern” is intended for purposes herein to mean deleting one or more carbohydrate moieties found in native sequence LP polypeptide and/or adding one or more glycosylation sites that are not present in the native sequences of LP polypeptides. Additionally, the phrase includes qualitative changes in the glycosylation of the native proteins, involving a change in the nature and proportions of the various carbohydrate moieties present.

Addition of glycosylation sites to LP polypeptides may be accomplished by altering the amino acid sequence thereof. The alteration may be made, for example, by the addition of, or substitution by, one or more serine or threonine residues to the native sequences of LP polypeptides (for O-linked glycosylation sites). The LP amino acid sequences may optionally be altered through changes at the DNA level, particularly by mutating the DNA encoding the LP polypeptides at preselected bases such that codons are generated that will translate into the desired amino acids.

Another means of increasing the number of carbohydrate moieties on the LP polypeptides is by chemical or enzymatic coupling of glycosides to the polypeptide. Such methods are described in the art, e.g., in WO 87/05330, published Sep. 11, 1987, and in Aplin and Wriston, CRC Crit. Rev. Biochem., pp. 259-306 (1981).

Removal of carbohydrate moieties present on the LP polypeptide may be accomplished chemically or enzymatically or by mutational substitution of codons encoding for amino acid residues that serve as targets for glycosylation. Chemical deglycosylation techniques are known in the art and described, for instance, by Sojar, et al., Arch. Biochem. Biophys. 259: 52-7 (1987) and by Edge, et al., Anal. Biochem. 118: 131-7 (1981). Enzymatic cleavage of carbohydrate moieties on polypeptides can be achieved by the use of a variety of endo- and exo-glycosidases as described by Thotakura, et al., Meth. Enzymol. 138: 350-9 (1987).

Another type of covalent modification of LP polypeptides comprises linking any one of the LP polypeptides to one of a variety of nonproteinaceous 20 polymers, e.g., polyethylene glycol, polypropylene glycol, or polyoxyalkylenes, in the manner set forth in U.S. Pat. No. 4,640,835; 4,496,689; 4,301,144; 4,670,417; 4,791,192 or 4,179,337.

LP polypeptides of the present invention may also be modified in a way to form chimeric molecules comprising an LP226, LP233, or LP236 polypeptide fused to another heterologous polypeptide or amino acid sequence. In one embodiment, such a chimeric molecule comprises a fusion of an LP226, LP233, or LP236 polypeptide with a tag polypeptide which provides an epitope to which an anti-tag antibody can selectively bind. The epitope tag is generally placed at the amino- or carboxyl-terminus of LP226, LP233, or LP236 polypeptide. The presence of such epitope-tagged forms of LP polypeptides can be detected using an antibody against the tag polypeptide. Also, provision of the epitope tag enables an LP polypeptide to be readily purified by affinity purification using an anti-tag antibody or another type of affinity matrix that binds to the epitope tag.

In an alternative embodiment, the chimeric molecule may comprise a fusion of an LP polypeptide with an immunoglobulin or a particular region of an immunoglobulin. For a bivalent form of the chimeric molecule, such a fusion could be to the Fc region of an IgG molecule. The Ig fusions preferably include the substitution of a soluble transmembrane domain deleted or inactivated form of an LP polypeptide in place of at least one variable region within an Ig molecule. In a particularly preferred embodiment, the immunoglobulin fusion includes the hinge, CH2 and CH3 or the hinge, CH1, CH2 and CH3 regions of an IgG1 molecule. For the production of immunoglobulin fusions, see also U.S. Pat. No. 5,428,130.

In yet a further embodiment, the LP polypeptides of the present invention may also be modified in a way to form a chimeric molecule comprising an LP polypeptide fused to a leucine zipper. Various leucine zipper polypeptides have been described in the art. See, e.g., Landschulz, et al., Science 240(486G): 1759-64 (1988); WO 94/10308; Hoppe, et al., FEBS Letters 344(2-3): 191-5 (1994); Abel, et al., Nature 341(6237): 24-5 (1989). It is believed that use of a leucine zipper fused to an LP polypeptide may be desirable to assist in dimerizing or trimerizing soluble LP polypeptide in solution. Those skilled in the art will appreciate that zipper may be fused at either the N- or C-terminal end of LP polypeptide.

The description below relates primarily to production of LP polypeptide by culturing cells transformed or transfected with a vector containing LP226, LP233, or LP236 polypeptide encoding nucleic acid. It is, of course, contemplated that alternative methods, which are well known in the art, may be employed to prepare LP polypeptides. For instance, the LP226, LP233, or LP236 sequence, or portions thereof, may be produced by direct peptide synthesis using solid-phase techniques [see, e.g., Stewart, et al., Solid-Phase Peptide Synthesis, W. H. Freeman Co., San Francisco, Calif. (1969); Merrifield, J. Am. Chem. Soc. 85: 2149-54 (1963)]. In vitro protein synthesis may be performed using manual techniques or by automation. Automated synthesis may be accomplished, for instance, using an Applied Biosystems Peptide Synthesizer (Foster City, Calif.) using manufacturer's instructions. Various portions of LP polypeptide may be chemically synthesized separately and combined using chemical or enzymatic methods to produce a full-length LP polypeptide.

DNA encoding an LP polypeptide may be obtained from a cDNA library prepared from tissue believed to possess the LP mRNA and to express it at a detectable level. Libraries can be screened with probes (such as antibodies to an LP polynucleotide or oligonucleotides of at least about 20-80 bases) designed to identify the gene of interest or the protein encoded by it. Screening the cDNA or genomic library with the selected probe may be conducted using standard procedures, such as described in Sambrook, et al., Molecular Cloning: A Laboratory Manual, Cold Spring Harbor Laboratory Press, NY (1989). An alternative means to isolate the gene encoding LP226, LP233, or LP236 is to use PCR methodology [Sambrook, et al., supra; Dieffenbach, et al., PCR Primer: A Laboratory Manual, Cold Spring Harbor Laboratory Press, NY (1995)].

Nucleic acids having protein coding sequence may be obtained by screening selected cDNA or genomic libraries using the deduced amino acid sequence disclosed herein for the first time and, if necessary, using conventional primer extension procedures as described in Sambrook, et al., supra, to detect precursors and processing intermediates of mRNA that may not have been reverse-transcribed into cDNA.

Host cells are transfected or transformed with expression or cloning vectors described herein for LP polypeptide production and cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: A Practical Approach, M. Butler, ed. (IRL Press, 1991) and Sambrook, et al., supra. Methods of transfection are known to the ordinarily skilled artisan, for example, CaPO₄ and electroporation. General aspects of mammalian cell host system transformations have been described in U.S. Pat. No. 4,399,216. Transformations into yeast are typically carried out according to the method of van Solingen, et al., J Bact. 130(2): 946-7 (1977) and Hsiao, et al., Proc. Natl. Acad. Sci. USA 76(8): 3829-33 (1979). However, other methods for introducing DNA into cells, such as by nuclear microinjection, electroporation, bacterial protoplast fusion with intact cells, or polycations, e.g., polybrene or polyomithine, may also be used. For various techniques for transforming mammalian cells, see Keown, et al., Methods in Enzymology 185: 527-37 (1990) and Mansour, et al., Nature 336(6197): 348-52 (1988).

Suitable host cells for cloning or expressing the nucleic acid (e.g., DNA) in the vectors herein include prokaryote, yeast, or higher eukaryote cells. Suitable prokaryotes include but are not limited to eubacteria, such as Gram-negative or Gram-positive organisms, for example, Enterobacteriacea such as E. coli. Various E. coli strains are publicly available, such as E. coli K12 strain MM294 (ATCC 3 1.446); E. coli X1 776 (ATCC 3 1.537); E. coli strain W3 110 (ATCC 27.325) and K5 772 (ATCC 53.635). Other suitable prokaryotic host cells include Enterobacteriaceae such as Escherichia, e.g. E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 4 1 P disclosed in DD266,7 10, published Apr. 12, 1989), Pseudomonas such as P. aeruginosa, and Streptomyces. These examples are illustrative rather than limiting. Strain W3110 is one particularly preferred host or parent host because it is a common host strain for recombinant DNA product fermentations. Preferably, the host cell secretes minimal amounts of proteolytic enzymes. For example, strain W3110 may be modified to effect a genetic mutation in a gene encoding proteins endogenous to the host, with examples of such hosts including E. coli W3110 strain 1A2, which has the complete genotype tonAD; E. coli W3110 strain 9E4, which has the complete genotype tonAD ptr3; E. coli W3110 strain 27C7 (ATCC 55,244), which has the complete genotype tonAD ptr3 phoADE15 D(argF-lac)169 ompTD degP41kan^{R′}; E. coli W3110 strain 37D6, which has the complete genotype tonAD ptr3 phoADE15 D(argF-lac)169 ompTD degP41kan^{R} rbs7D ilvG; E. coli W3110 strain 40B4, which is strain 37D6 with a non-kanamycin resistant degP deletion mutation; and an E. coli strain having mutant periplasmic protease as disclosed in U.S. Pat. No. 4,946,783 issued Aug. 7, 1990. Alternatively, in vivo methods of cloning, e.g., PCR or other nucleic acid polymerase reactions, are suitable.

In addition to prokaryotes, eukaryotic microbes such as filamentous fungi or yeast are suitable cloning or expression hosts for LP vectors. Saccharomyces cerevisiae is a commonly used lower eukaryotic host microorganism. Others include Schizosaccharomyces pombe [Beach and Nurse, Nature 290: 140-3 (1981); EP 139,383 published May 2, 1995]; Muyveromyces hosts [U.S. Pat. No. 4,943,529; Fleer, et al., Bio/Technology 9(10): 968-75 (1991)] such as, e.g., K. lactis (MW98-8C, CBS683, CBS4574) [de Louvencourt, et al., J. Bacteriol. 154(2): 737-42 (1983)1; K. fiagilis (ATCC 12,424), K. bulgaricus (ATCC 16,045), K. wickeramii (ATCC 24,178), K. waltii (ATCC 56,500), K. drosophilarum (ATCC 36,906) [Van den Berg, et al., Bio/Technology 8(2): 135-9 (1990)]; K. thermotolerans, and K. marxianus; yarrowia (EP 402,226); Pichia pastoris (EP 183,070) [Sreekrishna, et al., J. Basic Microbiol. 28(4): 265-78 (1988)]; Candida; Trichoderma reesia (EP 244,234); Neurospora crassa [Case, et al., Proc. Natl. Acad. Sci. USA 76(10): 5259-63 (1979)]; Schwanniomyces such as Schwanniomyces occidentulis (EP 394,538 published Oct. 31, 1990); and filamentous fungi such as, e.g., Neurospora, Penicillium, Tolypocladium (WO 91/00357 published Jan. 10, 1991), and Aspergillus hosts such as A. nidulans [Ballance, et al., Biochem. Biophys. Res. Comm. 112(1): 284-9 (1983)]; Tilburn, et al., Gene 26(2-3): 205-21 (1983); Yelton, et al., Proc. Natl. Acad. Sci. USA 81(5): 1470-4 (1984)] and A. niger [Kelly and Hynes, EMBO J. 4(2): 475-9 (1985)]. Methylotropic yeasts are selected from the genera consisting of Hansenula, Candida, Kloeckera, Pichia, Saccharomyces, Torulopsis, and Rhodotoruia. A list of specific species that are exemplary of this class of yeast may be found in C. Antony, The Biochemistry of Methylotrophs 269 (1982).

Suitable host cells for the expression of glycosylated LP polypeptide are derived from multicellular organisms. Examples of invertebrate cells include insect cells such as Drosophila S2 and Spodoptera Sp, Spodoptera high5 as well as plant cells. Examples of useful mammalian host cell lines include Chinese hamster ovary (CHO) and COS cells. More specific examples include monkey kidney CV1 line transformed by SV40 (COS-7, ATCC CRL 1651); human embryonic kidney line [293 or 293 cells subcloned for growth in suspension culture, Graham, et al., J. Gen Virol., 36(1): 59-74 (1977)]; Chinese hamster ovary cells/-DHFR [CHO, Urlaub and Chasin, Proc. Natl. Acad. Sci. USA, 77(7): 4216-20 (1980)]; mouse sertoli cells [TM4, Mather, Biol. Reprod. 23(1):243-52 (1980)]; human lung cells (W138. ATCC CCL 75); human liver cells (Hep G2, HB 8065); and mouse mammary tumor (MMT 060562, ATCC CCL51). The selection of the appropriate host cell is deemed to be within the skill in the art.

LP polypeptide may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector, or it may be a part of the LP226, LP233, or LP236-encoding DNA that is inserted into the vector. The signal sequence may be a prokaryotic signal sequence selected, for example, from the group of the alkaline phosphatase, penicillinase, ipp, or heat-stable enterotoxin II leaders. For yeast secretion the signal sequence may be, e.g., the yeast invertase leader, alpha factor leader (including Saccharomyces and Kluyveromyces cc-factor leaders, the latter described in U.S. Pat. No. 5,010,182), or acid phosphatase leader, the C. albicans glucoamylase leader (EP 362,179), or the signal described in WO 90/13646. In mammalian cell expression, mammalian signal sequences may be used to direct secretion of the protein, such as signal sequences from secreted polypeptides of the same or related species as well as viral secretory leaders.

Both expression and cloning vectors contain a nucleic acid sequence that enables the vector to replicate in one or more selected host cells. Such sequences are well known for a variety of bacteria, yeast, and viruses. The origin of replication from the plasmid pBR322 is suitable for most Gram-negative bacteria, the 2 u plasmid origin is suitable for yeast, and various viral origins (SV40, polyoma, adenovirus, VSV or BPV) are useful for cloning vectors in mammalian cells.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

An example of suitable selectable markers for mammalian cells are those that enable the identification of cells competent to take up the LP polypeptide-encoding nucleic acid, such as DHFR or thymidine kinase. An appropriate host cell when wild-type DHFR is employed is the CHO cell line deficient in DHFR activity, prepared and propagated as described Urlaub and Chasin, Proc. Natl. Acad. Sci. USA, 77(7): 4216-20 (1980). A suitable selection gene for use in yeast is the trpl gene present in the yeast plasmid YRp7 [Stinchcomb, et al., Nature 282(5734): 39-43 (1979); Kingsman, et al., Gene 7(2): 141-52 (1979); Tschumper, et al., Gene 10(2): 157-66 (1980)]. The trpl gene provides a selection marker for a mutant strain of yeast lacking the ability to grow in tryptophan, for example, ATCC No. 44076 or PEPC1 [Jones, Genetics 85: 23-33 (1977)].

Expression and cloning vectors usually contain a promoter operably linked to the LP226, LP233, or LP236-encoding nucleic acid sequence to direct mRNA synthesis. Promoters recognized by a variety of potential host cells are well known. Promoters suitable for use with prokaryotic hosts include the P-lactamase and lactose promoter systems [Chang, et al., Nature 275(5681): 617-24 (1978); Goeddel, et al., Nature 281(5732): 544-8 (1979)], alkaline phosphatase, a tryptophan (up) promoter system [Goeddel, Nucleic Acids Res. 8(18): 4057-74 (1980); EP 36,776 published Sep. 30, 1981], and hybrid promoters such as the tat promoter [deBoer, et al., Proc. Natl. Acad. Sci. USA 80(1): 21-5 (1983)]. Promoters for use in bacterial systems also will contain a Shine-Dalgarno (S.D.) sequence operably linked to the DNA encoding LP polypeptide.

Examples of suitable promoting sequences for use with yeast hosts include the promoters for 3-phosphoglycerate kinase [Hitzeman, et al., J. Biol. Chem. 255(24): 12073-80 (1980)] or other glycolytic enzymes [Hess, et al., J. Adv. Enzyme Reg. 7: 149 (1968); Holland, Biochemistry 17(23): 4900-7 (1978)], such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657. LP transcription from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

Transcription of a DNA encoding LP polypeptide by higher eukaryotes may be increased by inserting an enhancer sequence into the vector. Enhancers are cis-acting elements of DNA, usually about from 10 to 300 bp, that act on a promoter to increase its transcription. Many enhancer sequences are now known from mammalian genes (globin, elastase, albumin, alpha-ketoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. The enhancer may be spliced into the vector at a position 5′ or 3′ to the LP226, LP233, or LP236 coding sequence but is preferably located at a site 5′ from the promoter.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and occasionally 3′ untranslated regions of eukaryotic or viral DNAs or cDNAs. These regions contain nucleotide segments transcribed as polyadenylated fragments in the untranslated portion of the mRNA encoding LP polypeptide.

Gene amplification and/or expression may be measured in a sample directly, for example, by conventional Southern blotting, Northern blotting to quantitate the transcription of mRNA [Thomas, Proc. Natl. Acad. Sci. USA 77(9): 5201-5 (1980)], dot blotting (DNA analysis), or in situ hybridization, using an appropriately labeled probe, based on the sequences provided herein. Alternatively, antibodies may be employed that can recognize specific duplexes, including DNA duplexes, RNA duplexes, and DNA-RNA hybrid duplexes or DNA-protein duplexes. The antibodies in turn may be labeled and the assay may be carried out where the duplex is bound to a surface, so that upon the formation of duplex on the surface, the presence of antibody bound to the duplex can be detected.

Gene expression, alternatively, may be measured by immunological methods, such as immunohistochemical staining of cells or tissue sections and assay of cell culture or body fluids, to quantitate directly the expression of gene product. Antibodies useful for immunohistochemical staining and/or assay of sample fluids may be either monoclonal or polyclonal and may be prepared in any mammal. Conveniently, the antibodies may be prepared against a native sequence provided herein or against exogenous sequence fused to LP226, LP233, or LP236-encoding DNA and encoding a specific antibody epitope.

Forms of LP polypeptide may be recovered from culture medium or from host cell lysates. If membrane-bound, it can be released from the membrane using a suitable detergent solution (e.g., Triton-X 100) or by enzymatic cleavage. Cells employed in expression of LP polypeptide can be disrupted by various physical or chemical means, such as freeze-thaw cycling, sonication, mechanical disruption, or cell lysing agents.

It may be desireable to purify LP polypeptide from recombinant cell proteins or polypeptides. The following procedures are exemplary of suitable purification procedures: by fractionation on an ion-exchange column; ethanol precipitation; reversed-phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of LP polypeptide. Various methods of protein purification may be employed and such methods are known in the art and described, for example, in Deutscher, Methods in Enzymology 182: 83-9 (1990) and Scopes, Protein Purification: Principles and Practice, Springer-Verlag, NY (1982). The purification step(s) selected will depend, for example, on the nature of the production process used and the particular LP polypeptide produced.

Nucleotide sequences (or their complement) encoding LP polypeptides have various applications in the art of molecular biology, including uses as hybridization probes, in chromosome and gene mapping and in the generation of antisense RNA and DNA. LP polypeptide-encoding nucleic acid will also be useful for the preparation of LP polypeptide by the recombinant techniques described herein.

The full-length LP polypeptide-encoding nucleotide sequence (SEQ ID NO:1, 3, 5, respectively), or portions thereof, may be used as hybridization probes for a cDNA library to isolate the full-length LP226, LP233, or LP236 gene or genomic sequences including promoters, enhancer elements and introns of native sequence LP226, LP233, or LP236-encoding DNA or to isolate still other genes (for instance, those encoding naturally-occurring variants of LP polypeptide, or the same from other species) which have a desired sequence identity to the LP nucleotide sequence disclosed in SEQ ID NO:1, 3, 5, respectively. Hybridization techniques are well known in the art, some of which are described in further detail in the Examples below.

Other useful fragments of the LP nucleic acids include antisense or sense oligonucleotides comprising a single-stranded nucleic acid sequence (either RNA or DNA) capable of binding to target LP mRNA (sense) of LP DNA (antisense) sequences. Antisense or sense oligonucleotides, according to the present invention, comprise a fragment of the coding region of LP DNA. Such a fragment generally comprises at least about 14 nucleotides, preferably from about 14 to 30 nucleotides. The ability to derive an antisense or a sense oligonucleotide, based upon a cDNA sequence encoding a given protein is described in, for example, Stein and Cohen, Cancer Res 48(10): 2659-68 (1988) and van der Krol, et al., Bio/Techniques 6(10): 958-76 (1988).

Binding of antisense or sense oligonucleotides to target nucleic acid sequences results in the formation of duplexes that block transcription or translation of the target sequence by one of several means, including enhanced degradation of the duplexes, premature termination of transcription or translation, or by other means. The antisense oligonucleotides thus may be used to block expression of LP polypeptide. Antisense or sense oligonucleotides further comprise oligonucleotides having modified sugar-phosphodiester backbones (or other sugar linkages, such as those described in WO 91/06629) and wherein such sugar linkages are resistant to endogenous nucleases. Such oligonucleotides with resistant sugar linkages are stable in vivo (i.e., capable of resisting enzymatic degradation) but retain sequence specificity to be able to bind to target nucleotide sequences.

Other examples of sense or antisense oligonucleotides include those oligonucleotides which are covalently linked to organic moieties, such as those described in WO 90/10448, and other moieties that increase affinity of the oligonucleotide for a target nucleic acid sequence, such poly-L-lysine. Further still, intercalating agents, such as ellipticine, and alkylating agents or metal complexes may be attached to sense or antisense oligonucleotides to modify binding specificities of the antisense or sense oligonucleotide for the target nucleotide sequence.

Antisense or sense oligonucleotides may be introduced into a cell containing the target nucleic acid sequence by any gene transfer method, including, for example, calcium phosphate mediated DNA transfection, electroporation, or by using gene transfer vectors such as Epstein-Barr virus. In a preferred procedure, an antisense or sense oligonucleotide is inserted into a suitable retroviral vector. A cell containing the target nucleic acid sequence is contacted with the recombinant retroviral vector, either in vivo or ex vivo. Suitable retroviral vectors include, but are not limited to, those derived from the murine retrovirus M-MSV, N2 (a retrovirus derived from M-MuLV), or the double copy vectors designated CDTSA, CTSB and DCTSC (see WO 90/13641).

Alternatively, a sense or an antisense oligonucleotide may be introduced into a cell containing the target nucleic acid sequence by formation of an oligonucleotide-lipid complex, as described in WO 90/10448. The sense or antisense oligonucleotide-lipid complex is preferably dissociated within the cell by an endogenous lipase.

When the coding sequences for LP polypeptide encode a protein which binds to another protein, the LP polypeptide can be used in assays to identify the other proteins or molecules involved in the binding interaction. By such methods, inhibitors of the receptor/ligand binding interaction can be identified. Proteins involved in such binding interactions can also be used to screen for peptide or small molecule inhibitors or agonists of the binding interaction. Also, the receptor LP polypeptide can be used to isolate correlative ligand(s). Screening assays can be designed to find lead compounds that mimic the biological activity of a native LP polypeptide or a receptor for LP polypeptide. Such screening assays will include assays amenable to high-throughput screening of chemical libraries, making them particularly suitable for identifying small molecule drug candidates. Small molecules contemplated include synthetic organic or inorganic compounds. The assays can be performed in a variety of formats, including protein-protein binding assays, biochemical screening assays, immunoassays and cell based assays, which are well characterized in the art.

Nucleic acids which encode LP polypeptides or any of its modified forms can also be used to generate either transgenic animals or “knock out” animals which, in turn, are useful in the development and screening of therapeutically useful reagents. Methods for generating transgenic animals, particularly animals such as mice or rats, have become conventional in the art and are described, for example, in U.S. Pat. Nos. 4,736,866 and 4,870,009. Typically, particular cells would be targeted for an LP226, LP233, or LP236 transgene incorporation with tissue-specific enhancers. Transgenic animals that include a copy of a transgene introduced into the germ line of the animal at an embryonic stage can be used to examine the effect of increased expression of DNA encoding LP polypeptide. Such animals can be used as tester animals for reagents thought to confer protection from, for example, pathological conditions associated with its overexpression. In accordance with this facet of the invention, an animal is treated with the reagent and a reduced incidence of the pathological condition, compared to untreated animals bearing the transgene, would indicate a potential therapeutic intervention for the pathological condition.

Alternatively, non-human homologs of LP226, LP233, or LP236 can be used to construct a “knock out” animal which has a defective or altered gene encoding LP polypeptide as a result of homologous recombination between the endogenous gene encoding LP polypeptide and altered genomic DNA introduced into an embryonic cell of the animal. For example, cDNA encoding LP polypeptide can be used to clone genomic DNA encoding LP polypeptide in accordance with established techniques. A portion of the genomic DNA encoding LP226, LP233, or LP236 can be deleted or replaced with another gene, such as a gene encoding a selectable marker which can be used to monitor integration. Typically, several kilobases of unaltered flanking DNA (both at the 5′ and 3′ ends) are included in the vector [see, e.g., Thomas and Capecchi, Cell 51(3): 503-12 (1987) for a description of homologous recombination vectors]. The vector is introduced into an embryonic stem cell line (e.g., by electroporation), and cells in which the introduced DNA has homologously recombined with the endogenous DNA are selected [see, e.g., Li, et al., Cell 69(6): 915-26 (1992)]. The selected cells are then injected into a blastocyst of an animal (e.g., a mouse or rat) to form aggregation chimeras [see, e.g., Bradley, Teratocarcinomas and Embryonic Stem Cells: A Practical Approach, E. J. Robertson, ed. (IRL, Oxford, 1987), pp. 113-152]. A chimeric embryo can then be implanted into a suitable pseudopregnant female foster animal and the embryo brought to term to create a “knock out” animal. Progeny harboring the homologously recombined DNA in their germ cells can be identified by standard techniques and used to breed animals in which all cells of the animal contain the homologously recombined DNA. Knockout animals can be characterized, for instance, for their ability to defend against certain pathological conditions and for their development of pathological conditions due to absence of LP226, LP233, or LP236.

LP polypeptide transgenic non-human mammals are useful as animal models in both basic research and drug development endeavors. Transgenic animals carrying at least one LP polypeptide or nucleic acid can be used to test compounds or other treatment modalities which may prevent, suppress, or cure a pathology or disease associated with at least one of the above mentioned activities. Such transgenic animals can also serve as a model for the testing of diagnostic methods for those same diseases. Furthermore, tissues derived from LP polypeptide transgenic non-human mammals are useful as a source of cells for cell culture in efforts to develop in vitro bioassays to identify compounds that modulate their activity or dependent signaling. Accordingly, another aspect of the present invention contemplates a method of identifying compounds efficacious in the treatment of at least one previously described disease or pathology associated with an LP polypeptide activity. A non-limiting example of such a method comprises:

a) generating an LP polypeptide transgenic non-human animal which is, as compared to a wild-type animal, pathologically distinct in some detectable or measurable manner from wild-type version of said non-human mammal;

b) exposing said transgenic animal to a compound, and;

c) determining the progression of the pathology in the treated transgenic animal, wherein an arrest, delay, or reversal in disease progression in transgenic animal treated with said compound as compared to the progression of the pathology in an untreated control animals is indicative that the compound is useful for the treatment of said pathology.

Another embodiment of the present invention provides a method of identifying compounds capable of inhibiting LP polypeptide activity in vivo and/or in vitro wherein said method comprises:

a) administering an experimental compound to an LP polypeptide transgenic non-human animal, or tissues derived therefrom, exhibiting one or more physiological or pathological conditions attributable to the overexpression of an LP polypeptide transgene; and

b) observing or assaying said animal and/or animal tissues to detect changes in said physiological or pathological condition or conditions.

Another embodiment of the invention provides a method for identifying compounds capable of overcoming deficiencies in LP226, LP233, or LP236 activity in vivo or in vitro wherein said method comprises:

a) administering an experimental compound to an LP226, LP233, or LP236 transgenic non-human animal, or tissues derived therefrom, exhibiting one or more physiological or pathological conditions attributable to the disruption of the endogenous LP226, LP233, or LP236 gene; and

b) observing or assaying said animal and/or animal tissues to detect changes in said physiological or pathological condition or conditions.

Various means for determining a compound's ability to modulate LP polypeptide in the body of the transgenic animal are consistent with the invention. Observing the reversal of a pathological condition in the transgenic animal after administering a compound is one such means. Another more preferred means is to assay for markers of LP polypeptide activity in the blood of a transgenic animal before and after administering an experimental compound to the animal. The level of skill of an artisan in the relevant arts readily provides the practitioner with numerous methods for assaying physiological changes related to therapeutic modulation of LP polypeptide activity.

“Gene therapy” includes both conventional gene therapy, where a lasting effect is achieved by a single treatment, and the administration of gene therapeutic agents, which involves the one time or repeated administration of a therapeutically effective DNA or mRNA. Antisense RNAs and DNAs can be used as therapeutic agents for blocking the expression of certain genes in vivo. It has already been shown that short antisense oligonucleotides can be imported into cells where they act as inhibitors, despite their low intracellular concentrations caused by their restricted uptake by the cell membrane [Zamecnik, et al., Proc. Natl. Acad. Sci. USA 83(12): 4143-6 (1986)1. The oligonucleotides can be modified to enhance their uptake, e.g., by substituting their negatively charged phosphodiester groups with uncharged groups.

There are a variety of techniques available for introducing nucleic acids into viable cells. The techniques vary depending upon whether the nucleic acid is transferred into cultured cell in vitro or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically, retroviral) vectors and viral coat protein-liposome mediated transfection [Dzau, et al., Trends in Biotechnology 11(5): 205-10 (1993)]. In some situations it is desirable to provide the nucleic acid source with an agent that targets the target cells, such as an antibody specific for a cell surface membrane protein or the target cell, a ligand for a receptor on the target cells, etc. Where liposomes are employed, proteins which bind to a cell surface membrane protein associated with endocytosis may by used for targeting and/or to facilitate uptake, e.g., capsid proteins or fragments thereof trophic for a particular cell type, antibodies for proteins which undergo internalization in cycling, proteins that target intracellular localization and enhance intracellular half-life. The technique of receptor-mediated endocytosis is described, for example by Wu, et al., J. Biol. Chem. 262(10): 4429-32 (1987); and Wagner, et al., Proc. Natl. Acad. Sci. USA 87(9): 3410-4 (1990). For a review of gene marking and gene therapy protocols, see Anderson, Science 256(5058): 808-13 (1992).

The nucleic acid molecules encoding LP polypeptide or fragments thereof described herein are useful for chromosome identification. In this regard, there exists an ongoing need to identity new chromosome markers, since relatively few chromosome marking reagents, based upon actual sequence data, are presently available. Each LP nucleic acid molecule of the present invention can be used as a chromosome marker. The LP polypeptide encoding nucleic acid or fragments thereof can also be used for localization in the 11q14 chromosomal region.

The present invention further provides anti-LP antibodies. Exemplary antibodies include polyclonal, monoclonal, humanized, bispecific, and heteroconjugate antibodies.

The anti-LP antibodies of the present invention may comprise polyclonal antibodies. Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include the LP polypeptide or a fusion protein thereof. It may be useful to conjugate the immunizing agent to a protein known to be immunogenic in the mammal being immunized. Examples of such immunogenic proteins include, but are not limited to, keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

The anti-LP antibodies may, alternatively, be monoclonal antibodies. Monoclonal antibodies may be prepared using hybridoma methods, such as those described by Kohler and Mistein, Nature 256(5517): 495-7 (1975). In a hybridoma method, a mouse, hamster, or other appropriate host animal is typically immunized with an immunizing agent to elicit lymphocytes that produce or are capable of producing antibodies that will specifically bind to the immunizing agent. Alternatively, the lymphocytes may be immunized in vitro.

The immunizing agent will typically include LP polypeptide or a fusion protein thereof. Generally, either peripheral blood lymphocytes (“PBLs”) are used, if cells of human origin are desired, or spleen cells or lymph node cells are used, if non-human mammalian sources are desired. The lymphocytes are then fused with an immortalized cell line using a suitable fusing agent, such as polyethylene glycol, to form a hybridoma cell [Goding, Monoclonal Antibodies: Principles and Practice, Academic Press, (1986) pp. 59-103]. Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which prevents the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif., and the American Type Culture Collection, Rockville, Md. Human myeloma and mouse-human heteromyeloma cell lines also have been described for the production of human monoclonal antibodies [Kozbor, J. Immunol. 133(6): 3001-5 (1984); Brodeur, et al., Monoclonal Antibody Production Techniques and Applications, Marcel Dekker, Inc., NY, (1987) pp. 51-63].

The culture medium in which the hybridoma cells are cultured can then be assayed for the presence of monoclonal antibodies directed against LP polypeptide. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Rodbard, Anal. Biochem. 107(1): 220-39 (1980).

After the desired hybridoma cells are identified, the clones may be subcloned by limiting dilution procedures and grown by standard methods [Goding, Monoclonal Antibodies: Principles and Practice, Academic Press, (1986) pp. 59-103]. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies may also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences [U.S. Pat. No. 4,816,567; Morrison, et al., Proc. Natl. Acad. Sci. USA 81(21): 6851-5 (1984)] or by covalently joining to the immunoglobulin coding sequence all or part of the coding sequence for a non-immunoglobulin polypeptide. Such a non-immunoglobulin polypeptide can be substituted for the constant domains of an antibody of the invention or can be substituted for the variable domains of one antigen-combining site of an antibody of the invention to create a chimeric bivalent antibody.

The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly Fab fragments, can be accomplished using routine techniques known in the art.

The anti-LP antibodies of the invention may further comprise humanized antibodies or human antibodies. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementary-determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin, and all or substantially all of the FR regions are those of a human immunoglobulin consensus sequence. The humanized antibody optimally also will comprise at least a portion of an immunoglobulin constant region (Fc), typically that of a human immunoglobulin [Jones, et al., Nature 321(6069): 522-5 (1986); Riechmann, et al., Nature 332(6162): 323-7 (1988); and Presta, Curr. Op. Struct. Biol. 2: 593-6 (1992)].

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers [Jones, et al., Nature 321(6069): 522-5 (1986); Riechmann, et al., Nature 332(6162): 323-7 (1988); Verhoeyen, et al., Science 239(4847): 1534-6 (1988)], by substituting rodent CDRs or CDR sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol. 227(2): 381-8 (1992); Marks, et al., J. Mol. Biol. 222(3): 581-97 (1991)]. The techniques of Cole, et al. and Boerner, et al. are also available for the preparation of human monoclonal antibodies (Cole, et al., Monoclonal Antibodies and Cancer Therapy, Alan R. Liss, p. 77 (1985) and Boerner, et al., J. Immunol. 147(1): 86-95 (1991)]. Similarly, human antibodies can be made by introducing human immunoglobulin loci into transgenic animals, e.g., mice in which the endogenous immunoglobulin genes have been partially or complete inactivated. Upon challenge, human antibody production is observed, which closely resembles that seen in humans in all respects, including gene rearrangement, assembly and antibody repertoire. This approach is described, for example, in U.S. Pat. Nos. 5,545,807; 5,545,806; 5,569,825; 5,625,126; 5,633,425; 5,661,016, and in the following scientific publications: Marks, et al., Biotechnology 10(7): 779-83 (1992); Lonberg, et al., Nature 368(6474): 856-9 (1994); Morrison, Nature 368(6474): 812-3 (1994); Fishwild, et al., Nature Biotechnology 14(7): 845-51 (1996); Neuberger, Nature Biotechnology 14(7): 826 (1996); Lonberg and Huszar, Int. Rev. Immunol. 13(1): 65-93 (1995).

Bispecific antibodies are monoclonal, preferably human or humanized, antibodies that have binding specificities for at least two different antigens. In the present case, one of the binding specificities is for an LP226, LP233, or LP236 polypeptide, the other one is for any other antigen, and preferably for a cell-surface protein or receptor or receptor subunit. Methods for making bispecific antibodies are known in the art. Antibodies with more than two valencies are contemplated. For example, trispecific antibodies can be prepared [Tutt, et al., J. Immunol. 147(1): 60-9 (1991)].

Heteroconjugate antibodies are also within the scope of the present invention. Heteroconjugate antibodies are composed of two covalently joined antibodies. Such antibodies have, for example, been proposed to target immune system cells to unwanted cells [U.S. Pat. No. 4,676,980], and for treatment of HIV infection [WO 91/00360; WO 92/20373]. It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

The invention also pertains to immunoconjugates comprising an antibody conjugated to a cytotoxic agent such as a chemotherapeutic agent, toxin (e.g., an enzymatically active toxin of bacterial, fungal, plant or animal origin, or fragments thereof, or a small molecule toxin), or a radioactive isotope (i.e., a radioconjugate).

Conjugates of the antibody and cytotoxic agent are made using a variety of bifunctional protein coupling agents such as N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds, bis-diazonium derivatives (such as bis-2-diazoniumbenzoyl)-ethylenediamine)_{T} diisocyanates (such as tolylene 2,6-diisocyanate), and bioactive fluorine compounds. For example, a ricin immunotoxin can be prepared as described in Vitetta, et al., Science 238(4830): 1098-104 (1987). Carbon-14-labeled 1-isothiocyanatobenzyl-3-methyldiethylene triaminepentaacetic acid (MX-DTPA) is an exemplary chelating agent for conjugation of radionucleotide to the antibody.

In another embodiment, the antibody may be conjugated to a “receptor” (such as streptavidin) for utilization in tumor pretargeting wherein the antibody-receptor conjugate is administered to the patient, followed by removal of unbound conjugate from the circulation using a clearing agent, and then administration of a “ligand” (e.g., avidin) which is conjugated to a cytotoxic agent (e.g., a radionucleotide).

The antibodies disclosed herein may also be formulated as immunoliposomes. Liposomes containing the antibody are prepared by methods known in the art, such as described in Eppstein, et al., Proc. Natl. Acad. Sci. USA 82: 3688-92 (1985); Hwang, et al., Proc. Natl. Acad. Sci. USA 77(7): 4030-4 (1980); and U.S. Pat. Nos. 4,485,045 and 4,544,545. Liposomes with enhanced circulation time are disclosed in U.S. Pat. No. 5,013,556.

Particularly useful liposomes can be generated by the reversed phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin, et al., J. Biol. Chem. 257(1): 286-8 (1982) via a disulfide interchange reaction. A chemotherapeutic agent (such as Doxorubicin) is optionally contained within the liposome. See Gabizon, et al., J. National Cancer Inst. 81(19): 484-8 (1989).

Antibodies specifically binding LP226, LP233, or LP236 identified herein, as well as other molecules identified by the screening assays disclosed hereinbefore, can be administered for the treatment of various disorders in the form of pharmaceutical compositions.

If an LP226, LP233, or LP236 polypeptide is intracellular and whole antibodies are used as inhibitors, internalizing antibodies are preferred. However, lipofections or liposomes can also be used to deliver the antibody or an antibody fragment into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco, et al., Proc. Natl. Acad. Sci. USA 90(16): 7889-93 (1993).

The formulation herein may also contain more than one active compound as necessary for the particular indication being treated, preferably those with complementary activities that do not adversely affect each other. Alternatively, or in addition, the composition may comprise an agent that enhances its function, such as, for example, a cytotoxic agent, cytokines, chemotherapeutic agent, or growth-inhibitory agent. Such molecules are suitable present in combination in amounts that are effective for the purpose intended. The active ingredients may also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions. Such techniques are disclosed in Remington's Pharmaceutical Sciences, supra.

The formulations to be used for in vivo administration must be sterile. This is readily accomplished by filtration through sterile filtration membranes.

Sustained-release preparations may be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly-(2-hydroxyethylmethacrylate), or polyvinyl alcohol), polylactides (U.S. Pat. No. 3,773,919), copolymers of L-glutamic acid gamma-ethyl-L-glutamate, non-degradable ethylene-vinylacetate, degradable lactic acid-glycolic acid copolymers such as the LUPRON DEPOT™ (injectable microspheres composed of lactic acid-glycolic acid copolymer and leuprolide acetate), and poly-D-(−)3-hydroxylbutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37 degrees C., resulting in a loss of biological activity and possible changes in immunogenicity. Rational strategies can be devised for stabilization depending on the mechanisms involved. For example, if the aggregation mechanism is discovered to be intermolecular S—S bond formation through thiosulfide interchange, stabilization may be achieved by modifying sulfhydryl residues, lyophilizing from acidic solutions, controlling moisture content, using appropriate additives, and developing specific polymer matrix compositions.

The anti-LP antibodies of the present invention have various utilities. For example, such antibodies may be used in diagnostic assays for LP polypeptide, e.g., detecting expression in specific cells, tissues, or serum. Various diagnostic assay techniques known in the art may be used, such as competitive binding assays, direct or indirect sandwich assays and immunoprecipitation assays conducted in either heterogeneous or homogeneous phases [Zola, Monoclonal Antibodies: A Manual of Techniques, CRC Press, Inc. (1987) pp. 147-158]. The-antibodies used in the assays can be labeled with a detectable moiety. The detectable moiety should be capable of producing, either directly or indirectly, a detectable signal. For example, the detectable moiety may be a radioisotope, such as ³H, ¹⁴C, ³²P, ³⁵S, or ¹²⁵I, a fluorescent or chemiluminescent compound, such as fluorescein isothiocyanate, rhodamine, or luciferin, or an enzyme, such as alkaline phosphatase, beta-galactosidase or horseradish peroxidase. Any method known in the art for conjugating the antibody to the detectable moiety may be employed, including those methods described by Hunter, et al., Nature 144: 945 (1962); David, et al., Biochemistry 13(5): 1014-21 (1974); Pain, et al., J Immunol. Meth., 40(2): 219-30 (1981); and Nygren, J. Histochem. Cytochem. 30(5): 407-12 (1982).

Anti-LP antibodies also are useful for affinity purification from recombinant cell culture or natural sources. In this process, the antibodies are immobilized on a suitable support, such a Sephadex resin or filter paper, using methods well known in the art. The immobilized antibody is then contacted with a sample containing LP226, LP233, or LP236 to be purified, and thereafter the support is washed with a suitable solvent that will remove substantially all the material in the sample except the LP polypeptide, which is bound to the immobilized antibody. Finally, the support is washed with another suitable solvent that will release the desired polypeptide from the antibody.

This invention encompasses methods of screening compounds to identity those that mimic LP226, LP233, or LP236 (agonists) or prevent the effect of LP226, LP233, or LP236 (antagonists). Screening assays for antagonist drug candidates are designed to identity compounds that bind or complex with LP226, LP233, or LP236 encoded by the genes identified herein or otherwise interfere with the interaction of the encoded polypeptides with other cellular proteins. Such screening assays will include assays amenable to high-throughput screening of chemical libraries, making them particularly suitable for identifying small molecule drug candidates.

The assays can be performed in a variety of formats. In binding assays, the interaction is binding, and the complex formed can be isolated or detected in the reaction mixture. In a particular embodiment, LP226, LP233, or LP236 encoded by a gene identified herein or the drug candidate is immobilized on a solid phase, e.g., on a microtiter plate, by covalent or non-covalent attachments. Non-covalent attachment generally is accomplished by coating the solid surface with a solution of LP polypeptide and drying. Alternatively, an immobilized antibody, e.g., a monoclonal antibody, specific for the polypeptide to be immobilized can be used to anchor it to a solid surface. The assay is performed by adding the non-immobilized component, which may be labeled by a detectable label, to the immobilized component, e.g., the coated surface containing the anchored component. When the reaction is complete, the non-reacted components are removed, e.g., by washing, and complexes anchored on the solid surface are detected. When the originally non-immobilized component carries a detectable label, the detection of label immobilized on the surface indicates that complexing occurred. Where the originally non-immobilized component does not carry a label, complexing can be detected, for example, by using a labeled antibody specifically binding the immobilized complex.

If the candidate compound interacts with but does not bind to LP polypeptide, its interaction with that polypeptide can be assayed by methods well known for detecting protein-protein interactions. Such assays include traditional approaches, such as, e.g., crosslinking, co-immunoprecipitation, and co-purification through gradients or chromatographic columns. In addition, protein-protein interactions can be monitored through gradients or chromatographic columns. In addition, protein-protein interactions can be monitored by using a yeast-based genetic system described by Fields and co-workers [Fields and Song, Nature 340(6230): 245-6 (1989); Chien, et al., Proc. Natl. Acad. Sci. USA 88(21): 9578-82 (1991); Chevray and Nathans, Proc. Natl. Acad. Sci. USA 89(13): 5789-93 (1992)]. Many transcriptional activators, such as yeast GAL4, consist of two physically discrete modular domains, one acting as the DNA-binding domain, while the other functions as the transcription-activation domain. The yeast expression system described in the foregoing publications (generally referred to as the “two-hybrid system”) takes advantage of this property, and employs two hybrid proteins, one in which the target protein is fused to the DNA-binding domain of GAL4, and another in which candidate activating proteins are fused to the activation domain. The expression of GAL1-lacZ reporter gene under control of a GAL4-activated promoter depends on reconstitution of GAL4 activity via protein-protein interaction. Colonies containing interacting polypeptides are detected with chromogenic substrate for beta-galactosidase. A complete kit (MATCHMAKER™) for identifying protein-protein interactions between two specific proteins using the two-hybrid technique is commercially available from Clontech. This system can also be extended to map protein domains involved in specific protein interactions as well as to pinpoint amino acid residues that are crucial for these interactions.

Compounds that interfere with the interaction of a gene encoding LP polypeptide identified herein and other intra- or extracellular components can be tested as follows: usually a reaction mixture is prepared containing the product of the gene and the intra- or extracellular component under conditions and for a time allowing for the interaction and binding of the two products. To test the ability of a candidate compound to inhibit binding, the reaction is run in the absence and in the presence of the test compound. In addition, a placebo may be added to a third reaction mixture to serve as a positive control. The binding (complex formation) between the test compound and the intra- or extracellular component present in the mixture is monitored as described hereinabove. The formation of a complex in the control reaction(s) but not in the reaction mixture containing the test compound indicates that the test compound interferes with the interaction of the test compound and its reaction partner.

Antagonists may be detected by combining LP polypeptide and a potential antagonist with membrane-bound LP polypeptide receptors or recombinant receptors under appropriate conditions for a competitive inhibition assay. LP polypeptide can be labeled, such as by radioactivity, such that the number of LP polypeptide molecules bound to the receptor can be used to determine the effectiveness of the potential antagonist. The gene encoding the receptor can be identified by numerous methods known to those of skill in the art, for example, ligand panning and FACS sorting. See Coligan, et al., Current Protocols in Immunology 1(2): Chap. 5 (1991). Preferably, expression cloning is employed wherein polyadenylated RNA is prepared from a cell responsive to LP polypeptide, and a cDNA library created from this RNA is divided into pools and used to transfect COS cells or other cells that are not responsive to LP polypeptide. Transfected cells that are grown on glass slides are exposed to labeled LP polypeptide. The polypeptide can be labeled by a variety of means including iodination or inclusion of a recognition site for a site-specific protein kinase. Following fixation and incubation, the slides are subjected to autoradiographic analysis. Positive pools are identified and sub-pools are prepared and re-transfected using an interactive sub-pooling and re-screening process, eventually yielding a single clone that encodes the putative receptor.

As an alternative approach for receptor identification, labeled LP polypeptide can be photoaffinity-linked-with cell membrane or extract preparations that express the receptor molecule. Cross-linked material is resolved by PAGE and exposed to X-ray film. The labeled complex containing the receptor can be excised, resolved into peptide fragments, and subjected to protein micro-sequencing. The amino acid sequence obtained from micro-sequencing would be used to design a set of degenerate oligonucleotide probes to screen a cDNA library to identify the gene encoding the putative receptor.

In another assay for antagonists, mammalian cells or a membrane preparation expressing the receptor would be incubated with labeled LP polypeptide in the presence of the candidate compound. The ability of the compound to enhance or block this interaction could then be removed.

Alternatively, a potential antagonist may be a closely related protein, for example, a mutated form of LP polypeptide that recognizes the receptor but imparts no effect, thereby competitively inhibiting the action of the polypeptide.

Another potential LP antagonist is an antisense RNA or DNA construct prepared using antisense technology, where, e.g., an antisense RNA or DNA molecule acts to block directly the translation of mRNA by hybridizing to targeted mRNA and prevent its translation into protein. Antisense technology can be used to control gene expression through triple-helix formation or antisense DNA or RNA, both of which methods are based on binding of a polynucleotide to DNA or RNA. For example, the 5′ coding portion of the polynucleotide sequence, which encodes the mature LP polypeptide, is used to design an antisense RNA oligonucleotide sequence of about 10 to 40 base pairs in length. A DNA oligonucleotide is designed to be complementary to a region of the gene involved in transcription [triple helix; see Lee, et al., Nucl. Acids Res 6(9): 3073-91 (1979); Cooney, et al., Science 241(4864): 456-9 (1988); Beal and Dervan, Science 251(4999): 1360-3 (1991)], thereby preventing transcription and production of LP polypeptide. The antisense RNA oligonucleotide hybridizes to the mRNA in vivo and blocks translation of the mRNA molecules [antisense; see Okano, J. Neurochem. 56(2): 560-7 (1991); Oligodeoxynucleotides as Antisense Inhibitors of Gene Expression (CRC Press: Boca Raton, Fla. 1988)]. The oligonucleotides described above can also be delivered to cells such that the antisense RNA or DNA may be expressed in vivo to inhibit production of LP polypeptide. When antisense DNA is used, oligodeoxyribonucleotides derived from the translation-initiation site, e.g., between about −10 and +10 positions of the target gene nucleotide sequence, are preferred.

Potential antagonists include small molecules that bind to the active site, the receptor binding site, or growth factor or other relevant binding site of LP polypeptide, thereby blocking the normal biological activity of LP polypeptide. Examples of small molecules include, but are not limited to, small peptides or peptide-like molecules, preferably soluble peptides, and synthetic non-peptidyl organic or inorganic compounds.

Ribozymes are enzymatic RNA molecules capable of catalyzing the specific cleavage of RNA. Ribozymes act by sequence-specific hybridization to the complementary target RNA, followed by endonucleolytic cleavage. Specific ribozyme cleavage sites within a potential RNA target can be identified by known techniques. For further details, see, e.g., Rossi, Current Biology 4(5): 469-71 (1994) and PCT publication No. WO 97/33551 (published Sep. 18, 1997).

Nucleic acid molecules in triple-helix formation used to inhibit transcription should be single-stranded and composed of deoxynucleotides. The base composition of these oligonucleotides is designed such that it promotes triple-helix formation via Hoogsteen base-pairing rules, which generally require sizeable stretches of purines or pyrimidines on one strand of a duplex. For further details see, e.g., PCT publication No. WO 97/33551, supra.

Another use of the compounds of the invention (e.g., LP polypeptide variants and anti-LP antibodies) described herein is to help diagnose whether a disorder is driven to some extent by LP polypeptide modulated signaling.

A diagnostic assay to determine whether a particular disorder is driven by LP polypeptide signaling can be carried out using the following steps:

a) culturing test cells or tissues expressing LP polypeptide;

b) administering a compound which can inhibit LP polypeptide modulated signaling; and

c) measuring the LP polypeptide mediated phenotypic effects in the test cells.

The steps can be carried out using standard techniques in light of the present disclosure. Appropriate controls take into account the possible cytotoxic effect of a compound, such as treating cells not associated with a cell proliferative disorder (e.g., control cells) with a test compound and can also be used as part of the diagnostic assay. The diagnostic methods of the invention involve the screening for agents that modulate the effects of LP polypeptide-associated disorders.

The LP polypeptide antagonists or agonists can be employed as therapeutic agents. Such therapeutic agents are formulated according to known methods to prepare pharmaceutically useful compositions, whereby the LP polypeptide antagonist or agonist thereof is combined in a mixture with a pharmaceutically acceptable carrier.

In the case of LP polypeptide antagonist or agonist antibodies, if the protein encoded by the amplified gene is intracellular and whole antibodies are used as inhibitors, internalizing antibodies are preferred. However, lipofections or liposomes can also be used to deliver the antibody, or an antibody fragment, into cells. Where antibody fragments are used, the smallest inhibitory fragment which specifically binds to the binding domain of the target protein is preferred. For example, based upon the variable region sequences of an antibody, peptide molecules can be designed which retain the ability to bind the target protein sequence. Such peptides can be synthesized chemically and/or produced by recombinant DNA technology [see, e.g., Marasco, et al., Proc. Natl. Acad. Sci. USA 90(16): 7889-93 (1993)].

Therapeutic formulations are prepared for storage by mixing the active ingredient having the desired degree of purity with optional pharmaceutically acceptable carriers, excipients or stabilizers [Remington's Pharmaceutical Sciences 16th edition (1980)], in the form of lyophilized formulations or aqueous solutions.

The formulation herein may also contain more than one active compound as necessary for the particular indication being treated, preferably those with complementary activities that do not adversely affect each other. Such molecules are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition (1980).

The formulations to be used for in vivo administration must be sterile. This is readily accomplished by filtration through sterile filtration membranes.

Therapeutic compositions herein generally are placed into a container having a sterile access port, for example, and intravenous solution bag or vial having a stopper pierceable by a hypodermic injection needle.

Sustained-release preparations may be prepared. Suitable examples of sustained-release preparations include semipermeable matrices of solid hydrophobic polymers containing the therapeutic agent(s), which matrices are in the form of shaped articles, e.g., films, or microcapsules. Examples of sustained-release matrices include polyesters, hydrogels [for example, poly-(2-hydroxyethylmethacrylate), or polyvinyl alcohol], polylactides, copolymers of L-glutamic acid and gamma-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. Microencapsulation of recombinant proteins for sustained release has been successfully performed with human growth hormone (rhGH), interferon, and interleukin-2. Johnson, et al., Nat. Med. 2(7): 795-9 (1996); Yasuda, et al., Biomed. Ther. 27: 1221-3 (1993); Hora, et al., Bio/Technology 8(8): 755-8 (1990); Cleland, “Design and Production of Single Immunization Vaccines Using Polylactide Polyglycolide Microsphere Systems” in Vaccine Design: The Subunit and Adjuvant Approach, Powell and Newman, Eds., Plenum Press, NY, 1995, pp. 439-462 WO 97/03692; WO 96/40072; WO 96/07399; and U.S. Pat. No. 5,654,010.

The sustained-release formulations of these proteins may be developed using polylactic-coglycolic acid (PLGA) polymer due to its biocompatibility and wide range of biodegradable properties. The degradation products of PLGA, lactic and glycolic acids, can be cleared quickly within the human body. Moreover, the degradability of this polymer can be adjusted from months to years depending on its molecular weight and composition. See Lewis, “Controlled release of bioactive agents from lactide/glycolide polymer” in Biodegradable Polymers as Drug Delivery Systems (Marcel Dekker; New York, 1990), M. Chasin and R. Langer (Eds.) pp. 1-41.

While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37 degrees C., resulting in a loss of biological activity and possible changes in immunogenicity.

It is contemplated that the compounds, including, but not limited to, antibodies, small organic and inorganic molecules, peptides, antisense molecules, ribozymes, etc., of the present invention may be used to treat various conditions including those characterized by overexpression and/or activation of the disease-associated genes identified herein. Exemplary conditions or disorders to be treated with such compounds include osteosarcoma, prostate cancer, breast cancer, renal carcinoma, thyroid carcinoma, multiple myeloma, lung cancer, salivary gland mucoepidermoid carcinoma, chronic lymphocytic leukemia, T cell lymphoblastic leukemia, chronic ulcers, burns, bone fractures, nerve tissue damage, spinal cord injuries, schizophrenia, tuberous sclerosis, prepubertal periodontitis, Papillon-Lefevre syndrome, achondroplasia, cleidocranial dysostosis, enchondromatosis, fibrous dysplasia, Gaucher's disease, hypophosphatemic rickets, Marfan's disease, multiple hereditary exotoses, neurofibromatosis, osteogenesis imperfecta, osteopetrosis, osteopoikilosis, sclerotic lesions, fractures, pseudoartritis, pyogenic osteomyelitis, anemia, steroid usage, conditions caused by heparin, bone marrow disorders, scurvy, malnutrition, calcium deficiency, idiopathic osteoporosis, congenital osteopenia or osteoporosis, chronic liver disease, oligomenorrhea, amenorrhea, diabetes mellitus, hyperthyroidism, Cushing's disease, acromegaly, hypogonadism, reflex sympathetic dystrophy syndrome, transient regional osteoporosis, osteomalacia, cardiovascular disease, kidney fibrosis, cirrhosis, idiopathic lung fibrosis, chronic asbestosis, pneumonitis, acute respiratory distress syndrome (ARDS), cardiac fibrosis, atherosclerosis, restenosis, macular degeneration, retinal and vitreal retinopathy, keloids, Crohn's disease, valvular ossification, Alzheimer's disease, Parkinson's disease, Huntington's disease, and multiple sclerosis.

The active agents of the present invention, e.g., antibodies, are administered to a mammal, preferably a human, in accord with known methods, such as intravenous administration as a bolus or by continuous infusion over a period of time, by intramuscular, intraperitoneal, intracerebral, intracerebrospinal, subcutaneous, intraarticular, intrasynovial, intrathecal, intraoccular, intralesional, oral, topical, inhalation, pulmonary, and/or through sustained release.

Other therapeutic regimens may be combined with the administration of LP polypeptide antagonists or antagonists, anti-cancer agents, e.g., antibodies of the instant invention.

For the prevention or treatment of disease, the appropriate dosage of an active agent, (e.g., an antibody) will depend on the type of disease to be treated, as defined above, the severity and course of the disease, whether the agent is administered for preventive or therapeutic purposes, previous therapy, the patient's clinical history and response to the agent, and the discretion of the attending physician. The agent is suitably administered to the patient at one time or over a series of treatments.

Dosages and desired drug concentration of pharmaceutical compositions of the present invention may vary depending on the particular use envisioned. The determination of the appropriate dosage or route of administration is well within the skill of an ordinary artisan. Animal experiments provide reliable guidance for the determination of effective does for human therapy. Interspecies scaling of effective doses can be performed following the principles laid down by Mordenti and Chappell, “The Use of Interspecies Scaling in Toxicokinetics,” in Toxicokinetics and New Drug Development, Yacobi, et al., Eds., Pergamon Press, NY 1989, pp.4246.

When in vivo administration of LP polypeptide or agonist or antagonist thereof is employed, normal dosage amounts may vary from about 1 ng/kg up to 100 mg/kg of mammal body weight or more per day, preferably about 1 pg/kg/day up to 100 mg/kg of mammal body weight or more per day, depending upon the route of administration. Guidance as to particular dosages and methods of delivery is provided in the literature; see, for example, U.S. Pat. No. 4,657,760, 5,206,344 or 5,225,212. It is within the scope of the invention that different formulations will be effective for different treatment compounds and different disorders, that administration targeting one organ or tissue, for example, may necessitate delivery in a manner different from that to another organ or tissue. Moreover, dosages may be administered by one or more separate administrations or by continuous infusion. For repeated administrations over several days or longer, depending on the condition, the treatment is sustained until a desired suppression of disease symptoms occurs. However, other dosage regimens may be useful. The progress of this therapy is easily monitored by conventional techniques and assays.

In another embodiment of the invention, an article of manufacture containing materials useful for the diagnosis or treatment of the disorders described above is provided. The article of manufacture comprises a container and a label. Suitable containers include, for example, bottles, vials, syringes, and test tubes. The containers may be formed from a variety of materials such as glass or plastic. The container holds a composition which is effective for diagnosing or treating the condition and may have a sterile access port (for example, the container may be an intravenous solution bag or a vial having a stopper pierceable by a hypodermic injection needle). The active agent in the composition is typically an LP polypeptide, antagonist or agonist thereof. The label on, or associated with, the container indicates that the composition is used for diagnosing or treating the condition of choice. The article of manufacture may further comprise a second container comprising a pharmaceutically-acceptable buffer, such as phosphate-buffered saline, Ringer's solution and dextrose solution. It may further include other materials desirable from a commercial and user standpoint, including other buffers, diluents, filters, needles, syringes, and package inserts with instructions for use.

Preferred activities and/or therapeutic utilities of the nucleic acids, polypeptides, antibodies, or compositions of the present invention are determined by performing a series of assays. In one embodiment of the invention, Xenopus embryo assays are performed, whereby RNA is injected into the embryo, and the corresponding phenotype is measured. The phenotype is compared to phenotypes of known BMP-binding molecules. Another embodiment involves BMP binding assays, performed to establish that LP polypeptide acts as a BMP antagonist.

In another embodiment of the invention, therapeutic utility of the LP polypeptide is determined by measuring phosphorylation of tyrosine residues on specific cell lines. The early cellular response of cells stimulated with the majority of proteins is protein phosphorylation of the tyrosine residues. This response includes autophosphorylation of corresponding receptors, which thereby leads to the activation of catalytic properties and the initiation of intracellular pathways specific to the cell type. Moreover, signaling downstream of receptors requires phosphorylation of specific kinases inside the cell and other intracellular enzymes of different origin as well as the phosphorylation of multiple adapter/scaffold, structural proteins and transcriptional factors. Therefore, diverse protein-induced cell responses can be visualized by monitoring the state of protein phosphorylation after cell stimulation.

Immunodetection is used to detect the protein phosphorylation of the stimulated cell. Several antibodies that are directed against specific phosphorylated epitopes in signaling molecules are readily available. Two specific antibodies are used: phosphospecific anti-MAPK and anti-AKT antibodies. Additionally, non-specific anti-phosphotyrosine antibodies, which recognize tyrosine-phosphorylated proteins, are used. While anti-phosphotyrosine antibodies allow detection of diverse tyrosine phosphorylated proteins without directly addressing the nature of their identity, the phosphospecific anti-MAPK and anti-AKT antibodies recognize only the corresponding proteins in their phosphorylated form.

Another assay to determine utility of LP polypeptides involves transfection of cell lines with reporter plasmids followed by cell stimulation with an LP polypeptide. Each reporter consists of a defined element, responsive to specific intracellular signaling pathways, upstream of a sequence involving a reporter protein such as luciferase. Upon stimulation of the element, reporter transcription and translation ensues, and the resulting protein levels can be detected using an assay such as a luminescence assay. The cell stimulation period depends on the reporter plasmid used.

For each reporter used, positive controls are designed in the form of agonist cocktails which include approximately maximal stimulatory doses of several ligands known to stimulate the represented signaling pathway. Using this design, the chances of finding a positive stimulus for each cell line is maximized. The caveat, however, is that some cell line/reporter combinations will not be stimulated by the specific agonist cocktail, due to lack of an appropriate ligand in the cocktail. Alternately, the lack of signal induction by an agonist cocktail may be the lack of all signaling components within a particular cell line to activate the transcriptional element. Cell line/reporter combinations with no exogenous stimulus added make up the negative controls.

In another assay, utility of LP polypeptide is determined by proliferation of cells. In this assay, gross changes in the number of cells remaining in a culture are monitored after exposure to an LP polypeptide for three days. The cells are incubated in an appropriate assay medium to produce a sub-optimal growth rate. For example, usually a 1:10 or 1:20 dilution of normal culture medium results in a 40 to 60% reduction in cell number compared to the undiluted culture medium. This broad growth zone is chosen so that if an LP polypeptide is a stimulator of growth, the cells have room to expand, and conversely, if the LP polypeptide is deleterious, a reduction in cell density can be detected. After a period of exposure, the assay media is replaced with media containing a detection agent such as Calcein AM, a membrane-permeant fluorescent dye, allowing quantification of the cell number.

For use in another assay, a FLAG-HIS (FLIS)-tagged version of the LP polypeptide is expressed using mammalian cells such as HEK-293EBNA, COS-7, or HEK293T. The coding region of the cDNA is amplified by PCR of a vector containing a fragment encoding the LP polypeptide. The PCR-generated fragment is cleaved with restriction enzymes and gel-purified. The fragment is then ligated into a mammalian expression vector containing the FLIS epitope tag fused to the C-terminus. Protein expressed by this plasmid construct includes both the FLAG tag (Asp-Tyr-Lys-Asp-Asp-Asp-Asp-Lys) and the 6×His tag at the COOH-terminus of the protein. This tag provides epitopes for commercially available tag-specific antibodies, enabling detection of the protein.

To determine expression of the LP polypeptide in tissues, a protein-binding assay is performed. The fixed tissue sample is exposed to the FLIS-tagged LP polypeptide, followed by exposure to a primary antibody and a secondary antibody containing a fluorescent dye. Tagged LP polypeptide that binds to the antigens in the tissue will fluoresce. Binding of the protein to an antigen in the tissue suggests that the protein is expressed in that tissue. Thus, protein expression can be determined by measuring which tissues fluoresce.

Having generally described the invention, the same will be more readily understood by reference to the following examples, which are provided by way of illustration and are not intended as limiting.

### EXAMPLES

### Example 1

### Expression and Purification of LP226, LP233, or LP236 in E. coli

The bacterial expression vector pQE60 is used for bacterial expression in this example. (QIAGEN, Inc., Chatsworth, Calif.). pQE60 encodes ampicillin antibiotic resistance (“Ampr”) and contains a bacterial origin of replication (“ori”), an IPTG inducible promoter, a ribosome binding site (“RBS”), six codons encoding histidine residues that allow affinity purification using nickel-nitrilo-tri-acetic acid (“Ni-NTA”) affinity resin sold by QIAGEN, Inc., and suitable single restriction enzyme cleavage sites. These elements are arranged such that a DNA fragment encoding a polypeptide can be inserted in such a way as to produce that polypeptide with the six His residues (i.e., a “6×His tag”) covalently linked to the carboxyl terminus of that polypeptide. However, a polypeptide coding sequence can optionally be inserted such that translation of the six His codons is prevented and, therefore, a polypeptide is produced with no 6×His tag.

The nucleic acid sequence encoding the desired portion of LP226, LP233, or LP236 lacking the hydrophobic leader sequence is amplified from a cDNA clone using PCR oligonucleotide primers (based on the sequences presented, e.g., as presented in SEQ ID NO:1), which anneal to the amino terminal encoding DNA sequences of the desired portion of LP226, LP233, or LP236 and to sequences in the construct 3′ to the cDNA coding sequence. Additional nucleotides containing restriction sites to facilitate cloning in the pQE60 vector are added to the 5′ and 3′ sequences, respectively.

For cloning LP226, LP233, or LP236, the 5′ and 3′ primers have nucleotides corresponding or complementary to a portion of the coding sequence of LP226, LP233, or LP236, e.g., as presented in the coding regions of SEQ ID NO:1, 3, and 5, respectively, according to known method steps. One of ordinary skill in the art would appreciate, of course, that the point in a polypeptide coding sequence where the 5′ primer begins can be varied to amplify a desired portion of the complete polypeptide shorter or longer than the mature form.

The amplified nucleic acid fragments and the vector pQE60 are digested with appropriate restriction enzymes and the digested DNAs are then ligated together. Insertion of the LP226, LP233, or LP236 DNA into the restricted pQE60 vector places the LP226, LP233, or LP236 polypeptide coding region including its associated stop codon downstream from the IPTG-inducible promoter and in-frame with an initiating AUG codon. The associated stop codon prevents translation of the six histidine codons downstream of the insertion point.

The ligation mixture is transformed into competent E. coli cells using standard procedures such as those described in Sambrook, et al., 1989; Ausubel, 1987-1998. E. coli strain M15/rep4, containing multiple copies of the plasmid pREP4, which expresses the lac repressor and confers kanamycin resistance (“Kanr”), is used in carrying out the illustrative example described herein. This strain, which is only one of many that are suitable for expressing LP polypeptide, is available commercially from QIAGEN, Inc. Transformants are identified by their ability to grow on LB plates in the presence of ampicillin and kanamycin. Plasmid DNA is isolated from resistant colonies and the identity of the cloned DNA confirmed by restriction analysis, PCR and DNA sequencing.

Clones containing the desired constructs are grown overnight (“O/N”) in liquid culture in LB media supplemented with both ampicillin (100 μg/mL) and kanamycin (25 μg/mL). The O/N culture is used to inoculate a large culture, at a dilution of approximately 1:25 to 1:250. The cells are grown to an optical density at 600 nm (“OD600”) of between 0.4 and 0.6. Isopropyl-b-D-thiogalactopyranoside (“IPTG”) is then added to a final concentration of 1 mM to induce transcription from the lac repressor sensitive promoter, by inactivating the lacI repressor. Cells subsequently are incubated further for 3 to 4 hours. Cells then are harvested by centrifugation.

The cells are then stirred for three to four hours at 4 degrees C. in 6 M guanidine hydrochloride, pH 8. The cell debris is removed by centrifugation, and the supernatant containing LP226, LP233, or LP236 is dialyzed against 50 mM sodium acetate buffer pH 6, supplemented with 200 mM sodium chloride. Alternatively, a polypeptide can be successfully refolded by dialyzing it against 500 mM sodium chloride, 20% glycerol, 25 mM Tris hydrochloride pH 7.4, containing protease inhibitors.

If insoluble protein is generated, the protein is made soluble according to known method steps. After renaturation, the polypeptide is purified by ion exchange, hydrophobic interaction, and size exclusion chromatography. Alternatively, an affinity chromatography step such as an antibody column is used to obtain pure LP226, LP233, or LP236. The purified polypeptide is stored at 4 degrees C. or frozen at negative 40 degrees C. to negative 120 degrees C.

### Example 2

### Cloning and Expression of LP226, LP233, or LP236 in a Baculovirus Expression System

In this example, the plasmid shuttle vector pA2 GP is used to insert the cloned DNA encoding the mature polypeptide into a baculovirus to express LP226, LP233, or LP236, using a baculovirus leader and standard methods as described in Summers, et al., A Manual of Methods for Baculovirus Vectors and Insect Cell Culture Procedures, Texas Agricultural Experimental Station Bulletin No. 1555 (1987). This expression vector contains the strong polyhedrin promoter of the Autographa californica nuclear polyhedrosis virus (AcMNPV) followed by the secretory signal peptide (leader) of the baculovirus gp67 polypeptide and convenient restriction sites such as BamHI, Xba I, and Asp718. The polyadenylation site of the simian virus 40 (“SV40”) is used for efficient polyadenylation. For easy selection of recombinant virus, the plasmid contains the beta-galactosidase gene from E. coli under control of a weak Drosophila promoter in the same orientation, followed by the polyadenylation signal of the polyhedrin gene. The inserted genes are flanked on both sides by viral sequences for cell-mediated homologous recombination with wild-type viral DNA to generate viable virus that expresses the cloned polynucleotide.

Other baculovirus vectors are used in place of the vector above, such as pAc373, pVL941 and pAcIM1, as one skilled in the art would readily appreciate, as long as the construct provides appropriately located signals for transcription, translation, secretion and the like, including a signal peptide and an in-frame AUG as required. Such vectors are described, for instance, in Luckow, et al., Virology 170: 31-9.

The cDNA sequence encoding the mature LP226, LP233, or LP236 polypeptide in a clone, lacking the AUG initiation codon and the naturally associated nucleotide binding site, is amplified using PCR oligonucleotide primers corresponding to the 5′ and 3′ sequences of the gene. Non-limiting examples include 5′ and 3′ primers having nucleotides corresponding or complementary to a portion of the coding sequence of a LP226, LP233, or LP236 polypeptide, e.g., as presented in SEQ ID NO:2, 4, and 6, respectively, according to known method steps.

The amplified fragment is isolated from a 1% agarose gel using a commercially available kit (e.g., “Geneclean,” BIO 101 Inc., La Jolla, Calif.). The fragment then is then digested with the appropriate restriction enzyme and again is purified on a 1% agarose gel. This fragment is designated herein “F1.”

The plasmid is digested with the corresponding restriction enzymes and optionally, can be dephosphorylated using calf intestinal phosphatase, using routine procedures known in the art. The DNA is then isolated from a 1% agarose gel using a commercially available kit (“Geneclean” BIO 101 Inc., La Jolla, Calif.). This vector DNA is designated herein “V1.”

Fragment F1 and the dephosphorylated plasmid V1 are ligated together with T4 DNA ligase. E. coli HB101 or other suitable E. coli hosts such as XL-1 Blue (Stratagene Cloning Systems, La Jolla, Calif.) cells are transformed with the ligation mixture and spread on culture plates. Bacteria are identified that contain the plasmid bearing the human LP226, LP233, or LP236 gene using the PCR method, in which one of the primers that is used to amplify the gene and the second primer is from well within the vector so that only those bacterial colonies containing the LP226, LP233, or LP236 gene fragment will show amplification of the DNA. The sequence of the cloned fragment is confirmed by DNA sequencing. This plasmid is designated herein pBac LP226, LP233, or LP236.

Five μg of the plasmid pBacLP226, LP233, or LP236 is co-transfected with 1.0 μg of a commercially available linearized baculovirus DNA (“BaculoGold® baculovirus DNA”, PharMingen, San Diego, Calif.), using the lipofection method described by Felgner, et al., Proc. Natl. Acad. Sci. USA 84: 7413-7 (1987). 1 μg of BaculoGold® virus DNA and 5 μg of the plasmid pBac LP226, LP233, or LP236 are mixed in a sterile well of a microtiter plate containing 50 μL of serum-free Grace's medium (Life Technologies, Inc., Rockville, Md.). Afterwards, 10 μL Lipofectin plus 90 μL Grace's medium are added, mixed and incubated for 15 minutes at room temperature. Then, the transfection mixture is added drop-wise to Sf9 insect cells (ATCC CRL 1711) seeded in a 35 mm tissue culture plate with 1 mL Grace's medium without serum. The plate is rocked back and forth to mix the newly added solution. The plate is then incubated for five hours at 27 degrees C. After 5 hours the transfection solution is removed from the plate and 1 mL of Grace's insect medium supplemented with 10% fetal calf serum is added. The plate is put back into an incubator and cultivation is continued at 27 degrees C. for four days.

After four days, the supernatant is collected, and a plaque assay is performed. An agarose gel with “Blue Gal” (Life Technologies, Inc., Rockville, Md.) is used to allow easy identification and isolation of gal-expressing clones, which produce blue-stained plaques. (A detailed description of a “plaque assay” of this type can also be found in the user's guide for insect cell culture and baculovirology distributed by Life Technologies, Inc., Rockville, Md., pages 9-10). After appropriate incubation, blue stained plaques are picked with a micropipettor tip (e.g., Eppendorf). The agar containing the recombinant viruses is then resuspended in a microcentrifuge tube containing 200 μL of Grace's medium and the suspension containing the recombinant baculovirus is used to infect Sf9 cells seeded in 35 mm dishes. Four days later, the supernatants of these culture dishes are harvested and then stored at 4 degrees C. The recombinant virus is called V-LP226, V-LP233, or V-LP236.

To verify the expression of LP226, LP233, or LP236, Sf9 cells are grown in Grace's medium supplemented with 10% heat-inactivated FBS. The cells are infected with the recombinant baculovirus V-LP226, LP233, or LP236 at a multiplicity of infection (“MOI”) of about two. Six hours later, the medium is removed and replaced with SF900 II medium minus methionine and cysteine (available, e.g., from Life Technologies, Inc., Rockville, Md.). If radiolabeled polypeptides are desired, 42 hours later, 5 mCi of ³⁵S-methionine and 5 mCi ³⁵S cysteine (available from Amersham, Piscataway, N.J.) are added. The cells are further incubated for sixteen hours and then harvested by centrifugation. The polypeptides in the supernatant as well as the intracellular polypeptides are analyzed by SDS-PAGE, followed by autoradiography (if radiolabeled). Microsequencing of the amino acid sequence of the amino terminus of purified polypeptide can be used to determine the amino terminal sequence of the mature polypeptide and, thus, the cleavage point and length of the secretory signal peptide.

### Example 3

### Cloning and Expression of LP226, LP233, or LP236 in Mammalian Cells

A typical mammalian expression vector contains at least one promoter element, which mediates the initiation of transcription of mRNA, the polypeptide coding sequence, and signals required for the termination of transcription and polyadenylation of the transcript. Additional elements include enhancers, Kozak sequences and intervening sequences flanked by donor and acceptor sites for RNA splicing. Highly efficient transcription can be achieved with the early and late promoters from SV40, the long terminal repeats (LTRS) from Retroviruses, e.g., RSV, HTLVI, HIVI and the early promoter of the cytomegalovirus (CMV). However, cellular elements can also be used (e.g., the human actin promoter). Suitable expression vectors for use in practicing the present invention include, for example, vectors such as pIRES1neo, pRetro-Off, pRetro-On, PLXSN, or PLNCX (Clontech Labs, Palo Alto, Calif.), pcDNA3.1 (+/−), pcDNA/Zeo (+/−) or pcDNA3.1/Hygro (+/−) (Invitrogen), PSVL and PMSG (Pharmacia, Uppsala, Sweden), pRSVcat (ATCC 37152), pSV2dhfr (ATCC 37146) and pBC12MI (ATCC 67109). Other suitable mammalian host cells include human Hela 293, H9, Jurkat cells, mouse NIH3T3, C127 cells, Cos 1, Cos 7 and CV 1, quail QC1-3 cells, mouse L cells and Chinese hamster ovary (CHO) cells.

Alternatively, the gene is expressed in stable cell lines that contain the gene integrated into a chromosome. The co-transfection with a selectable marker such as DHRF (dihydrofolate reductase), GPT neomycin, or hygromycin allows the identification and isolation of the transfected cells.

The transfected gene can also be amplified to express large amounts of the encoded polypeptide. The DHFR marker is useful to develop cell lines that carry several hundred or even several thousand copies of the gene of interest. Another useful selection marker is the enzyme glutamine synthase (GS) (Murphy, et al., Biochem. J. 227: 277-9 (1991); Bebbington, et al., Bio/Technology 10: 169-75 (1992)). Using these markers, the mammalian cells are grown in selective medium and the cells with the highest resistance are selected. These cell lines contain the amplified gene(s) integrated into a chromosome. Chinese hamster ovary (CHO) and NSO cells are often used for the production of polypeptides.

The expression vectors pC1 and pC4 contain the strong promoter (LTR) of the Rous Sarcoma Virus [Cullen, et al., Mol. Cell. Biol. 5: 438-47 (1985)] plus a fragment of the CMV-enhancer [Boshart, et al., Cell 41: 521-30 (1985)]. Multiple cloning sites, e.g., with the restriction enzyme cleavage sites BamHI, XbaI, and Asp718, facilitate the cloning of the gene of interest. The vectors contain in addition the 3′ intron, the polyadenylation and termination signal of the rat preproinsulin gene.

### Example 3(a)

### Cloning and Expression in COS Cells

The expression plasmid, pLP226, LP233, or LP236 HA, is made by cloning a cDNA encoding LP226, LP233, or LP236 into the expression vector pcDNAI/Amp or pcDNAIII (which can be obtained from Invitrogen, Inc.).

The expression vector pcDNAI/amp contains: (1) an E. coli origin of replication effective for propagation in E. coli and other prokaryotic cells; (2) an ampicillin resistance gene for selection of plasmid-containing prokaryotic cells; (3) an SV40 origin of replication for propagation in eukaryotic cells; (4) a CMV promoter, a polylinker, an SV40 intron; (5) several codons encoding a hemagglutinin fragment (i.e., an “HA” tag to facilitate purification) or HIS tag (see, e.g., Ausubel, supra) followed by a termination codon and polyadenylation signal arranged so that a cDNA can be conveniently placed under expression control of the CMV promoter and operably linked to the SV40 intron and the polyadenylation signal by means of restriction sites in the polylinker. The HA tag corresponds to an epitope derived from the influenza hemagglutinin polypeptide described by Wilson, et al., Cell 37: 767-8 (1984). The fusion of the HA tag to the target polypeptide allows easy detection and recovery of the recombinant polypeptide with an antibody that recognizes the HA epitope. pcDNAIII contains, in addition, the selectable neomycin marker.

A DNA fragment encoding the LP226, LP233, or LP236 is cloned into the polylinker region of the vector so that recombinant polypeptide expression is directed by the CMV promoter. The plasmid construction strategy is as follows. The LP226, LP233, or LP236 cDNA of a clone is amplified using primers that contain convenient restriction sites, much as described above for construction of vectors for expression of LP226, LP233, or LP236 in E. coli. Non-limiting examples of suitable primers include those based on the coding sequences presented in SEQ ID NO:1, 3, 5 as they encode LP226, LP233, and LP236, respectively, as described herein.

The PCR amplified DNA fragment and the vector, pcDNAI/Amp, are digested with suitable restriction enzyme(s) and then ligated. The ligation mixture is transformed into E. coli strain SURE (available from Stratagene Cloning Systems, La Jolla, Calif.), and the transformed culture is plated on ampicillin media plates which then are incubated to allow growth of ampicillin resistant colonies. Plasmid DNA is isolated from resistant colonies and examined by restriction analysis or other means for the presence of the LP226, LP233, or LP236-encoding fragment.

For expression of recombinant LP226, LP233, or LP236, COS cells are transfected with an expression vector, as described above, using DEAE-DEXTRAN, as described, for instance, in Sambrook, et al., Molecular Cloning: a Laboratory Manual, Cold Spring Laboratory Press, Cold Spring Harbor, N.Y. (1989). Cells are incubated under conditions for expression of LP226, LP233, or LP236 by the vector.

Expression of the LP226, LP233, or LP236-HA fusion polypeptide is detected by radiolabeling and immunoprecipitation, using methods described in, for example Harlow, et al., Antibodies: A Laboratory Manual, 2nd Ed., Cold Spring Harbor Laboratory Press, Cold Spring Harbor, N.Y. (1988). To this end, two days after transfection, the cells are labeled by incubation in media containing ³⁵S-cysteine for eight hours. The cells and the media are collected, and the cells are washed and lysed with detergent-containing RIPA buffer: 150 mM sodium chloride, 1% NP-40, 0.1% SDS, 0.5% DOC, 50 mM TRIS, pH 7.5, as described by Wilson, et al., cited above. Proteins are precipitated from the cell lysate and from the culture media using an HA-specific monoclonal antibody. The precipitated polypeptides then are analyzed by SDS-PAGE and autoradiography. An expression product of the expected size is seen in the cell lysate, which is not seen in negative controls.

### Example 3(b)

### Cloning and Expression in CHO Cells

The vector pC4 is used for the expression of LP226, LP233, or LP236. Plasmid pC4 is a derivative of the plasmid pSV2-dhfr (ATCC Accession No. 37146). The plasmid contains the mouse DHFR gene under control of the SV40 early promoter. Chinese hamster ovary cells or other cells lacking dihydrofolate activity that are transfected with these plasmids can be selected by growing the cells in a selective medium (alpha minus MEM, Life Technologies) supplemented with methotrexate. The amplification of the DHFR genes in cells resistant to methotrexate (MTX) has been well documented (see, e.g., Alt, et al., J. Biol. Chem. 253: 1357-70 (1978); Hamlin and Ma, Biochem. et Biophys. Acta 1097: 107-43 (1990); and Page and Sydenham, Biotechnology 9: 64-8 (1991)). Cells grown in increasing concentrations of MTX develop resistance to the drug by overproducing the target enzyme, DHFR, as a result of amplification of the DHFR gene. If a second gene is linked to the DHFR gene, it is usually co-amplified and overexpressed. It is known in the art that this approach can be used to develop cell lines carrying more than 1,000 copies of the amplified gene(s). Subsequently, when the methotrexate is withdrawn, cell lines are obtained which contain the amplified gene integrated into one or more chromosome(s) of the host cell.

Plasmid pC4 contains for expressing the gene of interest the strong promoter of the long terminal repeat (LTR) of the Rous Sarcoma Virus [Cullen, et al., Mol. Cell. Biol. 5: 438-47 (1985)] plus a fragment isolated from the enhancer of the immediate early gene of human cytomegalovirus (CMV) [Boshart, et al., Cell 41: 521-30 (1985)]. Downstream of the promoter are BamHI, XbaI, and Asp718 restriction enzyme cleavage sites that allow integration of the genes. Behind these cloning sites, the plasmid contains the 3′ intron and polyadenylation site of the rat preproinsulin gene. Other high efficiency promoters can also be used for the expression, e.g., the human beta-actin promoter, the SV40 early or late promoters or the long terminal repeats from other retroviruses, e.g., HIV and HTLVI. Clontech's Tet-Off and Tet-On gene expression systems and similar systems can be used to express the LP polypeptide in a regulated way in mammalian cells [M. Gossen, and H. Bujard, Proc. Natl. Acad. Sci. USA 89: 5547-51 (1992)]. For the polyadenylation of the mRNA other signals, e.g., from the human growth hormone or globin genes can be used as well. Stable cell lines carrying a gene of interest integrated into the chromosomes can also be selected upon co-transfection with a selectable marker such as gpt, G418 or hygromycin. It is advantageous to use more than one selectable marker in the beginning, e.g., G418 plus methotrexate.

The plasmid pC4 is digested with restriction enzymes and then dephosphorylated using calf intestinal phosphatase by procedures known in the art. The vector is then isolated from a 1% agarose gel.

The DNA sequence encoding the complete LP226, LP233, or LP236 polypeptide is amplified using PCR oligonucleotide primers corresponding to the 5′ and 3′ sequences of the gene. Non-limiting examples include 5′ and 3′ primers having nucleotides corresponding or complementary to a portion of the coding sequences of LP226, LP233, or LP236, e.g., as presented in SEQ ID NO:1, 3, or 5, respectively, according to known method steps.

The amplified fragment is digested with suitable endonucleases and then purified again on a 1% agarose gel. The isolated fragment and the dephosphorylated vector are then ligated with T4 DNA ligase. E. coli HB101 or XL-1 Blue cells are then transformed and bacteria are identified that contain the fragment inserted into plasmid pC4 using, for instance, restriction enzyme analysis.

Chinese hamster ovary (CHO) cells lacking an active DHFR gene are used for transfection. Five μg of the expression plasmid pC4 is cotransfected with 0.5 μg of the plasmid pSV2-neo using lipofectin. The plasmid pSV2-neo contains a dominant selectable marker, the neo gene from Tn5 encoding an enzyme that confers resistance to a group of antibiotics including G418. The cells are seeded in alpha minus MEM supplemented with 1 μg/mL G418. After two days, the cells are trypsinized and seeded in hybridoma cloning plates (Greiner, Germany) in alpha minus MEM supplemented with 10, 25, or 50 ng/mL of methotrexate plus 1 μg/mL G418. After about ten to fourteen days, single clones are trypsinized and then seeded in six-well petri dishes or 10 mL flasks using different concentrations of methotrexate (50 nM, 100 nM, 200 nM, 400 nM, 800 nM). Clones growing at the highest concentrations of methotrexate are then transferred to new six-well plates containing even higher concentrations of methotrexate (1 mM, 2 mM, 5 mM, 10 mM, 20 mM). The same procedure is repeated until clones are obtained which grow at a concentration of 100 to 200 mM. Expression of the desired gene product is analyzed, for instance, by SDS-PAGE and Western blot or by reversed phase HPLC analysis.

### Example 4

### Tissue Distribution of LP226 or LP233 mRNA Expression

Non-radioactive northern blot analysis is performed to examine gene expression in human tissues. First, a control probe, Human G3PDH cDNA Control Probe (Clontech #9805-1), is labeled with digoxigenin using DIG-High Prime Labeling kit (Roche #1585606).

LP226 or LP233 cDNA is cloned into a pPR1 or XenoFLIS vector using PCR conditions known to a person skilled in the art. This may be accomplished using the following PCR mixture: 5 μL PCR buffer and 5 μL PCR DIG mix from PCR DIG Probe Synthesis Kit (Roche #1636090); 0.75 μL Enzyme Mix (Expand High Fidelity); 5 μL of 10 μM pPR1 probe primer (5′-TGCAAAGCTTGGCGCGCC-3′); 5 μL of 10 μM FLAG Probe Primer (5′-CTTGTCGTCGTCATCCTTGTAGTCG-3′); 1 μL plasmid DNA (10 ng); and 28.25 μL sterile water. The cDNA is labeled using this mixture and the following PCR method: one cycle of 95 degrees C. for two minutes; thirty to forty cycles of 95 degrees C. for thirty seconds, 60 degrees C. for thirty seconds, and 70 degrees C. for 1.5 minutes/1 kb of inserted cDNA; one cycle of 72 degrees C. for ten minutes; and a 4 degrees C. soak cycle.

Next, the samples are hybridized to human multiple tissue northern (MTN) blot membranes (Clontech #7780-1) using DIG Easy Hyb (Roche #1603558). The DIG DNA probes prepared above are denatured, diluted with DIG Easy Hyb, and the membranes are incubated overnight with the diluted hybridization/probe mixture.

Detection of the membranes is accomplished using CDP-Star chemiluminescent substrate (Roche #1685627) and X-ray film. The washed membranes are blocked with DIG Blocking Buffer (Wash and Block Buffer Kit, Roche #1585762), and anti-digoxigenin-AP Fab fragments (Roche #1093274) are added. The membranes are equilibrated in detection solution (DIG Wash and Block Buffer Kit, Roche #1585762) then incubated with CDP-Star (Roche #1685627). After lightly blotting, the membranes are placed between two pieces of transparency film and exposed to X-ray film for various exposure times to ensure detection of all possible bands.

Northern blot data indicate that the main tissue of LP226 and LP233 expression is the heart, and thus, these DNA and their respective proteins are useful in the treatment of cardiovascular diseases.

### Example 5

### In Vitro Xenopus Embryo Determinations that LP226, LP233, and LP236 are BMP Antagonists

The human protein chordin acts to inhibit some members of the bone morphogenetic protein family, such as BMP-2 and BMP-4, from binding to their receptors in vivo via cysteine rich domains contained within the chordin protein. Larraín, et al., Development 127(4): 821-30 (2000). LP226, LP233, and LP236 proteins contain one or more of these essential cysteine rich domains.

To determine if LP226, LP233, or LP236 also act as BMP antagonists, animal cap, ventral, and osteogenic differentiation assays are performed. If they act as BMP antagonists, Xenopus embryos injected with RNA from LP226, LP233, or LP236 should result in twinned axes containing dorsal structures. Sasai, et al., Cell 79(5): 779-90 (1994). Alternatively, the embryos may be dorsalized as opposed to having twinned axes. It has been shown that injection of RNA containing just one cysteine rich region of chordin results in dorsalizing activity. Larraín, et al., Development 127(4): 821-30 (2000). One plausible explanation for this effect is that the molecular mass is sufficiently smaller than full-length chordin so that the protein can diffuse farther into the embryo, resulting in a less localized effect. Comparing the proteins of the present invention, LP226, LP233, and LP236 are smaller than wild type chordin. Chordin contains four cysteine-rich repeats. LP226 and LP236 contain three cysteine-rich repeats, and LP233 contains only one. Thus, LP226, LP233, or LP236 may be able to diffuse further into the embryo resulting in dorsalization instead of twinning.

In addition, LP226, LP233, and LP236 do not contain known tolloid cleavage sites that are present in wild type chordin. Tolloid cleaves human chordin at the N-terminus at HRSYS . . . DRGEP and at the C-terminus at DPMQA . . . DGPRG. Scott, et al., Dev. Biol. 213(2): 283-300 (1999). That cleavage renders chordin inactive and unable to bind BMPs. Since LP226, LP233, or LP236 theoretically cannot be cleaved by tolloid (and, therefore, cannot be inactivated), they may be active over a wider range within the embryo. Thus, dorsalization of the Xenopus embryos occurs instead of twinning.

To perform in vitro assays that confirm that LP polypeptide is a BMP antagonist, vectors containing DNA of LP polypeptide must be prepared. LP polynucleotide is cloned into an expression vector (EW1969) containing the T7 RNA polymerase binding site. Plasmid DNA containing the LP cDNA insert is linearized with Not I restriction enzyme (Gibco BRL, Rockville, Md.) and in vitro transcribed using Ambion's mESSAGE mACHINE (Ambion, Austin, Tex.) containing T7 RNA polymerase. RNAs produced are examined on an agarose gel to confirm success of the transcription reaction as well as appropriate size of the RNA product. RNA is translated in vitro using the Biotin in vitro translation kit (Roche, Indianapolis, Ind.) to determine if RNA is of sufficient quality to produce protein of predicted molecular weight. Finally, RNA is diluted to 200 μg/mL with RNase free water and stored at negative 20 degrees C. until ready for microinjection into Xenopus embryos.

Next, Xenopus embryos are produced. Female Xenopus frogs are injected with 300 U/frog of human chorionic gonadotropin (Sigma) to induce egg laying. Eggs are harvested from the females and combined with macerated testis to fertilize the eggs in vitro.

Fertilized eggs are dejellied with 2% cysteine in water (pH 8.0), rinsed, and transferred to 1×MR (0.1 M sodium chloride, 1.8 mM potassium chloride, 2.0 mM calcium chloride, 1.0 mM magnesium chloride, and 50 mM HEPES-NaOH). Embryos are developed at room temperature or 15 degrees C. until signs of the first cleavage furrow appeared. Embryos are transferred to 4% Ficoll (Sigma, St. Louis, Mo.) in 1×MR prior to injection.

### Example 5(a)

### Injection of LP226 RNA into Xenopus Embryos

Once, embryos are produced, LP226 RNA is injected into the Xenopus embryos. Five nanoliters (1 ng) of RNA are injected into both cells of a two-cell stage embryo, two cells (ventral and dorsal) of a four-cell embryo, or one vegetal ventral or dorsal cell of an eight-cell embryo. Embryos are left in the 4% Ficoll/1×MR solution until the embryos were at the blastula stage. They are then switched to 0.1×MR with 4% Ficoll and allowed to develop at 18 degrees C. Embryos are observed for morphological effects during the next two to three days.

Approximately twenty-four hours after injection, morphological effects are noted in injected embryos. Uninjected control embryos develop normally. A few of the LP226 RNA-injected embryos (in each injection paradigm) begin to look as though they are either arrested in gastrulation or delayed in neurulation. Observations at forty-eight hours after injection reveals embryos that are hyperdorsalized. Both dorsally and ventrally injected embryos become hyperdorsalized, in accordance with previous observations for chordin by Sasai, et al. In this experiment, the twinning effect, noted by Sasai when chordin is injected ventrally in embryos, is not seen with LP226. The non-twinning results accord with the hyperdorsalizing effect results of Larraín, achieved when RNA encoding only the cysteine rich domain of chordin is injected. Larraín, et al., Development 127: 821-30 (2000). These results may be explained by the fact that LP226 is approximately 30 kD smaller than wild type chordin, and it doesn't have the tolloid cleavage site. Thus, it is not cleaved by tolloid. Additionally, twinning may not have occurred because the injection volume is five times larger in this experiment than in the Sasai experiment.

In another experiment, less RNA is injected into the ventral cells. The table infra lists injection size and scheme. In this experiment, 200 picogram injections of LP226 RNA induce anteriorization of embryos when injected into both cells of the two-cell stage. One nanogram injections of LP226 RNA induce both dorzalized and twinned embryos when injected into one vegetal cell of the eight-celled embryo. These phenotypes indicate that LP226 is a BMP antagonist.

| | Injection | | % | % | % | % | % | % |
|---|---|---|---|---|---|---|---|---|
| RNA | Scheme | N | surviving | normal | abnormal | dorsalized | twins | anteriorized |
| LP226 | 2 × 2 | 10 | 70 | | | | | 100 |
| | 200 pg | | | | | | | |
| | 1 × 4 v | 10 | 100 | 90 | 10 | | | |
| | 200 pg | | | | | | | |
| | 1 × 4 d | 10 | 80 | 88 | | | | 12 |
| | 200 pg | | | | | | | |
| | 1 × 8 vv | 10 | 80 | 88 | | | | 12 |
| | 200 pg | | | | | | | |
| | 1 × 8 vd | 10 | 100 | 90 | | 10 | | |
| | 200 pg | | | | | | | |
| | 1 × 8 vv | 10 | 70 | 14 | 43 | 29 | 14 | |
| | 1 ng | | | | | | | |
| | 1 × 8 vd | 10 | 60 | 0 | 17 | 67 | 17 | |
| | 1 ng | | | | | | | |
| Uninjected | | 32 | 100 | 97 | 3 | | | |
| (stage 35) | | | | | | | | |
| Water | 2 × 2 | 10 | 70 | 71 | 29 | | | |
| | 5 nL | | | | | | | |
| | 1 × 4 v | 5 | 80 | 100 | | | | |
| | 5 nl | | | | | | | |
| | 1 × 4 d | 5 | 100 | 80 | 20 | | | |
| | 5 nl | | | | | | | |
| | 1 × 8 vv | 10 | 80 | 88 | 12 | | | |
| | 5 nl | | | | | | | |
| | 1 × 8 vd | 10 | 80 | 100 | | | | |
| | 5 nl | | | | | | | |

2 × 2 = two cell embryo, two cells injected
1 × 4 = four cell embryo, one cell injected
1 × 8 = eight cell embryo, one cell injected
v = ventral
vv = vegetal ventral
d = dorsal
vd = vegetal dorsal

### Example 5(b)

### Animal Cap and Ventral Marginal Zone Assays

Animal cap experiments of BMP antagonists are expected to produce animal caps with neural gene expression such as neural cell adhesion molecule (NCAM) but little morphological effects. Ventral marginal zone explants are expected to be morphologically elongated and to express neural genes while endodermal genes are suppressed.

In these experiments, the RNA injected embryos prepared above at stage 8 [Nieuwkoop and Faber, A Normal Table of Xenopis laevis (Daudin) (1967)] are harvested for animal cap studies. Vitelline membranes are removed from the embryos. Next, a sheet of cells from the animal pole (animal cap) is isolated. The animal caps are grown in a 96-well tissue culture dish (Costar) with animal cap buffer [0.2×MR, 1% BSA (Sigma), and 59 μg/mL Gentamicin (Sigma)] overnight at room temperature until control embryos reach approximately stage 17 to 19 (Nieuwkoop and Faber, 1967).

Animal cap assays are also performed with uninjected embryos and the addition of LP protein to the animal cap buffer. Ventral marginal zone explants are cultured similar to animal caps, and the effects of the RNA injection or protein addition to uninjected embryos are monitored.

Results of these assays are monitored by morphology, RT-PCR for tissue specific gene expression, and immunohistochemistry for visualization of tissue type and distribution within the explant. Data exhibit that eighteen hours after injection, no morphological changes in the animal cap are observed. These data are consistent with BMP antagonism.

### Example 5(c)

### Osteogenic Differentiation Assays

The osteogenic differentiation assay is a preliminary assay determining if LP226, LP233, or LP236 is a BMP antagonist in a mammalian system. EMPs differentiate 10T1/2 cells into osteoblasts. This differentiation can be inhibited by addition of chordin to the cell culture medium. Alkaline phosphatase is an enzyme produced by osteoblasts. Therefore, BMP addition to the cells increases the production of alkaline phosphatase within the cultures. BMP antagonists would inhibit the production of alkaline phosphatase within the cultures. LP proteins acting as BMP antagonists inhibit or reduce BMP-stimulated alkaline phosphatase production by the cultured cells.

A cell line established from an early mouse embryo, C3H10T1/2 (subsequently referred to as 10T1/2), differentiates into osteoblastic cells when treated with BMP-2, BMP-4, and BMP-7. Katagiri, et al., Biochem. Biophys. Res. Comm. 172(1): 295-9 (1990); Piccolo, et al., Cell 86(4): 589-98 (1996). This assay is used to test if LP polypeptide can inhibit the effects of BMPs in the 10T1/2 cells. 10T1/2 cells are cultured in Eagle's Basal Medium (Dulbecco's modified minimal essential medium) with 10% fetal calf serum (Gibco). Cells are plated in ninety-six well tissue culture plates (Corning) at 4000 cells per well. After twenty-four hours, cells are switched to fresh medium containing 2.5 nM BMP and/or 10 nM LP polypeptide. Retinoic acid (Sigma) is used as a positive control at 0.1 μM with or without 10 nM LP proteins. After forty-eight hours, cells are lysed and substrate solution (Pierce) is added. The lysed cells are incubated at 37 degrees C. for thirty minutes. The reaction is stopped with 0.1 N sodium hydroxide, and the absorbance at 405 nM is measured.

### Example 6

### TGF-beta Superfamily Binding Assays

A BIAcore 2000 instrument is used to detect real-time binding between immobilized LP226 and various TGF-beta and BMP proteins. LP226 is diluted to a concentration of 50 g/mL in 10 mM sodium acetate buffer at pH 5.0. LP226 is immobilized to a CM5 sensor chip using the amine coupling method. Follistatin, containing BSA, is immobilized as a positive control.

TGF-beta and BMP proteins are diluted in HBS-EP buffer. Samples are injected over LP226 and control surfaces using the kinject method. Samples containing 5 g/mL and 1 μg/mL of protein are injected at 30 μL/min for three minutes with a ninety second dissociation time. LP226 and control surfaces are then regenerated with glycine-hydrochloride at pH 3.0.

Strong binding is observed between LP226 and BMP-4, TGF-beta 1, TGF-beta 1.2, or TGF-beta 2. TGF-beta 1, TGF-beta 1.2, or TGF-beta 2 bind to LP226 with a greater affinity (K_{D} of approximately 10⁻⁸) than BMP-4 (K_{D} of approximately 10⁻⁶). Weak or no binding is observed between LP226 and BMP-2 or TGF-beta 3. No binding was observed between LP226 and BMP-5, BMP-6, or activin. No binding is observed with a negative control, fas-ligand, also injected over the LP226 surface. These results indicate that LP226 is a TGF-beta binding protein as well as a BMP binding protein.

### Example 7

### Protein Phosphorylation on Tyrosine Residues

Protein-induced cell responses are determined by monitoring tyrosine phosphorylation upon stimulation of cells by addition of LP226, LP233, or LP236 proteins. This is accomplished in two steps: cell manipulation and immunodetection.

Protein phosphorylation is measured using the following cell lines:

U373MG, MCF-7 (ATCC HTB-22)

balb/c 3T3 (ATCC CCL-163)

HDF (dermal fibroblasts) (Clonetics # CC2511T150)

M07E (leukemia cell line)

On day one, the cells are plated into poly-D-lysine-coated, 96 well plates containing cell propagation medium [DMEM:F12 (3:1), 20 mM HEPES at pH 7.5, 5% FBS, and 50 μg/mL Gentamicin]. The cells are seeded at a concentration of 20,000 cells per well in 100 μL medium. On day two, the propagation medium in each well is replaced with 100 μL starvation medium containing DMEM:F12 (3:1), 20 mM HEPES at pH 7.5, 0.5% FBS, and 50 μg/mL Gentamicin. The cells are incubated overnight.

On day three, pervanadate solution is made ten minutes before cell lysis; pervanadate is prepared by mixing 100 μL of sodium orthovanadate (100 mM) and 3.4 μL of hydrogen peroxide (producing 100× stock pervanadate solution). The lysis buffer is then prepared: 50 mM HEPES at pH 7.5, 150 mM sodium chloride, 10% glycerol, 1% TRITON-X100, 1 mM EDTA, 1 mM pervanadate, and BM protease inhibitors. The cells are stimulated by adding 10 μL of LP226, LP233, or LP236 protein solution to the cells, and incubating for ten minutes. Next, the medium is aspirated, and 75 μL lysis buffer are added to each well. The cells are lysed at 4 degrees C. for fifteen minutes, then 25 μL of 4× loading buffer are added to the cell lysates. The resultant solution is mixed then heated to 95 degrees C.

Detection of tyrosine phosphorylation is accomplished by Western immunoblotting. Twenty microliters of each cell sample are loaded onto SDS-PAGE eight to sixteen percent amino acid-ready gels from Bio-Rad, and the gels are run. The proteins are electrotransferred in transfer buffer (25 mM Tris base at pH 8.3, 0.2 M glycine, 20% methanol) from the gel to a nitrocellulose membrane using 250 mA per gel over a one hour period. The membrane is incubated for one hour at ambient conditions in blocking buffer consisting of TBST (20 mM Tris hydrochloride at pH 7.5, 150 mM sodium chloride, 0.1% TWEEN®-20) with 1% BSA.

Next, the antibodies are added to the membrane. The membrane is incubated overnight at 4 degrees C. with gentle rocking in primary antibody solution consisting of the antibody, TBST, and 1% BSA. The next day, the membrane is washed three times, five minutes per wash, with TBST. The membrane is then incubated in the secondary antibody solution consisting of the antibody, TBST, and 1% BSA for one hour at ambient conditions with gentle rocking. After the incubation, the membrane is washed four times with TBST, ten minutes per wash.

Detection is accomplished by incubating the membrane with 10 to 30 mL of SuperSignal solution for one minute at ambient conditions. After one minute, excess developing solution is removed, and the membrane is wrapped in plastic wrap. The membrane is exposed to X-ray film for twenty second, one minute, and two minute exposures (or longer if needed). The number and intensity of immunostained protein bands are compared to bands for the negative control-stimulated cells (basal level of phosphorylation) by visual comparison.

LP233 stimulates tyrosine phosphorylation in the following cell lines: U373 (nerve cell line); balb/c (fibroblast cell line); HDF (human dermal fibroblast cell line); and M07E (bone marrow cell line).

### Example 8

### Cell Stimulation with Detection Utilizing Reporters

Protein-induced cell responses are measured using reporters. The following cell line/reporter combinations are used:

| cell | reporter element |
|---|---|
| SK-N-MC (ATCC HTB-10) | AP-1 |
| LG (ATCC CRL-1458) | AP-1 |
| GLUTag (SV40 Tag transformed | AP-1 |
| enteroendocrine cell line) | |
| Mv1Lu (mink lung epithelial cells) | p3TP-Lux |

For each reporter used, positive controls are designed in the form of agonist cocktails. These cocktails included approximate maximal stimulatory doses of several ligands known to stimulate the regulated signal pathway. The following agonist cocktails are used as positive controls:

| element | pathway | agonist cocktail |
|---|---|---|
| p3TP-Lux | SMAD | TGF-b1, b3, activin A |
| AP-1 | MAP-kinase | thrombin, PDGF, TNF-alpha, EGF |

Cell lines and reporters with no exogenous stimulus added are used as negative controls.

### Example 8(a)

### Cell Stimulation with AP-1 Reporter

At time zero, the cells are transiently transfected with the reporter plasmids in tissue culture flasks using a standard optimized protocol for all cell lines. After twenty-four hours, the cells are trypsinized and seeded into 96-well poly-D-lysine coated assay plates at a rate of 20,000 cells per well in growth medium. After four to five hours, the medium is replaced with serum-free growth medium. At that time, stimulants for those reporters which required a twenty-four hour stimulation period are added. After forty-eight hours, stimulants for the reporters which required a fuve-hour stimulation period are added. Five hours later, all conditions are lysed using a lysis/luciferin cocktail, and the fluorescence of the samples is determined using a Micro Beta reader.

Each assay plate is plated to contain four positive control wells, sixteen negative control wells, and sixty-four test sample wells (two replicates of thirty-two test samples). The threshold value for a positive “hit” is a fluorescence signal equal to the mean plus two standard deviations of the negative control wells. Any test sample that, in both replicates, generates a signal above that threshold is defined as a “hit.”

LP226 exhibits hits in the SK-N-MC cell line (neuroblastoma); the L6 cell line (skeletal muscle); and the GLUTag cell line (enteroendocrine); all utilizing the AP1 reporter.

### Example 8(b)

### Dose Response Curve for Mv1Lu Cell Stimulation with p3TP-Lux Reporter

Mv1Lu cells (mink lung epithelial) with a p3TP-Lux reporter are used to confirm the TGF-beta antagonist activity of LP226. Extracellular TGF-beta activates the SMAD signal transduction pathway by binding to a receptor on the cell surface of Mv1LU cells. Addition of a TGF-beta antagonist binds the TGF-beta, inhibiting signal transduction.

In this experiment, varying concentrations of LP226 plus 10 pM TGF-beta are added to cells. As the concentration of LP226 increases, the signal transduction decreases. This indicates that LP226 is binding the extracellular TGF-beta so that it cannot bind with the cellular receptor to induce signal transduction. Thus, LP226 does act as a TGF-beta antagonist.

| LP226 dose | | | | | | | |
|---|---|---|---|---|---|---|---|
| (μg) | well 1* | well 2 | well 3 | well 4 | well 5 | well 6 | well 7 |
| 0.43 | 407945 | 458555 | 492657 | 543858 | 506206 | 428542 | 429045 |
| 0.143 | 457359 | 486216 | 542700 | 558722 | 492441 | 426377 | 407925 |
| 0.0478 | 396461 | 420099 | 581011 | 579469 | 508581 | 443027 | 427839 |
| 0.0159 | 387434 | 414456 | 550824 | 556584 | 496871 | 447044 | 392724 |
| 0.00531 | 385304 | 394466 | 497377 | 514956 | 487083 | 453830 | 376538 |
| 0.00177 | 337902 | 383384 | 448270 | 469618 | 442344 | 449536 | 339264 |

*Data for wells in CCPS units, corresponding to fluorescence response on the MicroBeta reader.

### Example 9

### Cell Proliferation and Cytotoxicity Determination Utilizing Fluorescence Detection

This assay is designed to monitor gross changes in the number of cells remaining in culture after exposure to LP proteins for a period of three days. The following cells are used in this assay:

Saos (osteosarcoma cell line)

LNCAP (ATCC CRL-1740)

GLUTag (SV40 Tag transformed enteroendocrine cell line)

HUVEC (Clonetics # CC2517T150)

T1165 (plasmacytoma cell line)

Prior to assay, cells are incubated in an appropriate assay medium to produce a sub-optimal growth rate, e.g., a 1:10 or 1:20 dilution of normal culture medium. Cells are grown in T-150 flasks, then harvested by trypsin digestion and replated at 40 to 50% confluence into poly-D-lysine-treated 96-well plates. Cells are only plated into the inner thirty-two wells to prevent edge artifacts due to medium evaporation; the outer wells are filled with buffer alone. Following incubation overnight to stabilize cell recovery, LP226, LP233, or LP236 proteins are added to the appropriate wells. Each protein is assayed in triplicate at two different concentrations, 1× and 0.1× dilution in assay medium. Two controls are also included on each assay plate: assay medium and normal growth medium.

After approximately 72 hours of exposure, the plates are processed to determine the number of viable cells. Plates are spun to increase the attachment of cells to the plate. The medium is then discarded, and 50 μL of detection buffer is added to each well. The detection buffer consisted on MEM medium containing no phenol red (Gibco) with calcein AM (Molecular Probes) and PLURONIC F-127 (Molecular Probes), each at a 1:2000 dilution. After incubating the plates in the dark at room temperature for thirty minutes, the fluorescence intensity of each well is measured using a Cytofluor 4000-plate reader (PerSeptive Biosystems). For a given cell type, the larger the fluorescence intensity, the greater the number of cells in the well. To determine the effects on cell growth from each plate, the intensity of each well containing cells stimulated with LP226, LP233, or LP236 protein is subtracted from the intensity of the wells containing assay medium only (controls). Thus, a positive number indicated stimulation of cell growth; a negative number indicated a reduction in growth. Additionally, confidence limits at 95 and 90% are calculated from the mean results. Results lying outside the 95% confidence limit are scored as “definite hits.” Results lying between the 95 and 90% confidence limits are scored as “maybes.” The distinction between definite hits and maybes varied due to intraplate variability; thus, subjective scoring is used as a final determination for “hits.”

LP226 increases proliferation in Saos, an osteosarcoma cell line; LNCAP, a prostate carcinoma cell line; GLUTag, an enteroendocrine cell line; and HUVEC, a human umbilical vein endothelial cell line. LP233 increases proliferation in T1165, a plasmocytoma cell line, as well as LNCAP, a prostate carcinoma cell line.

### Example 10

### Determination of Protein Binding in Human Tissue

Binding of LP proteins to human tissues is determined by protein staining with fluorescent dye. The following human tissues are used in this assay:

| Tissue | Specific location | Protein hit |
|---|---|---|
| kidney | glomeruli | LP236 |
| | interstitium | LP236 |
| liver | hepatocytes | LP226, LP236 |
| pancreas | Islet cells | LP226, LP236 |
| gut | Brunner's Glands | LP226 |
| | muscularis (small intestine | LP226 |
| | and colon) | |
| ampulla | epithelial cells | LP226, LP236 |
| (oviduct) | muscularis | LP226 |
| ovary | stroma | LP236 |
| uterus | myometrium | LP226 |
| skin | epidermis | LP226, LP236 |
| breast | ductile epithelial cells | LP226 |

All tissues are fixed with 3% paraformaldehyde and embedded in paraffin.

Tissues are prepared for analysis by removing the paraffin with xylene then gradually rehydrating the tissue with graded solutions of ethanol and water. Antigen retrieval is performed to unmask antigenic sites so that antibodies can recognize the antigen. This is accomplished by soaking the tissue in citrate buffer (Dako, Carpinteria, CA) for twenty minutes at 80 to 90 degrees C. followed ten minutes at ambient temperature. The tissue is then washed in tris-buffered saline (TBS) containing 0.05% TWEEN®-20 and 0.01% thimerosol. To minimize non-specific background staining, the tissue is soaked in non-serum protein block (Dako) for forty-five minutes, after which the protein block is removed by blowing air over the tissue.

The tissue is exposed for two hours to the FLAG-HIS tagged LP226, LP233, or LP236 protein at 10 μg/mL. Following exposure, the tissue is washed twice with tris-buffered saline (TBS) containing 0.05% TWEEN®-20 and 0.01% thimerosol. The tissue sample is then incubated for one hour with mouse anti-FLAG antibody at 10 μg/mL. Subsequently, the tissue is washed twice with tris-buffered saline (TBS) containing 0.05% TWEEN®-20 and 0.01% thimerosol. Next, the tissue is exposed to rabbit anti-mouse Ig with Alexa 568, a fluorescent dye, at 10 μg/mL for one hour, followed again by two washes with tris-buffered saline (TBS) containing 0.05% TWEEN®-20 and 0.01% thimerosol. Finally, the tissue is coverslipped with fluorescence mounting media, and the fluorescence is measured. A positive fluorescence reading indicates that the protein binds with antigens on the tissue, suggesting that the protein is expressed in that tissue.

What is claimed is:

1. Isolated nucleic acid comprising DNA having at least an 90% sequence identity to nucleic acid selected from the group consisting of:
(a) a DNA molecule encoding an LP226 polypeptide comprising the sequence of amino acid residues 1 or about 27 through 430, inclusive, of SEQ ID NO:2;
(b) a DNA molecule encoding an LP233 polypeptide comprising the sequence of amino acid residues 1 or about 27 through 162, inclusive, of SEQ ID NO:4;
(c) a DNA molecule encoding an LP236 polypeptide comprising the sequence of amino acid residues 1 or about 27 through 451, inclusive, of SEQ ID NO:6; and,
(d) the complement of the DNA molecule of (a), (b), or (c).

2. The nucleic acid of claim 1, wherein said DNA comprises the sequence of corresponding nucleotides selected from the group consisting of:
(a) nucleotide positions 28 or about 109 through 1315, inclusive, of SEQ ID NO:1;
(b) nucleotide positions 234 or about 315 through 720, inclusive, of SEQ ID NO:3; and,
(c) nucleotide positions 307 or about 387 through 1660, inclusive, of SEQ ID NO:5.

3. The nucleic acid of claim 1, wherein said DNA comprises the nucleotide sequences selected from the group consisting of SEQ ID NO:1, SEQ ID NO:3, and SEQ ID NO:5.

4. The isolated nucleic acid molecule of claim 1 comprising a nucleotide sequence that encodes the sequence of amino acid residues selected from the group consisting of:
(a) from 1 or about 27 to 430 of SEQ ID NO:2;
(b) from 1 or about 27 to 162 of SEQ ID NO:4; and,
(c) from 1 or about 27 to 451 of SEQ ID NO:6.

5. An isolated nucleic acid molecule encoding an LP polypeptide comprising DNA that hybridizes to the complement of the nucleic acid sequence that encodes amino acids selected from the group consisting of:
(a) from 1 or about 27 to 430 of SEQ ID NO:2;
(b) from 1 or about 27 to 162 of SEQ ID NO:4; and,
(c) from 1 or about 27 to 307 of SEQ ID NO:6.

6. The isolated nucleic acid molecule of claim 5, wherein the isolated nucleic acid sequence is selected from the group consisting of:
(a) nucleotides 28 or about 109 to 1315, inclusive, of SEQ ID NO:1;
(b) nucleotides 234 or about 315 to 720, inclusive, of SEQ ID NO:3; and
(c) nucleotides 307 or about 387 to 1660, inclusive, of SEQ ID NO:5.

7. The isolated nucleic acid molecule of claim 5, wherein hybridization occurs under stringent hybridization and wash conditions.

8. An isolated nucleic acid molecule comprising DNA encoding a polypeptide scoring at least 90% positives when compared to the sequence of amino acid residues selected from the group consisting of:
(a) amino acid residues 1 or 27 through 430 inclusive, of SEQ ID NO:2;
(b) amino acid residues 1 or 27 through 162 inclusive, of SEQ ID NO:4; and,
(c) amino acid residues 1 or 27 through 451 inclusive, of SEQ ID NO:6.

9. A vector comprising the nucleic acid molecule of any of claims 1 to 8.

10. The vector of claim 9, wherein said nucleic acid molecule is operably linked to control sequences recognized by a host cell transformed with the vector.

11. A host cell comprising the vector of claim 10.

12. The host cell of claim 11, wherein said cell is a mammalian cell.

13. The host cell of claim 12, wherein said cell is a CHO cell.

14. The host cell of claim 11, wherein said cell is an E. coli cell.

15. A process for producing an LP polypeptide comprising culturing the host cell of claim 11 under conditions suitable for expression of said LP polypeptide and recovering said LP polypeptide from the cell culture.

16. An isolated polypeptide comprising an amino acid sequence comprising at least about 90% sequence identity to the sequence comprising amino acid residues selected from the group consisting of:
(a) amino acid residues 1 or about 27 through 430, inclusive, of SEQ ID NO:2;
(b) amino acid residues 1 or about 27 through 162, inclusive, of SEQ ID NO:4; and,
(c) amino acid residues 1 or about 27 through 451, inclusive, of SEQ ID NO:6.

17. The isolated LP polypeptide of claim 16 comprising amino acid residues selected from the group consisting of:
(a) amino acid residues 1 or about 27 through 430, inclusive, of SEQ ID NO:2;
(b) amino acid residues 1 or about 27 through 162, inclusive, of SEQ ID NO:4; and,
(c) amino acid residues 1 or about 27 through 451, inclusive, of SEQ ID NO:6.

18. An isolated LP polypeptide scoring at least 90% positives when compared to the sequence of amino acids selected from the group consisting of:
(a) amino acid residues 1 or 27 through 430, inclusive, of SEQ ID NO:2;
(b) amino acid residues 1 or 27 through 162, inclusive, of SEQ ID NO:4; and,
(c) amino acid residues 1 or 27 through 451, inclusive, of SEQ ID NO:6.

19. An isolated polypeptide produced by the method of claim 15.

20. A chimeric molecule comprising an LP polypeptide fused to a heterologous amino acid sequence.

21. The chimeric molecule of claim 20, wherein said heterologous amino acid sequence is an Fc region of an immunoglobulin.

22. An antibody which specifically binds to an LP polypeptide.

23. The antibody of claim 22, wherein said antibody is a monoclonal antibody.

24. The antibody of claim 22, wherein said antibody is selected from the group consisting of a humanized antibody and a human antibody.

25. An agonist to an LP polypeptide.

26. An antagonist to an LP polypeptide.

27. A composition comprising a therapeutically effective amount of an active agent selected from the group consisting of: (a) an LP polypeptide, (b) an agonist to an LP polypeptide, (c) an antagonist to an LP polypeptide, and (d) an anti-LP antibody; in combination with a pharmaceutically acceptable carrier.

28. A method of treating a neurological disorder comprising administering a therapeutically effective amount of an LP agonist or antagonist to a mammal suffering from said disorder.

29. A method of treating a disorder requiring wound or tissue repair comprising administering a therapeutically effective amount of an LP agonist or antagonist to a mammal suffering from said disorder.

30. The method of claim 29 wherein the disorder being treated is selected from the group consisting of a bone fracture, spinal cord injury, pressure ulcer, diabetic ulcer, venous stasis ulcer, and a burn.

31. A method of treating a cardiovascular disorder comprising administering a therapeutically effective amount of an LP agonist or antagonist to a mammal suffering from said disorder.

32. A method of treating a proliferative disorder comprising administering a therapeutically effective amount of an LP agonist or antagonist to a mammal suffering from said disorder.

33. The method of claim 32 wherein the proliferative disorder is selected from the group consisting of osteosarcoma, breast cancer, and prostate cancer.

34. A method of treating a disorder associated with loss of bone mineral density comprising administering a therapeutically effective amount of an LP agonist or antagonist to a mammal suffering from said disorder.

35. A method of treating an osteopenia-related disorder comprising administering a therapeutically effective amount of an LP agonist or antagonist to a mammal suffering from said disorder.

36. A method of treating a fibrotic disorder comprising administering a therapeutically effective amount of an LP agonist or antagonist to a mammal suffering from said disorder.

37. Use of LP polypeptide in the manufacture of a medicament.

38. An article of manufacture comprising a container, label and therapeutically effective amount of an LP polypeptide, LP agonist, or LP antagonist in combination with a pharmaceutically effective carrier.


SEQUENCE LISTING (6 sequences)

SEQ ID NO: 1 - DNA, 1405 bp, Homo sapiens
  CDS (28)..(1314)
  sig_peptide (28)..(384)
gaccagcggcctgaccctggggaaaggatggttcccgaggtgagggtcctctcctccttgctgggactcgcgctgctctggttccccctggactcccacgctcgagcccgcccagacatgttctgccttttccatgggaagagatactcccccggcgagagctggcacccctacttggagccacaaggcctgatgtactgcctgcgctgtacctgctcagagggcgcccatgtgagttgttaccgcctccactgtccgcctgtccactgcccccagcctgtgacggagccacagcaatgctgtcccaagtgtgtggaacctcacactccctctggactccgggccccaccaaagtcctgccagcacaacgggaccatgtaccaacacggagagatcttcagtgcccatgagctgttcccctcccgcctgcccaaccagtgtgtcctctgcagctgcacagagggccagatctactgcggcctcacaacctgccccgaaccaggctgcccagcacccctcccgctgccagactcctgctgccaagcctgcaaagatgaggcaagtgagcaatcggatgaagaggacagtgtgcagtcgctccatggggtgagacatcctcaggatccatgttccagtgatgctgggagaaagagaggcccgggcaccccagcccccactggcctcagcgcccctctgagcttcatccctcgccacttcagacccaagggagcaggcagcacaactgtcaagatcgtcctgaaggagaaacataagaaagcctgtgtgcatggcgggaagacgtactcccacggggaggtgtggcacccggccttccgtgccttcggccccttgccctgcatcctatgcacctgtgaggatggccgccaggactgccagcgtgtgacctgtcccaccgagtacccctgccgtcaccccgagaaagtggctgggaagtgctgcaagatttgcccagaggacaaagcagaccctggccacagtgagatcagttctaccaggtgtcccaaggcaccgggccgggtcctcgtccacacatcggtatccccaagcccagacaacctgcgtcgctttgccctggaacacgaggcctcggacctggtggagatctacctctggaagctggtaaaaggaatcttccacttgactcagatcaagaaagtcaggaagcaagacttccagaaagaggcacagcacttccgactgctcgctggcccccacgaaggtcactggaacgtcttcctagcccagaccctggagctgaaggtcacggccagtccagacaaagtgaccaagacataacaaagacctaacagttgcagatatgagctgtataattgttgttattatatattaataaataagaagttgcattaccctcaaaaaaaaa

SEQ ID NO: 2 - protein, 429 aa, Homo sapiens
MVPEVRVLSSLLGLALLWFPLDSHARARPDMFCLFHGKRYSPGESWHPYLEPQGLMYCLRCTCSEGAHVSCYRLHCPPVHCPQPVTEPQQCCPKCVEPHTPSGLRAPPKSCQHNGTMYQHGEIFSAHELFPSRLPNQCVLCSCTEGQIYCGLTTCPEPGCPAPLPLPDSCCQACKDEASEQSDEEDSVQSLHGVRHPQDPCSSDAGRKRGPGTPAPTGLSAPLSFIPRHFRPKGAGSTTVKIVLKEKHKKACVHGGKTYSHGEVWHPAFRAFGPLPCILCTCEDGRQDCQRVTCPTEYPCRHPEKVAGKCCKICPEDKADPGHSEISSTRCPKAPGRVLVHTSVSPSPDNLRRFALEHEASDLVEIYLWKLVKGIFHLTQIKKVRKQDFQKEAQHFRLLAGPHEGHWNVFLAQTLELKVTASPDKVTKT

SEQ ID NO: 3 - DNA, 1517 bp, Homo sapiens
  CDS (235)..(720)
  sig_peptide (235)..(315)
ctctccctcctttcccgcgttctctttccacctttctcttcttcccaccttagacctcccttcctgccctcctttcctgcccactgctgcttcctggcccttctccgaccccgctctagcagcagacctcctggggtctgtgggttgatctgtggcccctgtgcctccgtgtccttttcgtctcccttcctcccgactccgctcccggaccagcggcctgaccctggggaaaggatggttcccgaggtgagggtcctctcctccttgctgggactcgcgctgctctggttccccctggactcccacgctcgagcccgcccagacatgttctgccttttccatgggaagagatactcccccggcgagagctggcacccctacttggagccacaaggcctgatgtactgcctgcgctgtacctgctcagagggcgcccatgtgagttgttaccgcctccactgtccgcctgtccactgcccccagcctgtgacggagccacagcaatgctgtcccaagtgtgtggaacctcacactccctctggactccgggccccaccaaagtcctgccagcacaacgggaccatgtaccaacacggagagatcttcagtgcccatgagctgttcccctcccgcctgcccaaccagtgtgtcctctgcagctgcacaatgaggcaagtgagcaatcggatgaagaggacagtgtgcagtcgctccatggggtgagacatcctcaggatccatgttccagtgatgctgggagaaagagaggcccgggcaccccagcccccactggcctcagcgcccctctgagcttcatccctcgccacttcagacccaagggagcaggcagcacaactgtcaagatcgtcctgaaggagaaacataagaaagcctgtgtgcatggcgggaagacgtactcccacggggaggtgtggcacccggccttccgtgccttcggccccttgccctgcatcctatgcacctgtgaggatggccgccaggactgccagcgtgtgacctgtcccaccgagtacccctgccgtcaccccgagaaagtggctgggaagtgctgcaagatttgcccagaggacaaagcagaccctggccacagtgagatcagttctaccaggtgtcccaaggcaccgggccgggtcctcgtccacacatcggtatccccaagcccagacaacctgcgtcgctttgccctggaacacgaggcctcggacttggtggagatctacctctggaagctggtaaaaggaatcttccacttgactcagatcaagaaagtcaggaagcaagacttccagaaagaggcacagcacttccgactgctcgctggcccccacgaaggtcactggaacgtcttcctagcccagaccctggagctgaaggtcacggccagtccagacaaagtgaccaagacataacaaagacctaacagttgcagatatgagctgtataattgttgttattatatattaataaataagaagttgcattaccctcaaaaaaaa

SEQ ID NO: 4 - protein, 162 aa, Homo sapiens
MVPEVRVLSSLLGLALLWFPLDSHARARPDMFCLFHGKRYSPGESWHPYLEPQGLMYCLRCTCSEGAHVSCYRLHCPPVHCPQPVTEPQQCCPKCVEPHTPSGLRAPPKSCQHNGTMYQHGEIFSAHELFPSRLPNQCVLCSCTMRQVSNRMKRTVCSRSMG

SEQ ID NO: 5 - DNA, 1738 bp, Homo sapiens
  CDS (307)..(1659)
  sig_peptide (307)..(387)
cgggtcgacccacgcgtccgcccacgcgtccgcgcctctcccttctgctggaccttccttcgtctctccatctctccctcctttccccgcgttctctttccacctttctcttcttcccaccttagacctcccttcctgccctcctttcctgcccaccgctgcttcctggcccttctccgaccccgctctagcagcagacctcctggggtctgtgggttgatctgtggcccctgtgcctccgtgtccttttcgtctcccttcctcccgactccgctcccggaccagcggcctgaccctggggaaaggatggttcccgaggtgagggtcctctcctccttgctgggactcgcgctgctctggttccccctggactcccacgctcgagcccgcccagacatgttctgccttttccatgggaagagatactcccccggcgagagctggcacccctacttggagccacaaggcctgatgtactgcctgcgctgtacctgctcagagggcgcccatgtgagttgttaccgcctccactgtccgcctgtccactgcccccagcctgtgacggagccacagcaatgctgtcccaagtgtgtggaacctcacactccctctggactccgggccccaccaaagtcctgccagcacaacgggaccatgtaccaacacggagagatcttcagtgcccatgagctgttcccctcccgcctgcccaaccagtgtgtcctctgcagctgcacagagggccagatctactgcggcctcacaacctgccccgaaccaggctgcccagcacccctcccactgccagactcctgctgccaagcctgcaaagatgaggcaagtgagcaatcggatgaagaggacagtgtgcagtcgctccatggggtgagacatcctcaggatccatgttccagtgatgctgggagaaagagaggcccgggcaccccagcccccactggcctcagcgcccctctgagcttcatccctcgccacttcagacccaagggagcaggcagcacaactgtcaagatcgtcctgaaggagaaacataagaaagcctgtgtgcatggcgggaagacgtactcccacggggaggtgtggcacccggccttccgtgccttcggccccttgccctgcatcctatgcacctgtgaggatggccgccaggactgccagcgtgtgacctgtcccaccgagtacccctgccgtcaccccgagaaagtggctgggaagtgctgcaagatttgcccagaggacaaagcagaccctggccacagtgagatcagttctaccaggtgtcccaaggcaccgggccgggtcctcgtccacacatcggtatccccaagcccagacaacctgcgtcgctttgccctggaacacgaggcctcggacttggtggagatctacctctggaagctggtaaaagatgaggaaactgaggctcagagaggtgaagtacctggcccaaggccacacagccagaatcttccacttgactcagatcaagaaagtcaggaagcaagacttccagaaagaggcacagcacttccgactgctcgctggcccccacgaaggtcactggaacgtcttcctagcccagaccctggagctgaaggtcacggccagtccagacaaagtgaccaagacataacaaagacctaacagttgcagatatgagctgtataattgttgttattatatattaataaataagaagttgcattaccctcaaaaaaaa

SEQ ID NO: 6 - protein, 451 aa, Homo sapiens
MVPEVRVLSSLLGLALLWFPLDSHARARPDMFCLFHGKRYSPGESWHPYLEPQGLMYCLRCTCSEGAHVSCYRLHCPPVHCPQPVTEPQQCCPKCVEPHTPSGLRAPPKSCQHNGTMYQHGEIFSAHELFPSRLPNQCVLCSCTEGQIYCGLTTCPEPGCPAPLPLPDSCCQACKDEASEQSDEEDSVQSLHGVRHPQDPCSSDAGRKRGPGTPAPTGLSAPLSFIPRHFRPKGAGSTTVKIVLKEKHKKACVHGGKTYSHGEVWHPAFRAFGPLPCILCTCEDGRQDCQRVTCPTEYPCRHPEKVAGKCCKICPEDKADPGHSEISSTRCPKAPGRVLVHTSVSPSPDNLRRFALEHEASDLVEIYLWKLVKDEETEAQRGEVPGPRPHSQNLPLDSDQESQEARLPERGTALPTARWPPRRSLERLPSPDPGAEGHGQSRQSDQDITKT